(19) United States
(12) Patent Application Publication
(10) Pub. No.: US 2023/0278966 A1
(43) Pub. Date: Sep. 7, 2023

(54) TRIAZOLONE COMPOUNDS FOR CONTROLLING INVERTEBRATE PESTS

(71) Applicant: FMC Corporation, Philadelphia, PA (US)

(72) Inventors: Moumita KAR, Hyderabad (IN); Jeffrey Keith LONG, Wilmington, DE (US)

(21) Appl. No.: 18/018,074

(22) PCT Filed: Jul. 28, 2021
(86) PCT No.: PCT/US2021/043396

§ 371 Date: Jan. 26, 2023

Related U.S. Application Data
(60) Provisional application No. 63/058,096, filed on Jul. 29, 2020.

(51) Int. Cl. C07D 249/12; C07D 403/14; C07D 401/14; A01N 43/653

(52) U.S. Cl. CPC C07D 249/12; C07D 403/14; C07D 401/14; A01N 43/653

(57) ABSTRACT

Disclosed are compounds of Formula 1 or Formula 1′, including all geometric and stereoisomers, N-oxides, and salts thereof,

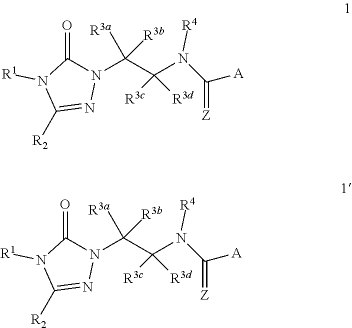

wherein R¹, R², R^{3a}, R^{3b}, R^{3c}, R^{3d}, R⁴, Z and A, respectively, are as defined in the disclosure.
   Also disclosed are compositions containing the compounds of Formula 1 or Formula 1′ and methods for controlling an invertebrate pest comprising contacting the invertebrate pest or its environment with a biologically effective amount of a compound or a composition of the disclosure.

### CROSS-REFERENCE TO RELATED APPLICATIONS

This application claims the benefit of U.S. Provisional Application No. 63/058,096 filed Jul. 29, 2020.

### FIELD

This disclosure relates to certain triazolone compounds, their N-oxides, salts and compositions suitable for agronomic and nonagronomic uses, and methods of their use for controlling invertebrate pests such as nematodes in both agronomic and nonagronomic environments.

### BACKGROUND

The control of invertebrate pests is extremely important in achieving high crop efficiency. Damage by invertebrate pests to growing and stored agronomic crops can cause significant reduction in productivity and thereby result in increased costs to the consumer. The control of invertebrate pests in forestry, greenhouse crops, ornamentals, nursery crops, stored food and fiber products, livestock, household, turf, wood products, and public and animal health is also important. Many products are commercially available for these purposes, but the need continues for new compounds that are more effective, less costly, less toxic, environmentally safer or have different sites of action.

### SUMMARY

This disclosure is directed to compounds of Formula 1 (including all geometric and stereoisomers), N-oxides, and salts thereof, and compositions containing them and their use for controlling invertebrate pests:

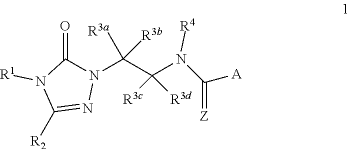

wherein
A is phenyl, pyridyl or pyrazinyl, each substituted with 1-3 R⁵, provided that at least one R⁵ is in the ortho position;
R¹ is C₁-C₆ alkyl, C₃-C₆ alkenyl, C₃-C₆ alkynyl, C₃-C₈ cycloalkyl or C₁-C₆ alkoxy, each R¹ optionally substituted with halogen, C₁-C₄ alkyl, C₁-C₄ haloalkyl, C₃-C₆ cycloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl; R₂ is C₁-C₃ haloalkyl or halogen;

### Z is O or S

R^{3a} and R^{3b} are each independently H, C₁-C₂ alkyl, C₁-C₂ haloalkyl, or R^{3a} and R^{3b} are taken together to form a 3- to 5-membered carbocyclic ring;
R^{3c} and R^{3d} are each independently H, C₁-C₂ alkyl, C₁-C₂ haloalkyl, or R^{3c} and R^{3d} are taken together to form a 3- to 5-membered carbocyclic ring;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;
with the proviso that when R¹ is Me, R² is not CF3.

This disclosure is also directed to a composition comprising compounds of Formula 1′ (including all geometric and stereoisomers), N-oxides, and salts thereof, and their use for controlling invertebrate pests:

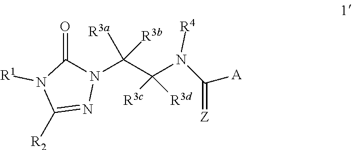

wherein
A is phenyl, pyridyl or pyrazinyl, each substituted with 1-3 R⁵, provided that at least one R⁵ is in the ortho position;
R¹ is C₁-C₆ alkyl, C₃-C₆ alkenyl, C₃-C₆ alkynyl, C₃-C₈ cycloalkyl or C₁-C₆ alkoxy, each R¹ optionally substituted with halogen, C₁-C₄ alkyl, C₁-C₄ haloalkyl, C₃-C₆ cycloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl; R₂ is C₁-C₃ haloalkyl or halogen;

### Z is O or S

R^{3a} and R^{3b} are each independently H, C₁-C₂ alkyl, C₁-C₂ haloalkyl, or R^{3a} and R^{3b} are taken together to form a 3- to 5-membered carbocyclic ring;
R^{3c} and R^{3d} are each independently H, C₁-C₂ alkyl, C₁-C₂ haloalkyl, or R^{3c} and R^{3d} are taken together to form a 3- to 5-membered carbocyclic ring;
R⁴ is H, C₁-C₄ alkyl, C₂-C₄ alkylcarbonyl or C₁-C₄ alkoxycarbonyl;
each R⁵ is independently halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl.

This disclosure also provides a composition comprising a compound of Formula 1 or Formula 1′, an N-oxide or a salt thereof, and at least one additional component selected from the group consisting of surfactants, solid diluents and liquid diluents. In one embodiment, this disclosure also provides a composition for controlling an invertebrate pest comprising a compound of Formula 1 or Formula 1′, an N-oxide or a salt thereof, and at least one additional component selected from the group consisting of surfactants, solid diluents and liquid diluents, said composition optionally further comprising at least one additional biologically active compound or agent.

This disclosure also provides a method for controlling an invertebrate pest comprising contacting the invertebrate pest or its environment with a biologically effective amount of a compound of Formula 1 or a compound of Formula 1′, an N-oxide or a salt thereof, (e.g., as a composition described herein). This disclosure also relates to such method wherein the invertebrate pest or its environment is contacted with a composition comprising a biologically effective amount of a compound of Formula 1 or Formula 1′, an N-oxide or a salt thereof, and at least one additional component selected from the group consisting of surfactants, solid diluents and liquid diluents, said composition optionally further comprising a biologically effective amount of at least one additional biologically active compound or agent.

This disclosure also provides a method for controlling an invertebrate pest comprising contacting the invertebrate pest or its environment with a biologically effective amount of any of the aforesaid compositions wherein the environment is a plant.

This disclosure also provides a method for controlling an invertebrate pest comprising contacting the invertebrate pest or its environment with a biologically effective amount of any of the aforesaid compositions wherein the environment is an animal.

This disclosure also provides a method for controlling an invertebrate pest comprising contacting the invertebrate pest or its environment with a biologically effective amount of any of the aforesaid compositions wherein the environment is a seed.

This disclosure also provides a method for protecting a seed from an invertebrate pest comprising contacting the seed with a biologically effective amount of a compound of Formula 1 or a compound of Formula 1′, an N-oxide or a salt thereof, (e.g., as a composition described herein). This disclosure also relates to the treated seed (i.e. seed contacted with a compound of Formula 1 or a compound of Formula 1′).

This disclosure also provides a method for increasing vigor of a crop plant comprising contacting the crop plant, the seed from which the crop plant is grown or the locus (e.g., growth medium) of the crop plant with a biologically effective amount of a compound of Formula 1 or a compound of Formula 1′ (e.g., as a composition described herein).

This disclosure further provides a method for protecting an animal from an invertebrate parasitic pest comprising administering to the animal a parasiticidally effective amount of a compound of Formula 1 or a compound of Formula 1′, an N-oxide or a salt thereof, (e.g., as a composition described herein). This disclosure also provides for the use of a compound of Formula 1 or a compound of Formula 1′, an N-oxide or a salt thereof, (e.g., as a composition described herein) in protecting an animal from an invertebrate pest.

### DETAILED DESCRIPTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed disclosure. The term “consisting essentially of” occupies a middle ground between “comprising” and “consisting of”.

Where applicants have defined an embodiment or a portion thereof with an open-ended term such as “comprising,” it should be readily understood that (unless otherwise stated) the description should be interpreted to also describe such an embodiment using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the disclosure are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods, nematodes and helminths of economic importance as pests. The term “arthropod” includes insects, mites, spiders, scorpions, centipedes, millipedes, pill bugs and symphylans. The term “gastropod” includes snails, slugs and other Stylommatophora. The term “nematode” includes members of the phylum Nematoda, such as phytophagous nematodes and helminth nematodes parasitizing animals. The term “helminth” includes all of the parasitic worms, such as roundworms (phylum Nematoda), heartworms (phylum Nematoda, class Secernentea), flukes (phylum Platyhelminthes, class Tematoda), acanthocephalans (phylum Acanthocephala), and tapeworms (phylum Platyhelminthes, class Cestoda).

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of maize or corn, soybeans and other legumes, rice, cereal (e.g., wheat, oats, barley, rye and rice), leafy vegetables (e.g., lettuce, cabbage, and other cole crops), fruiting vegetables (e.g., tomatoes, pepper, eggplant, crucifers and cucurbits), potatoes, other tuber vegetables, onions, garlic, sweet potatoes, grapes, cotton, tree fruits (e.g., pome, stone and citrus), small fruit (e.g., berries and cherries) and other specialty crops (e.g., canola, sunflower and olives).

The term “nonagronomic” refers to other than field crops, such as horticultural crops (e.g., greenhouse, nursery or ornamental plants not grown in a field), residential, agricultural, commercial and industrial structures, turf (e.g., sod farm, pasture, golf course, lawn, sports field, etc.), wood products, stored product, agro-forestry and vegetation management, public health (i.e. human) and animal health (e.g., domesticated animals such as pets, livestock and poultry, undomesticated animals such as wildlife) applications.

The term “crop vigor” refers to rate of growth or biomass accumulation of a crop plant. An “increase in vigor” refers to an increase in growth or biomass accumulation in a crop plant relative to an untreated control crop plant. The term “crop yield” refers to the return on crop material, in terms of both quantity and quality, obtained after harvesting a crop plant. An “increase in crop yield” refers to an increase in crop yield relative to an untreated control crop plant.

The term “biologically effective amount” refers to the amount of a biologically active compound (e.g., a compound of Formula 1 or a compound of Formula 1′) sufficient to produce the desired biological effect when applied to (i.e. contacted with) an invertebrate pest to be controlled or its environment, or to a plant, the seed from which the plant is grown, or the locus of the plant (e.g., growth medium) to protect the plant from injury by the invertebrate pest or for other desired effect (e.g., increasing plant vigor).

Nonagronomic applications include protecting an animal from an invertebrate parasitic pest by administering a parasiticidally effective (i.e. biologically effective) amount of a compound of the disclosure, typically in the form of a composition formulated for veterinary use, to the animal to be protected. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include necrosis, death, retarded growth, diminished mobility or lessened ability to remain on or in the host animal, reduced feeding and inhibition of reproduction. These effects on invertebrate parasite pests provide control (including prevention, reduction or elimination) of parasitic infestation or infection of the animal.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl. “Alkylene” denotes a straight-chain or branched alkanediyl. Examples of “alkylene” include CH₂, CH₂CH₂, CH(CH₃), CH₂CH₂CH₂, CH₂CH(CH₃) and the different butylene isomers. “Alkenylene” denotes a straight-chain or branched alkenediyl containing one olefinic bond. Examples of “alkenylene” include CH═CH, CH₂CH═CH, CH═C(CH₃) and the different butenylene isomers. “Alkynylene” denotes a straight-chain or branched alkynediyl containing one triple bond. Examples of “alkynylene” include C≡, CH₂C≡, C≡CCH₂ and the different butynylene isomers.

“Alkylamino” includes an NH radical substituted with straight-chain or branched alkyl. Examples of “alkylamino” include CH₃CH₂NH, CH₃CH₂CH₂NH and (CH₃)₂CHNH. Examples of “dialkylamino” include (CH₃)₂N, (CH₃CH₂)₂N and CH₃CH₂(CH₃)N. “Alkenylamine” includes an NH radical substituted with straight-chain or branched alkenes. Examples of “Alkenylamine” include CH₂═CHNH, CH₃CH═C(CH₃)CH₂NH, (CH₃)₂CHCH═CHNH and the different butenylamine, pentenylamine and hexenylamine isomers. “Alkynylamine” includes an NH radical substituted with straight-chain or branched alkynes. Examples of “Alkynylamine” include HC≡CNH, CH₃C≡CCH₂NH, (CH₃)₂CHC≡CNH and the different butynylamine, pentynylamine and hexynylamine isomers.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂OCH₂ and CH₃CH₂OCH₂CH₂.

“Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. Examples of “alkoxycarbonyl” include CH₃OC(═O)—, CH₃CH₂OC(═O)—, CH₃CH₂CH₂OC(═O)—, (CH₃)₂CHOC(═O)— and the different butoxycarbonyl, pentoxycarbonyl and hexoxycarbonyl isomers.

The term “alkylaminocarbonyl” denotes a straight-chain or branched alkylamino attached to and linked through a C(═O) group. Examples of “alkylaminocarbonyl” include CH₃NHC(═O), CH₃CH₂NHC(═O), CH₃CH₂CH₂NHC(═O) and (CH₃)₂CHNHC(═O). Examples of “dialkylaminocarbonyl” include (CH₃)₂NC(═O), (CH₃CH₂)₂NC(═O), CH₃CH₂(CH₃)NC(═O) and (CH₃)₂CH(CH₃)NC(═O). The term “alkenylaminocarbonyl” denotes a straight-chain or branched alkenylamino attached to and linked through a C(═O) group. Examples of “alkenylaminocarbonyl” include CH₂═CHNHC(═O), CH₃CH═C(CH₃)CH₂NHC(═O) and (CH₃)₂CHCH═CHNHC(═O). The term “alkynylaminocarbonyl” denotes a straight-chain or branched alkynylamino attached to and linked through a C(═O) group. Examples of “alkynylaminocarbonyl” include HC≡CNHC(═O), CH₃CH≡CCH₂NHC(═O) and (CH₃)₂CHC≡CNHC(═O).

“Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, cyclohexylmethyl and other cycloalkyl moieties bonded to straight-chain or branched alkyl groups. The term “cycloalkoxy” denotes cycloalkyl linked through an oxygen atom such as cyclopentyloxy and cyclohexyloxy. “Cycloalkylamino” denotes an NH radical substituted with cycloalkyl. Examples of “cycloalkylamino” include cyclopropylamino and cyclohexylamino. “Cycloalkylaminocarbonyl” denotes cycloalkylamino bonded to a C(═O) group, for example, cyclopentylaminocarbonyl and cyclohexylaminocarbonyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—. The terms “halocycloalkyl”, “haloalkoxy”, “haloalkylthio”, “haloalkylcarbonyl”, “haloalkoxycarbonyl”, “haloalkylaminocarbonyl”, “halodialkylaminocarbonyl” and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—.

The chemical abbreviations S(O) and S(═O) as used herein represent a sulfinyl moiety. The chemical abbreviations SO₂, S(O)₂ and S(═O)₂ as used herein represent a sulfonyl moiety. The chemical abbreviations C(O) and C(═O) as used herein represent a carbonyl moiety. The chemical abbreviations C(S) and C(═S) as used herein represent a thiocarbonyl moiety. The chemical abbreviations CO₂, C(O)O and C(═O)O as used herein represent an oxycarbonyl moiety. “CHO” means formyl.

The total number of carbon atoms in a substituent group is indicated by the “Cᵢ-Cⱼ” prefix where i and j are numbers from 1 to 9. For example, C₁-C₄ alkylsulfonyl designates methylsulfonyl through butylsulfonyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing a total of four carbon atoms, examples including CH₃CH₂CH₂OCH₂— and CH₃CH₂OCH₂CH₂—.

When a compound is substituted with a substituent bearing a subscript that indicates the number of said substituents can exceed 1, said substituents (when they exceed 1) are independently selected from the group of defined substituents, for example (R³)ₘ wherein m is 0, 1 or 2. When a group contains a substituent which can be hydrogen, for example R² or R¹², then when this substituent is taken as hydrogen, it is recognized that this is equivalent to said group being unsubstituted. When a variable group is shown to be optionally attached to a position, for example (R³)ₘ wherein m may be 0, then hydrogen may be at the position even if not recited in the variable group definition. When one or more positions on a group are said to be “not substituted” or “unsubstituted”, then hydrogen atoms are attached to take up any free valency.

Unless otherwise indicated, a “ring” or “ring system” as a component of Formula 1 or Formula 1′ is carbocyclic or heterocyclic. The term “ring system” denotes two or more fused rings. The terms “bicyclic ring system” and “fused bicyclic ring system” denote a ring system consisting of two fused rings, which can be “ortho-fused”, “bridged bicyclic” or “spirobicyclic”. An “ortho-fused bicyclic ring system” denotes a ring system wherein the two constituent rings have two adjacent atoms in common. A “bridged bicyclic ring system” is formed by bonding a segment of one or more atoms to nonadjacent ring members of a ring. A “spirobicyclic ring system” is formed by bonding a segment of two or more atoms to the same ring member of a ring. The term “fused heterobicyclic ring system” denotes a fused bicyclic ring system in which at least one ring atom is not carbon. The term “ring member” refers to an atom or other moiety (e.g., C(═O), C(═S), S(O) or S(O)₂) forming the backbone of a ring or ring system.

The terms “carbocyclic ring”, “carbocycle” or “carbocyclic ring system” denote a ring or ring system wherein the atoms forming the ring backbone are selected only from carbon. The terms “heterocyclic ring”, “heterocycle” or “heterocyclic ring system” denote a ring or ring system in which at least one atom forming the ring backbone is not carbon, e.g., nitrogen, oxygen or sulfur. Typically, a heterocyclic ring contains no more than 4 nitrogens, no more than 2 oxygens and no more than 2 sulfurs. Unless otherwise indicated, a carbocyclic ring or heterocyclic ring can be a saturated or unsaturated ring. “Saturated” refers to a ring having a backbone consisting of atoms linked to one another by single bonds; unless otherwise specified, the remaining atom valences are occupied by hydrogen atoms. Unless otherwise stated, an “unsaturated ring” may be partially unsaturated or fully unsaturated. The expression “fully unsaturated ring” means a ring of atoms in which the bonds between atoms in the ring are single or double bonds according to valence bond theory and furthermore the bonds between atoms in the ring include as many double bonds as possible without double bonds being cumulative (i.e. no C═C═C or C═C═N). The term “partially unsaturated ring” denotes a ring comprising at least one ring member bonded to an adjacent ring member through a double bond and which conceptually potentially accommodates a number of non-cumulated double bonds between adjacent ring members (i.e. in its fully unsaturated counterpart form) greater than the number of double bonds present (i.e. in its partially unsaturated form).

Unless otherwise indicated, heterocyclic rings and ring systems can be attached through any available carbon or nitrogen by replacement of a hydrogen on said carbon or nitrogen.

“Aromatic” indicates that each of the ring atoms is essentially in the same plane and has a p-orbital perpendicular to the ring plane, and in which (4n+2) π electrons, where n is a positive integer, are associated with the ring to comply with Hückel's rule. The term “aromatic ring system” denotes a carbocyclic or heterocyclic ring system in which at least one ring of the ring system is aromatic. When a fully unsaturated carbocyclic ring satisfies Hückel's rule, then said ring is also called an “aromatic ring” or “aromatic carbocyclic ring”.

The term “aromatic carbocyclic ring system” denotes a carbocyclic ring system in which at least one ring of the ring system is aromatic. When a fully unsaturated heterocyclic ring satisfies Hückel's rule, then said ring is also called a “heteroaromatic ring” or “aromatic heterocyclic ring”. The term “aromatic heterocyclic ring system” denotes a heterocyclic ring system in which at least one ring of the ring system is aromatic. The term “nonaromatic ring system” denotes a carbocyclic or heterocyclic ring system that may be fully saturated, as well as partially or fully unsaturated, provided that none of the rings in the ring system are aromatic. The term “nonaromatic carbocyclic ring system” denotes a carbocyclic ring in which no ring in the ring system is aromatic. The term “nonaromatic heterocyclic ring system” denotes a heterocyclic ring system in which no ring in the ring system is aromatic.

The term “optionally substituted” in connection with the heterocyclic rings refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

A wavy line or “-” in a structure fragment denotes the attachment point of the fragment to the remainder of the molecule.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this disclosure can exist as one or more stereoisomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. For a comprehensive discussion of all aspects of stereoisomerism, see Ernest L. Eliel and Samuel H. Wilen, Stereochemistry of Organic Compounds, John Wiley & Sons, 1994.

The compounds of the disclosure may be present as a mixture of stereoisomers or individual stereoisomers.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and 3-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus, a wide variety of salts of the compounds of Formula 1 or compounds of Formula 1′ are useful for control of invertebrate pests. The salts of the compounds of Formula 1 or compounds of Formula 1′ include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 or compounds of Formula 1′ contains an acidic moiety such as a carboxylic acid or phenol, salts also include those formed with organic or inorganic bases such as pyridine, triethylamine or ammonia, or amides, hydrides, hydroxides or carbonates of sodium, potassium, lithium, calcium, magnesium or barium. Accordingly, the present disclosure comprises compounds selected from Formula 1 or compounds of Formula 1′, N-oxides and suitable salts thereof.

Compounds selected from Formula 1 or compounds of Formula 1′, stereoisomers, tautomers, N-oxides, and salts thereof, typically exist in more than one form, and Formula 1 or Formula 1′ thus includes all crystalline and non-crystalline forms of the compounds that Formula 1 or compounds of Formula 1′, respectively, represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 or compounds of Formula 1′ can exhibit beneficial effects (e.g., suitability for preparation of useful formulations, improved biological performance) relative to another polymorph or a mixture of polymorphs of the same compound represented by Formula 1 or compounds of Formula 1′. Preparation and isolation of a particular polymorph of a compound represented by Formula 1 or Formula 1′ can be achieved by methods known to those skilled in the art including, for example, crystallization using selected solvents and temperatures. Compounds of this disclosure may exist as one or more crystalline polymorphs. This disclosure comprises both individual polymorphs and mixtures of polymorphs, including mixtures enriched in one polymorph relative to others. For a comprehensive discussion of polymorphism see R. Hilfiker, Ed., Polymorphism in the Pharmaceutical Industry, Wiley-VCH, Weinheim, 2006.

Embodiments of the present disclosure as described in the Summary include those described below. In the following Embodiments, Formula 1 or Formula 1′ include stereoisomers, N-oxides and salts thereof, and reference to “a compound of Formula 1” or “a compound of Formula 1′” includes the definitions of substituents specified in the Summary unless further defined in the Embodiments.

Embodiment 1. A compound of Formula 1 wherein A is

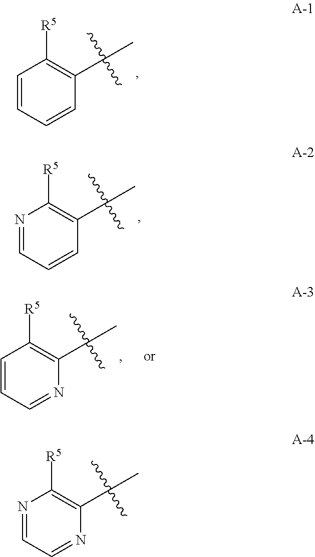

Embodiment 1a. A compound of Formula 1 or Embodiment 1 wherein A is A-1.
Embodiment 2. A compound of Formula 1 or any one of Embodiments 1 to 1a wherein R¹ is C₁-C₄ alkyl, C(Cl)F2, C₂-C₄ haloalkyl, C₃-C₄ cycloalkyl, or CH₂(C₃-C₄ cycloalkyl).
Embodiment 2b. A compound of Formula 1 or any of the preceding Embodiments wherein R¹ is t-Bu, C(Me)₂CF₃ or Me.
Embodiment 2c. A compound of Formula 1 or any of the preceding Embodiments wherein R¹ is t-Bu or Me.
Embodiment 3. A compound of Formula 1 or any of the preceding Embodiments wherein R² is CF₃, CF₂CF₃, CHF₂, or CF₂CF₂CF₃.
Embodiment 3a. A compound of Formula 1 or any of the preceding Embodiments wherein R² is CF₃.
Embodiment 4. A compound of Formula 1 or any of the preceding Embodiments wherein R^{3a}, R^{3b}, R^{3c} and R^{3d} are H.
Embodiment 5. A compound of Formula 1 or any of the preceding Embodiments wherein R⁴ is H.
Embodiment 6. A compound of Formula 1 or any of the preceding Embodiments wherein each R⁵ is independently CF₃, Cl, or Br.
Embodiment 7. A compound of Formula 1 or any of the preceding Embodiments wherein Z is O.
Embodiment 8. A composition comprising a compound of Formula 1′
wherein A is

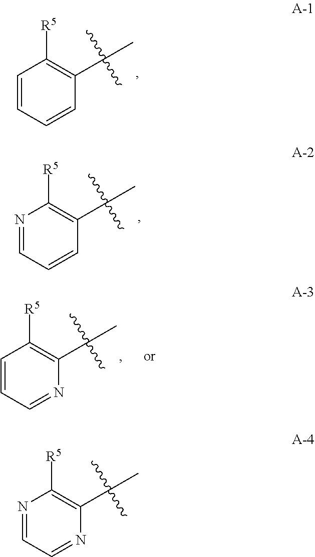

Embodiment 8a. A composition comprising a compound of Formula 1′ or Embodiment 8 wherein A is A-1.
Embodiment 9. A composition comprising a compound of Formula 1′ or any one of the preceding Embodiments wherein R¹ is C₁-C₄ alkyl, C₂-C₄ haloalkyl, C₃-C₄ cycloalkyl, or CH₂(C₃-C₄ cycloalkyl).
Embodiment 9a. A composition comprising a compound of Formula 1′ or any one of the preceding Embodiments wherein R¹ is t-Bu or Me.
Embodiment 9b. A composition comprising a compound of Formula 1′ or any one of the preceding Embodiments wherein R¹ is Me.
Embodiment 10. A composition comprising a compound of Formula 1′ or any one of the preceding Embodiments wherein R² is CF₃.
Embodiment 11. A composition comprising a compound of Formula 1′ or any one of the preceding Embodiments wherein R^{3a}, R^{3b}, R^{3c} and R^{3d} are H.
Embodiment 12. A composition comprising a compound of Formula 1′ or any one of the preceding Embodiments wherein R⁴ is H.
Embodiment 13. A composition comprising a compound of Formula 1′ or any one of the preceding Embodiments wherein each R⁵ is independently CF₃, Cl, or Br.
Embodiment 14. A composition comprising a compound of Formula 1′ or any one of the preceding Embodiments wherein Z is O.
Embodiments of this disclosure, including Embodiments 1-14 above as well as any other embodiments described herein, can be combined in any manner, and the descriptions of variables in the embodiments pertain not only to the compounds of Formula 1 or Formula 1′ but also to the starting compounds and intermediate compounds useful for preparing the compounds of Formula 1 or 1′. In addition, embodiments of this disclosure, including Embodiments 1-14 above as well as any other embodiments described herein, and any combination thereof, pertain to the compositions and methods of the present disclosure. Combinations of Embodiments 1-14 are illustrated by:
Embodiment A. A compound of Formula 1 wherein

### A is A1, A2, A3, or A4

R¹ is C₁-C₆ alkyl, or C₃-C₈ cycloalkyl, each R¹ optionally substituted with halogen, C₁-C₄ alkyl, C₁-C₄ haloalkyl, cyano, nitro, C₁-C₄ alkoxy, C₁-C₄ haloalkoxy, C₃-C₆ cycloalkyl, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;
R² is C₁-C₃ haloalkyl or halogen;

### Z is O or S

R^{3a} and R^{3b} are each independently H, C₁-C₂ alkyl, C₁-C₂haloalkyl, or R^{3a} and R^{3b} are taken together to form a 3- to 5-membered carbocyclic ring;
R^{3c} and R^{3d} are each independently H, C₁-C₂ alkyl, C₁-C₂ haloalkyl, or R^{3c} and R^{3d} are taken together to form a 3- to 5-membered carbocyclic ring;
R⁴ is H, C₁-C₄ alkyl, C₂-C₄ alkylcarbonyl or C₁-C₄ alkoxycarbonyl; and
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;
Embodiment B. A compound of Formula 1 or Embodiment A wherein

### A is A1 or A2;

R¹ is C₁-C₆ alkyl, or C₃-C₈ cycloalkyl, each R¹ optionally substituted with halogen, C₁-C₄ alkyl, C₁-C₄ haloalkyl, C₃-C₆ cycloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄haloalksulfinyl, C₁-C₄ alkylsulfonyl or C₁-C₄ haloalkylsulfonyl;
R² is C₁-C₃ haloalkyl or halogen;

### Z is O or S

R^{3a} and R^{3b} are each independently H, C₁-C₂ alkyl, C₁-C₂ haloalkyl, or R^{3a} and R^{3b} are taken together to form a 3- to 5-membered carbocyclic ring;
R^{3c} and R^{3d} are each independently H, C₁-C₂ alkyl, C₁-C₂ haloalkyl, or R^{3c} and R^{3d} are taken together to form a 3- to 5-membered carbocyclic ring;
R⁴ is H, C₁-C₄ alkyl, C₂-C₄ alkylcarbonyl or C₁-C₄ alkoxycarbonyl; and
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;
Embodiment C. A compound of Embodiment A or B wherein
R¹ is C₁-C₄ alkyl, C₂-C₄ haloalkyl, C₃-C₄ cycloalkyl, or CH₂(C₃-C₄ cycloalkyl); and

### Z is O.

Embodiment D. A compound of Embodiment C wherein
R² is C₁-C₃ haloalkyl.
Embodiment E. A compound of Embodiment D wherein
R^{3a}, R^{3b}, R^{3c} and R^{3d} are H;

### R⁴ is H.

Embodiment F. A compound of Embodiment E wherein each
R⁵ is independently CF₃, Cl, or Br.
Embodiment G. A compound of Embodiment F wherein
R¹ is t-Bu; and
R² is CF₃.
Embodiment H. A compound of Formula 1 wherein

### A is A-1;

R¹ is C₁-C₄ alkyl, C₂-C₄ haloalkyl, C₃-C₄ cycloalkyl, or CH₂(C₃-C₄ cycloalkyl).
R² is C₁-C₃ haloalkyl or halogen;
R^{3a} and R^{3b} are each independently H, C₁-C₂ alkyl, C₁-C₂ haloalkyl;
R^{3c} and R^{3d} are each independently H, C₁-C₂ alkyl, C₁-C₂ haloalkyl;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl; and

### Z is O.

Embodiment I. A compound of Formula 1 wherein

### A is A-1;

R¹ is t-Bu.
R² is C₁-C₃ haloalkyl or halogen;
R^{3a} and R^{3b} are each independently H, C₁-C₂ alkyl, C₁-C₂ haloalkyl;
R^{3c} and R^{3d} are each independently H, C₁-C₂ alkyl, C₁-C₂haloalkyl;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl; and Z is O.
Embodiment J. A compound of Formula 1 wherein

### A is A-1;

R¹ is t-Bu;
R² is CF₃;
R^{3a} and R^{3b} are each independently H, C₁-C₂ alkyl, C₁-C₂ haloalkyl;
R^{3c} and R^{3d} are each independently H, C₁-C₂ alkyl, C₁-C₂ haloalkyl;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl; and Z is O.
Embodiment K. A compound of Formula 1 wherein

### A is A-1;

R¹ is t-Bu.
R² is CF₃;
R^{3a}, R^{3b} are H;
R^{3c} and R^{3d} are each independently H, C₁-C₂ alkyl, C₁-C₂ haloalkyl;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄haloalkylsulfonyl; and

### Z is O.

Embodiment L. A compound of Formula 1 wherein

### A is A-1;

R¹ is t-Bu;
R² is CF₃;
R^{3a}, R^{3b} are H;
R^{3c} and R^{3d} are H;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl; and

### Z is O.

Embodiment M. A compound of Formula 1 wherein

### A is A-1;

R¹ is t-Bu;
R² is CF₃;
R^{3a}, R^{3b} are H;
R^{3c} and R^{3d} are H;

### R⁴ is H;

R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl; and

### Z is O.

Embodiment N. A compound of Formula 1 wherein

### A is A-1;

R¹ is t-Bu;
R² is CF₃;
R^{3a} and R^{3b} are H;
R^{3c} and R^{3d} are H;

### R⁴ is H;

each R⁵ is independently CF₃, Cl, or Br; and

### Z is O.

Embodiment AA. A composition comprising a compound of Formula 1′ wherein
A is A1, A2, A3, or A4, each substituted with 1-3 R⁵, provided that at least one R⁵ is in the ortho position;
R¹ is C₁-C₆ alkyl, or C₃-C₈ cycloalkyl, each R¹ optionally substituted with halogen, C₁-C₄ alkyl, C₁-C₄ haloalkyl, C₃-C₆ cycloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl C₁-C₄ haloalksulfinyl, C₁-C₄ alkylsulfonyl or C₁-C₄ haloalkylsulfonyl;
R² is C₁-C₃ haloalkyl or halogen;

### Z is O or S

R^{3a} and R^{3b} are each independently H, C₁-C₂ alkyl, C₁-C₂ haloalkyl, or R^{3a} and R^{3b} are taken together to form a 3- to 5-membered carbocyclic ring;
R^{3c} and R^{3d} are each independently H, C₁-C₂ alkyl, C₁-C₂haloalkyl, or R^{3c} and R^{3d} are taken together to form a 3- to 5-membered carbocyclic ring;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;
Embodiment BB. A composition comprising a compound of Formula 1′ or the composition of Embodiment AA wherein
A is A1, or A2 substituted with 1-3 R⁵, provided that at least one R⁵ is in the ortho position;
R¹ is C₁-C₆ alkyl, or C₃-C₈ cycloalkyl, each R¹ optionally substituted with halogen, C₁-C₄ alkyl, C₁-C₄ haloalkyl, C₃-C₆ cycloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;
R² is C₁-C₃ haloalkyl or halogen;

### Z is O or S

R^{3a} and R^{3b} are each independently H, C₁-C₂ alkyl, C₁-C₂ haloalkyl, or R^{3a} and R^{3b} are taken together to form a 3- to 5-membered carbocyclic ring;
R^{3c} and R^{3d} are each independently H, C₁-C₂ alkyl, C₁-C₂ haloalkyl, or R^{3c} and R^{3d} are taken together to form a 3- to 5-membered carbocyclic ring;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;
Embodiment CC. The composition of Embodiment AA or BB wherein
R¹ is C₁-C₄ alkyl, C₂-C₄ haloalkyl, C₃-C₄ cycloalkyl, or CH₂(C₃-C₄ cycloalkyl);

### Z is O.

Embodiment DD. The composition of Embodiment CC wherein
R² is C₁-C₃ haloalkyl.
Embodiment EE. The composition of Embodiment DD wherein
R^{3a}, R^{3b}, R^{3c} and R^{3d} are H;

### R⁴ is H.

Embodiment FF. The composition of Embodiment EE wherein each
R⁵ is independently CF₃, Cl, or Br.
Embodiment GG. The composition of Embodiment FF wherein
R¹ is t-Bu;
R² is CF₃.
Embodiment HH. A composition comprising a compound of Formula 1′ wherein

### A is A-1;

R¹ is C₁-C₄ alkyl, C₂-C₄ haloalkyl, C₃-C₄ cycloalkyl, or CH₂(C₃-C₄ cycloalkyl).
R² is C₁-C₃ haloalkyl or halogen;
R^{3a} and R^{3b} are each independently H, C₁-C₂ alkyl, C₁-C₂ haloalkyl;
R^{3c} and R^{3d} are each independently H, C₁-C₂ alkyl, C₁-C₂ haloalkyl;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;

### Z is O.

Embodiment II. A composition comprising a compound of Formula 1′ wherein

### A is A-1;

### R¹ is Me.

R² is C₁-C₃ haloalkyl or halogen;
R^{3a} and R^{3b} are each independently H, C₁-C₂ alkyl, C₁-C₂ haloalkyl;
R^{3c} and R^{3d} are each independently H, C₁-C₂ alkyl, C₁-C₂ haloalkyl;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;

### Z is O.

Embodiment JJ. A composition comprising a compound of Formula 1′ wherein

### A is A-1;

### R¹ is Me.

R² is CF₃;
R^{3a} and R^{3b} are each independently H, C₁-C₂ alkyl, C₁-C₂ haloalkyl;
R^{3c} and R^{3d} are each independently H, C₁-C₂ alkyl, C₁-C₂haloalkyl;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;

### Z is O.

Embodiment KK. A composition comprising a compound of Formula 1′ wherein

### A is A-1;

### R¹ is Me;

### R² is CF3;

R^{3a} and R^{3b} are H;
R^{3c} and R^{3d} are each independently H, C₁-C₂ alkyl, C₁-C₂haloalkyl;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;

### Z is O.

Embodiment LL. A composition comprising a compound of Formula 1′ wherein

### A is A-1;

### R¹ is Me;

R² is CF₃;
R^{3a} and R^{3b} are H;
R^{3c} and R^{3d} are H;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;

### Z is O.

Embodiment MM. A composition comprising a compound of Formula 1′ wherein

### A is A-1;

### R¹ is Me.

R² is CF₃;
R^{3a} and R^{3b} are H;
R^{3c} and R^{3d} are H;

### R⁴ is H;

R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;

### Z is O.

Embodiment NN. A composition comprising a compound of Formula 1′ wherein

### A is A-1;

### R¹ is Me;

R² is CF₃;
R^{3a} and R^{3b} are H;
R^{3c} and R^{3d} are H;

### R⁴ is H;

each R⁵ is independently CF₃, Cl, or Br;
Z is O. Embodiment X. A method for controlling an invertebrate pest comprising contacting the invertebrate pest or its environment with a biologically effective amount of a compound of Formula 1. Embodiment X1. The method of Claim X wherein the environment is soil or plant foliage.

Embodiments of this disclosure, including Embodiments 1-X1 above as well as any other embodiments described herein, can be combined in any manner, and the descriptions of variables in the embodiments pertain not only to the compounds of Formula 1 or Formula 1′ but also to the starting compounds and intermediate compounds useful for preparing the compounds of Formula 1 or Formula 1′. In addition, embodiments of this disclosure, including Embodiments 1-X1 above as well as any other embodiments described herein, and any combination thereof, pertain to the compositions and methods of the present disclosure. Specific embodiments include compounds of Formula 1 or Formula 1′ selected from the group consisting of:
N-[2-[4-(1,1-Dimethylethyl)-4,5-dihydro-5-oxo-3-(trifluoromethyl)-1H-1,2,4-triazol-1-yl]ethyl]-2-(trifluoromethyl)benzamide;
N-[2-[4,5-Dihydro-4-methyl-5-oxo-3-(trifluoromethyl)-1H-1,2,4-triazol-1-yl]ethyl]-2-(trifluoromethyl)benzamide;
N-[2-[4-(Cyclopropylmethyl)-4,5-dihydro-5-oxo-3-(trifluoromethyl)-1H-1,2,4-triazol-1-yl]ethyl]-2-(trifluoromethyl)benzamide;
N-[2-[4-(Cyclopropyl-4,5-dihydro-5-oxo-3-(trifluoromethyl)-1H-1,2,4-triazol-1-yl]ethyl]-2-(trifluoromethyl)benzamide;
2-Chloro-N-[2-[4-(1,1-dimethylethyl)-4,5-dihydro-5-oxo-3-(trifluoromethyl)-1H-1,2,4-triazol-1-yl]ethyl]benzamide;
2-Chloro-N-[2-[4-(1,1-dimethylethyl)-4,5-dihydro-5-oxo-3-(trifluoromethyl)-1H-1,2,4-triazol-1-yl]ethyl]-3-pyridinecarboxamide,
2-Bromo-N-[2-[4-(1,1-dimethylethyl)-4,5-dihydro-5-oxo-3-(trifluoromethyl)-1H-1,2,4-triazol-1-yl]ethyl]benzamide, and N-[2-[4,5-Dihydro-5-oxo-4-(2,2,2-trifluoro-1,1-dimethylethyl)-3-(trifluoromethyl)-1H-1,2,4-triazol-1-yl]ethyl]-2-(trifluoromethyl)benzamide.

TABLE 1

| *Compound | | Chemical Name & |
|---|---|---|
| No. | Compound Structure | Compound Number |
| 10 | 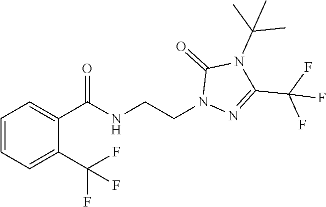 [SMILES: CC(C)(C)n1c(C(F)(F)F)nn(CCNC(=O)c2ccccc2C(F)(F)F)c1=O] | N-[2-[4-(1,1-Dimethylethyl)- 4,5-dihydro-5-oxo-3- (trifluoromethyl)-1H-1,2,4- triazol-1-yl]ethyl]-2- (trifluoromethyl)benzamide |
| 14 | 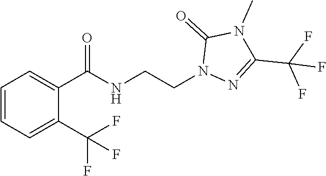 [SMILES: Cn1c(C(F)(F)F)nn(CCNC(=O)c2ccccc2C(F)(F)F)c1=O] | N-[2-[4,5-Dihydro-4-methyl-5- oxo-3-(trifluoromethyl)-1H- 1,2,4-triazol-1-yl]ethyl]-2- (trifluoromethyl)benzamide |
| 32 | 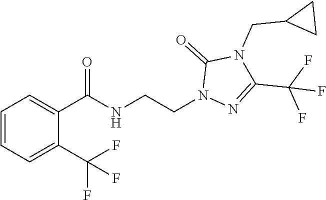 [SMILES: O=C(NCCn1nc(C(F)(F)F)n(CC2CC2)c1=O)c1ccccc1C(F)(F)F] | N-[2-[4-(Cyclopropylmethyl)- 4,5-dihydro-5-oxo-3- (trifluoromethyl)-1H-1,2,4- triazol-1-yl]ethyl]-2- (trifluoromethyl)benzamide |
| 2 | 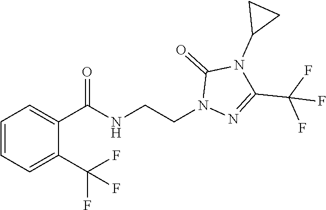 [SMILES: O=C(NCCn1nc(C(F)(F)F)n(C2CC2)c1=O)c1ccccc1C(F)(F)F] | N-[2-[4-(Cyclopropyl-4,5- dihydro-5-oxo-3- (trifluoromethyl)-1H-1,2,4- triazol-1-yl]ethyl]-2- (trifluoromethyl)benzamide |
| 38 | 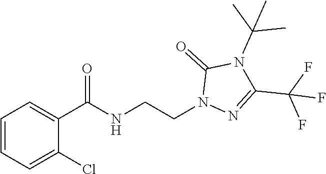 [SMILES: CC(C)(C)n1c(C(F)(F)F)nn(CCNC(=O)c2ccccc2Cl)c1=O] | 2-Chloro-N-[2-[4-(1,1- dimethylethyl)-4,5-dihydro-5- oxo-3-(trifluoromethyl)-1H- 1,2,4-triazol-1- yl]ethyl]benzamide |
| 92 | 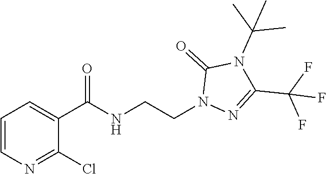 [SMILES: CC(C)(C)n1c(C(F)(F)F)nn(CCNC(=O)c2cccnc2Cl)c1=O] | 2-Chloro-N-[2-[4-(1,1- dimethylethyl)-4,5-dihydro-5- oxo-3-(trifluoromethyl)-1H- 1,2,4-triazol-1-yl]ethyl]-3- pyridinecarboxamide |
| 50 | 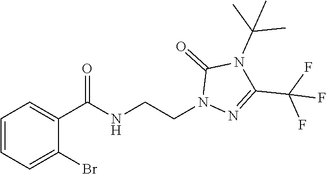 [SMILES: CC(C)(C)n1c(C(F)(F)F)nn(CCNC(=O)c2ccccc2Br)c1=O] | 2-Bromo-N-[2-[4-(1,1- dimethylethyl)-4,5-dihydro-5- oxo-3-(trifluoromethyl)-1H- 1,2,4-triazol-1- yl]ethyl]benzamide |
| 179 | 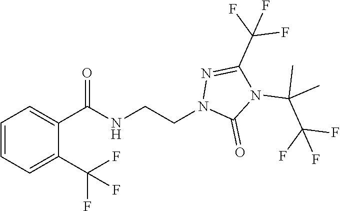 [SMILES: CC(C)(n1c(C(F)(F)F)nn(CCNC(=O)c2ccccc2C(F)(F)F)c1=O)C(F)(F)F] | N-[2-[4,5-Dihydro-5-oxo-4- (2,2,2-trifluoro-1,1- dimethylethyl)-3- (trifluoromethyl)-1H-1,2,4- triazol-1-yl]ethyl]-2- (trifluoromethyl)benzamide |

*Compound No. refers to the Compound Number as shown in Index Tables A-M.

Embodiment Y1. A composition comprising a compound of Formula 1 or Formula 1′ or any one of the preceding embodiments and at least one additional component selected from the group consisting of surfactants, solid diluents and liquid diluents, said composition optionally further comprising at least one additional biologically active compound or agent.

Embodiment Y2. The composition of embodiment Y1 wherein the at least one additional biologically active compound or agent is selected from the group consisting of abamectin, acephate, acequinocyl, acetamiprid, acrinathrin, afidopyropen, amidoflumet, amitraz, avermectin, azadirachtin, azinphos-methyl, benfuracarb, bensultap, bifenthrin, bifenazate, bistrifluron, borate, buprofezin, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyantraniliprole, cyclaniliprole, cyclobutrifluram, cycloprothrin, cycloxaprid, cyetpyrafen, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenitrothion, fenothiocarb, fenoxycarb, fenpropathrin, fenvalerate, fipronil, flometoquin, flonicamid, flubendiamide, flucythrinate, flufenerim, flufenoxuron, flufenoxystrobin, fluensulfone, fluopyram, flupentiofenox, flupyradifurone, fluvalinate, tau-fluvalinate, fonophos, formetanate, fosthiazate, halofenozide, heptafluthrin, hexaflumuron, hexythiazox, hydramethylnon, imidacloprid, indoxacarb, insecticidal soaps, isofenphos, lufenuron, malathion, meperfluthrin, metaflumizone, metaldehyde, methamidophos, methidathion, methiocarb, methomyl, methoprene, methoxychlor, methoxyfenozide, metofluthrin, monocrotophos, monofluorothrin, nicofluprole, nicotine, nitenpyram, nithiazine, novaluron, noviflumuron, N-[1,1-dimethyl-2-(methylthio)ethyl]-7-fluoro-2-(3-pyridinyl)-2H-indazole-4-carboxamide, N-[1,1-dimethyl-2-(methylsulfinyl)ethyl]-7-fluoro-2-(3-pyridinyl)-2H-indazole-4-carboxamide, N-[1,1-dimethyl-2-(methylsulfonyl)ethyl]-7-fluoro-2-(3-pyridinyl)-2H-indazole-4-carboxamide, N-(1-methylcyclopropyl)-2-(3-pyridinyl)-2H-indazole-4-carboxamide and N-[1-(difluoromethyl)cyclopropyl]-2-(3-pyridinyl)-2H-indazole-4-carboxamide, oxamyl, parathion, parathion-methyl, permethrin, phorate, phosalone, phosmet, phosphamidon, pirimicarb, profenofos, profluthrin, propargite, protrifenbute, pyflubumide, pymetrozine, pyrafluprole, pyrethrin, pyridaben, pyridalyl, pyrifluquinazon, pyriminostrobin, pyriprole, pyriproxyfen, rotenone, ryanodine, silafluofen, spinetoram, spinosad, spirodiclofen, spiromesifen, spirotetramat, sulprofos, sulfoxaflor, tebufenozide, tebufenpyrad, teflubenzuron, tefluthrin, tetrachlorvinphos, tetramethrin, tetramethylfluthrin, thiacloprid, thiamethoxam, thiodicarb, thiosultap-sodium, tioxazafen, tolfenpyrad, tralomethrin, triazamate, trichlorfon, triflumezopyrim, triflumuron, Bacillus thuringiensis delta-endotoxins, entomopathogenic bacteria, entomopathogenic viruses and entomopathogenic fungi.

Embodiment Y3. The composition of embodiment Y2 wherein the at least one additional biologically active compound or agent is selected from the group consisting of abamectin, acetamiprid, acrinathrin, afidopyropen, amitraz, avermectin, azadirachtin, benfuracarb, bensultap, bifenthrin, buprofezin, carbaryl, cartap, chlorantraniliprole, chlorfenapyr, chlorpyrifos, clothianidin, cyantraniliprole, cyclaniliprole, cycloprothrin, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenitrothion, fenothiocarb, fenoxycarb, fenvalerate, fipronil, flometoquin, flonicamid, flubendiamide, flufenoxuron, flufenoxystrobin, fluensulfone, flupiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiocarb, methomyl, methoprene, methoxyfenozide, metofluthrin, monofluorothrin, N-[1,1-dimethyl-2-(methylthio)ethyl]-7-fluoro-2-(3-pyridinyl)-2H-indazole-4-carboxamide, N-[1,1-dimethyl-2-(methylsulfinyl)ethyl]-7-fluoro-2-(3-pyridinyl)-2H-indazole-4-carboxamide, N-[1,1-dimethyl-2-(methylsulfonyl)ethyl]-7-fluoro-2-(3-pyridinyl)-2H-indazole-4-carboxamide, N-(1-methylcyclopropyl)-2-(3-pyridinyl)-2H-indazole-4-carboxamide and N-[1-(difluoromethyl)cyclopropyl]-2-(3-pyridinyl)-2H-indazole-4-carboxamide, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumezopyrim, triflumuron, Bacillus thuringiensis delta-endotoxins, all strains of Bacillus thuringiensis and all strains of nuclear polyhedrosis viruses.

Embodiment Y4. The composition of any one of embodiments Y1-Y3 further comprising a liquid fertilizer.

Embodiment Y5. The composition of Embodiment Y4 wherein the liquid fertilizer is aqueous-based.

Embodiment Y6. A soil drench formulation comprising the composition of any one of embodiments Y1-Y3.

Embodiment Y7. A spray composition comprising the composition of any one of embodiments Y1-Y3 and a propellant.

Embodiment Y8. A bait composition, comprising the composition of any one of embodiments Y1-Y3, one or more food materials, optionally an attractant, and optionally a humectant.

Embodiment Y9. A trap device for controlling an invertebrate pest, comprising: the bait composition of Embodiment Y8 and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

Embodiment Y10. A composition comprising the composition of any of Embodiments Y1-Y3 wherein the composition is a solid composition selected from dusts, powders, granules, pellets, prills, pastilles, tablets, and filled films.

Embodiment Y11. The composition of Embodiment Y10 wherein the composition is water-dispersible or water-soluble.

Embodiment Y12. A liquid or dry formulation comprising the composition of any one of Embodiments Y1-Y3 for use in a drip irrigation system, furrow during planting, handheld sprayer, backpack sprayer, boom sprayer, ground sprayer, aerial application, unmanned aerial vehicle, or a seed treatment.

Embodiment Y13. The liquid or dry formulation of Embodiment Y12 wherein said formulation is sprayed at an ultra-low volume.

Of note is that compounds of this disclosure are characterized by favorable metabolic and/or soil residual patterns and exhibit activity controlling a spectrum of agronomic and nonagronomic invertebrate pests.

Of particular note, for reasons of invertebrate pest control spectrum and economic importance, protection of agronomic crops from damage or injury caused by invertebrate pests by controlling invertebrate pests are embodiments of the disclosure. Compounds of this disclosure because of their favorable translocation properties or systemicity in plants also protect foliar or other plant parts which are not directly contacted with a compound of Formula 1 or a composition comprising the compound.

Also noteworthy as embodiments of the present disclosure are compositions comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent.

Further noteworthy as embodiments of the present disclosure are compositions for controlling an invertebrate pest comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent. Embodiments of the disclosure further include methods for controlling an invertebrate pest comprising contacting the invertebrate pest or its environment with a biologically effective amount of a compound of any of the preceding Embodiments (e.g., as a composition described herein).

Embodiments of the disclosure also include a composition comprising a compound of any of the preceding Embodiments, in the form of a soil drench liquid formulation. Embodiments of the disclosure further include methods for controlling an invertebrate pest comprising contacting the soil with a liquid composition as a soil drench comprising a biologically effective amount of a compound of any of the preceding Embodiments.

Embodiments of the disclosure also include a spray composition for controlling an invertebrate pest comprising a biologically effective amount of a compound of any of the preceding Embodiments and a propellant. Embodiments of the disclosure further include a bait composition for controlling an invertebrate pest comprising a biologically effective amount of a compound of any of the preceding Embodiments, one or more food materials, optionally an attractant, and optionally a humectant. Embodiments of the disclosure also include a device for controlling an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

Embodiments of the disclosure also include methods for protecting a seed from an invertebrate pest comprising contacting the seed with a biologically effective amount of a compound of any of the preceding Embodiments.

Embodiments of the disclosure also include methods for protecting an animal from an invertebrate parasitic pest comprising administering to the animal a parasiticidally effective amount of a compound of any of the preceding Embodiments.

Embodiments of the disclosure also include methods for controlling an invertebrate pest comprising contacting the invertebrate pest or its environment with a biologically effective amount of a compound of Formula 1, an N-oxide or a salt thereof, (e.g., as a composition described herein), provided that the methods are not methods of medical treatment of a human or animal body by therapy.

This disclosure also relates to such methods wherein the invertebrate pest or its environment is contacted with a composition comprising a biologically effective amount of a compound of Formula 1, an N-oxide or a salt thereof, and at least one additional component selected from the group consisting of surfactants, solid diluents and liquid diluents, said composition optionally further comprising a biologically effective amount of at least one additional biologically active compound or agent, provided that the methods are not methods of medical treatment of a human or animal body by therapy.

Embodiments of this disclosure also include use of an unmanned aerial vehicle (UAV) for the dispersion of the compositions disclosed herein over a planted area. In some embodiments the planted area is a crop-containing area. In some embodiments, the crop is selected from a monocot or dicot. In some embodiments, the crop is selected form rice, corn, barley, soybean, wheat, vegetable, tobacco, tea tree, fruit tree and sugar cane. In some embodiments, the compositions disclosed herein are formulated for spraying at an ultra-low volume. Products applied by drones may use water or oil as the spray carrier. Typical spray volume (including product) used for drone applications globally is 5.0 liters/ha-100 liters/ha (approximately 0.5-10 gpa). This includes the range of ultra-low spray volume (ULV) to low spray volume (LV). Although not common there may be situations where even lower spray volumes could be used as low as 1.0 liter/ha (0.1 gpa).

Compounds of Formula 1 can be prepared by one or more of the following methods and variations described in schemes 1-13. Substituents R1, R2, R3a, R3b, R3c, R3d, R4, Z and A in the compounds of formula 1-13 are defined in the summary of invention unless otherwise noted.

As shown in Scheme 1, a compound of Formula 1b (i.e. Formula 1 wherein Z is sulfur) may be prepared from a compound of Formula 1a (i.e. Formula 1 wherein Z is oxygen) by treatment with Lawesson's reagent, P₂S₅, or P₄S₁₀. Thioamide formation reactions of this type are typically conducted in an aprotic solvent such as toluene or 1,4-dioxane at elevated temperatures between 40° C. and the boiling point of the solvent. Reactions of this type are well known in the literature; see, for example, March and Smith, March's Advanced Organic Chemistry, 5^{th} ed., John Wiley & Sons, Inc., New York, 2001, Chapter 16.

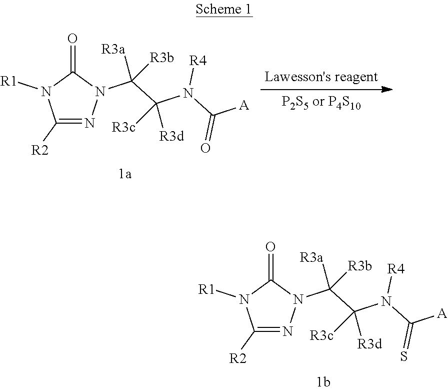

As shown in Scheme 2, compounds of Formula 1a (i.e. Formula 1 wherein Z is oxygen) can be prepared by acylation of amine derivatives of Formula 2 and acid chlorides of Formula 5, in a solvent such as dichloromethane or tetrahydrofuran in the presence of a base such as triethylamine, pyridine or N,N-diisopropylethylamine at ambient to the boiling point temperature of the solvent. These types of acylations are well documented in the literature. See for example March and Smith, March's Advanced Organic Chemistry, 5^{th} ed., John Wiley and Sons, Inc., New York, 2001, Chapter 10. Acid chlorides of formula 5 are commercially available or are prepared by methods well known to one skilled in the art of organic synthesis.

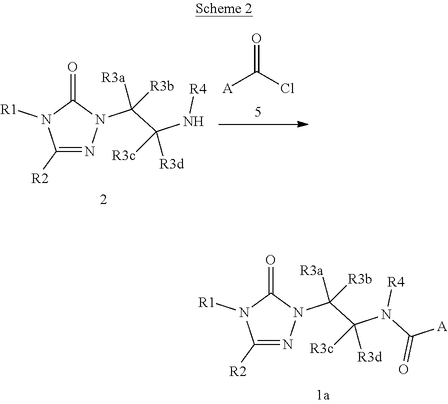

As shown in Scheme 3, compounds of Formula 1a (i.e. Formula 1 wherein Z is oxygen) can be prepared by acylation of amine derivatives of Formula 2 and commercially available acid derivatives of Formula 5a in a solvent such as dimethylformamide or dimethyl sulfoxide in the presence of a base such as triethylamine, pyridine or N, N-diisopropylethylamine at ambient temperature or heating at reflux temperature in the presence of HATU (1-[bis(dimethylamino)methylene]-1H-1,2,3-triazolo[4,5-b]pyridinium 3-oxide hexafluorophosphate) or EDC [1-ethyl-3-(3-dimethylaminopropyl)carbodiimide] and HOBT (hydroxy benzotriazole), optionally in the presence of an additive such as HOBT (1-hydroxy benzotriazole). These types of acylations are well documented in the literature. See for example March and Smith, March's Advanced Organic Chemistry, 5^{th} ed., John Wiley and Sons, Inc., New York, 2001, Chapter 10.

For an example of this reaction see step 7 of Example 1.

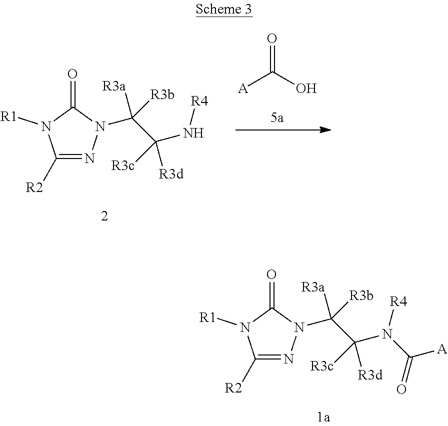

As shown in Scheme 4, compounds of Formula 1d (i.e. Formula 1 wherein Z is oxygen and R4 is other than H) can be prepared from Formula 1c (i.e. Formula 1 wherein Z is oxygen and R4 is H), by the reaction of compounds of Formula 7 in the presence of a base such as sodium hydride, potassium tert-butoxide, potassium carbonate or cesium carbonate in a solvent such as tetrahydrofuran, dimethyl formamide or dimethyl sulfoxide at temperatures below ambient to reflux temperature. These types of alkylations are well documented in the literature. See for example Journal of the American Chemical Society, 2019, 141(1), 159-162, Chemical Science, 2016, 7(3), 2229-2238.

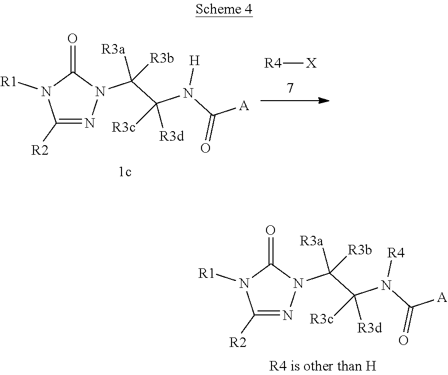

Compounds of Formula 1a (i.e. Formula 1 wherein Z is oxygen) can be also prepared by the method shown in Scheme 5. Triazolone derivatives of Formula 4 in a solvent such as tetrahydrofuran, acetonitrile, or dimethylformamide react with amides of Formula 3 having a leaving group X in the presence of a base such as sodium hydride, potassium tert-butoxide, lithium bis(trimethylsilylamide), triethylamine, or potassium carbonate, at temperatures from below ambient to the boiling point of the solvent.

For an example see step 9 of Example 1.

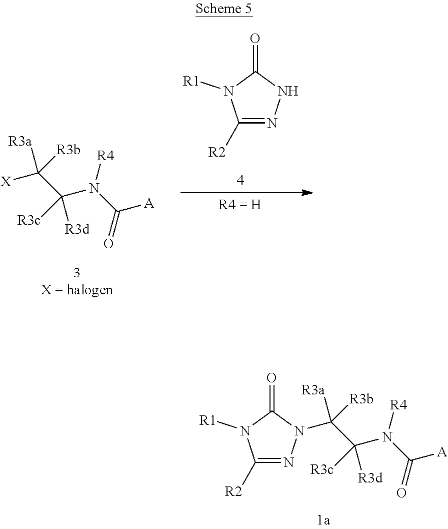

As shown in Scheme 6, compounds of Formula 9 can be prepared by a Mitsunobu reaction treating compounds of Formula 8 and triazolone compounds of Formula 4 in a solvent such as tetrahydrofuran, diethyether or dichloromethane in the presence of triphenyl phosphine and an azodicarboxylate, such as diethyl azodicarboxylate (DEAD) or diisopropyl azodicarboxylate (DIAD), in the presence of a base such as triethylamine or di-isopropyl amine at temperatures below ambient to the reflux temperature of the solvent. These types of Mitsunobu reactions are well documented in the literature. See for example Mitsunobu, O. (1981). “The Use of Diethyl Azodicarboxylate and Triphenylphosphine in Synthesis and Transformation of Natural Products”, Synthesis, 1981 (1), 1-28; Hughes, D. L. (1992). The Mitsunobu Reaction. Organic Reactions, 42. pp. 335-656, Swamy, K C. K.; Kumar, N. N. B.; Balaraman, E. & Kumar, K. V. P. P. (2009), “Mitsunobu and Related Reactions: Advances and Applications”, Chemical Reviews, 109 (6), 2551-2651.

For an example of this reaction see example step 5 of Example 1.

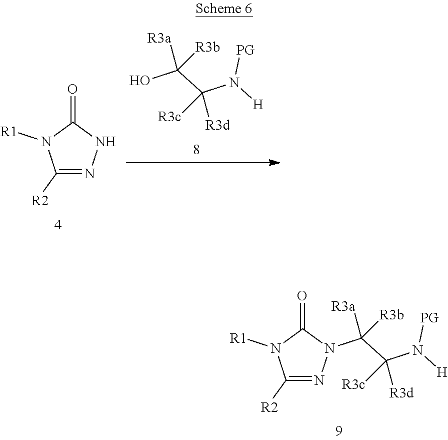

As shown in Scheme 7, N-deprotection of compounds of Formula 9 affords amines of Formula 2. Methods for N-deprotection can be found in Greene, Protective Groups in Organic Synthesis, John Willey and Sons, New York, 1981. For example, in Step 6 of Example 1, a tBOC (tert-butoxycarbonyl) is removed by reaction with hydrochloric acid in dioxane in a solvent such as methanol or ethanol.

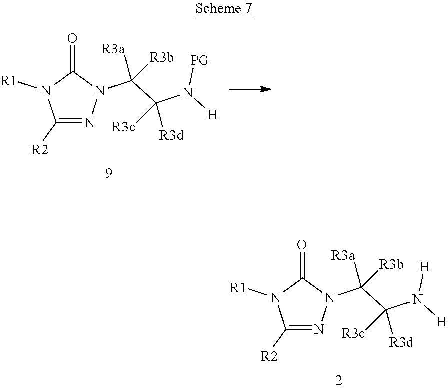

As shown in Scheme 8, protected amines of Formula 8 can be prepared from amines of Formula 6. Amines of Formula 6 are first N-protected with a protecting group PG such as tert-butoxy carbonyl or bezyloxycarbonyl to afford compounds of Formula 8. Methods for N-protection can be found in Greene, Protective Groups in Organic Synthesis, John Willey and Sons, New York, 1981.

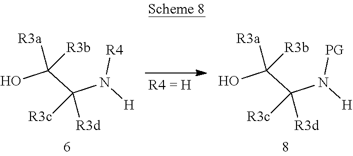

As shown in Scheme 9, compounds of Formula 3 can be prepared by treating an amine hydrochloride compound of Formula 10 in a solvent such as ethyl acetate, dichloromethane or tetrahydrofuran with an acid chloride of Formula 5 in presence of a base such as sodium hydrogen carbonate solution or triethylamine at ambient temperature. See, for example March and Smith, March's Advanced Organic Chemistry, 5^{th} ed., John Wiley and Sons, Inc., New York, 2001, Chapter 10. Acid chlorides of Formula 5 and amines of Formula 10 are commercially available or are prepared by methods well known to one skilled in the art of organic synthesis. For an example, see Step 8 of Example 1.

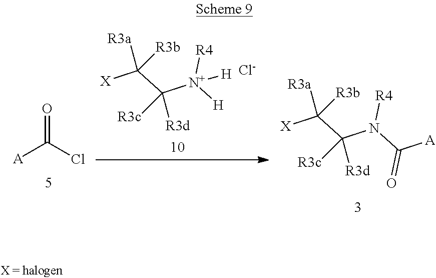

Triazolone compounds of Formula 4 can be prepared from compound of Formula 11 in the presence of a base such as sodium hydroxide, potassium hydroxide or potassium tert-butoxide in a solvent such as water, ethanol, 2-methyl-2-propanol or tetrahydrofuran at temperatures from ambient to the boiling point of the solvent. See for example see Eur. Pat. Appl., 726258, and Step 4 of Example 1.

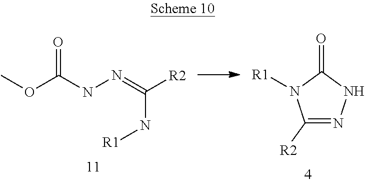

As shown in Scheme 11, compounds of Formula 11 can be prepared from compounds of Formula 12 by reaction with an amine of Formula R1NH2 in the presence of a solvent such as 1,4-dioxane, tetrahydrofuran or toluene. The reaction is carried out between room temperature to 40° C. See for example Eur. Pat. Appl., 726258, and Step 3 of Example 1.

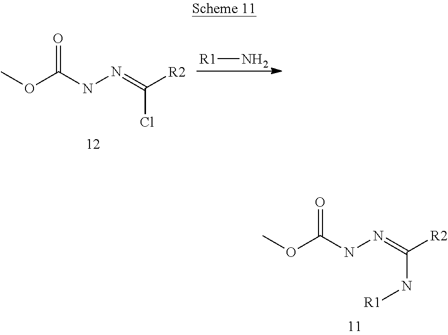

As shown in Scheme 12, compounds of Formula 13 can be prepared from compounds of Formula 12 by reaction with a sulphonyl chloride, such as methane-, ethane-, benzene- or p-toluene-sulphonyl chloride, where appropriate in the presence of an acid acceptor such as triethylamine or diisopropylethylamine, in a solvent such as dimethoxymethane, dioxane, tetrahydrofuran or methylene chloride at temperatures between 0° C. to 100° C. This can be found in Eur. Pat. Appl., 726258, and in Step 2 of Example 1.

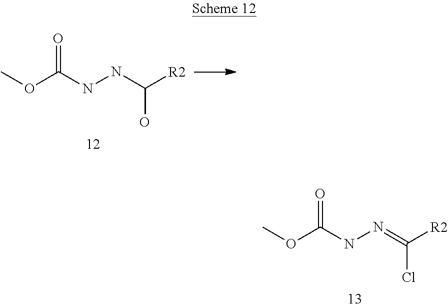

As shown in Scheme 13, the hydrazine carboxylic esters of Formula 12 can be prepared by known methods. See for example Synthesis 1991, 350-352. Compounds of Formula 14 and Formula 15 are commercially available or are prepared by methods well known to one skilled in the art of organic synthesis. For example, see Step 1 of Example 1.

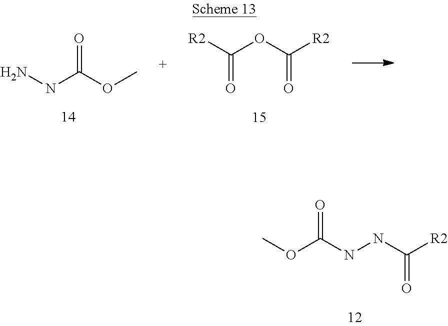

Schemes 1 through 13 illustrate methods to prepare compounds of Formula 1 having a variety of substituents. Compounds of Formula 1 having substituents other than those particularly noted for Schemes 1 through 13 can be prepared by general methods known in the art of synthetic organic chemistry, including methods analogous to those described for Schemes 1 to 13. Schemes 1 through 13 can similarly be used to prepare the Compounds of Formula 1′.

It is recognized by one skilled in the art that various functional groups can be converted into others to provide different compounds of Formula 1 or 1′. Compounds of Formula 1 or 1′, or intermediates for their preparation, may contain aromatic nitro groups, which can be reduced to amino groups, and then converted via reactions well-known in the art (e.g., Sandmeyer reaction) to various halides. By similar known reactions, aromatic amines (anilines) can be converted via diazonium salts to phenols, which can then be alkylated to prepare compounds of Formula 1 or 1′ with alkoxy substituents. Likewise, aromatic halides such as bromides or iodides prepared via the Sandmeyer reaction can react with alcohols under copper-catalyzed conditions, such as the Ullmann reaction or known modifications thereof, to provide compounds of Formula 1 or 1′ that contain alkoxy substituents. Additionally, some halogen groups, such as fluorine or chlorine, can be displaced with alcohols under basic conditions to provide compounds of Formula 1 or 1′ containing the corresponding alkoxy substituents. Compounds of Formula 1 or 1′ or precursors thereof containing a halide, preferably bromide or iodide, are particularly useful intermediates for transition metal-catalyzed cross-coupling reactions to prepare compounds of Formula 1 or Formula 1′. These types of reactions are well documented in the literature; see, for example, Tsuji in Transition Metal Reagents and Catalysts: Innovations in Organic Synthesis, John Wiley and Sons, Chichester, 2002; Tsuji in Palladium in Organic Synthesis, Springer, 2005; and Miyaura and Buchwald in Cross Coupling Reactions: A Practical Guide, 2002; and references cited therein.

It is recognized that some reagents and reaction conditions described above for preparing compounds of Formula 1 or Formula 1′ may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after introduction of the reagents depicted in the individual schemes, additional routine synthetic steps not described in detail may be needed to complete the synthesis of compounds of Formula 1 or Formula 1′. One skilled in the art will also recognize that it may be necessary to perform a combination of the steps illustrated in the above schemes in an order other than that implied by the particular sequence presented to prepare the compounds of Formula 1 or Formula 1′.

One skilled in the art will also recognize that compounds of Formula 1 or Formula 1′ and the intermediates described herein can be subjected to various electrophilic, nucleophilic, radical, organometallic, oxidation, and reduction reactions to add substituents or modify existing substituents.

### Synthesis Example 1

### Preparation of N-[2-[4-tert-butyl-5-oxo-3-(trifluoromethyl)-1,2,4-triazol-1-yl] ethyl]-2-(trifluoromethyl) benzamide (Compound 10)

N-[2-[4-tert-butyl-5-oxo-3-(trifluoromethyl)-1,2,4-triazol-1-yl] ethyl]-2-(trifluoromethyl) benzamide was prepared via two different routes as described below.

### Preparation of methyl N-[(2,2,2-trifluoroacetyl) amino] carbamate

Step 1: To a solution of methyl N-amino carbamate (10 g, 111.1 mmol) in diethyl ether trifluoro acetic anhydride (28 g, 133.3 mmol) was added at 0° C. and the resulting reaction mixture was stirred at room temperature for 4 h. Reaction was monitored by TLC. After completion of the reaction, diethyl ether was evaporated and co-distilled with toluene to obtain the compound methyl N-[(2,2,2-trifluoroacetyl) amino] carbamate as an off white solid (17 g, 85%). Crude compound was directly used for the next step.

1H NMR Spectra: (DMSO-d6, 500 MHz); δ-value in ppm

11.1 (m, 1H), 9.54 (m, 1H), 3.62 (s, 2H)

LC-MS: m/z 185 (M−1).

### Preparation of Methyl N—[(Z)-(1-chloro-2,2,2-trifluoro-ethylidene) amino] carbamate

Step 2: To a solution of methyl N-[(2,2,2-trifluoroacetyl) amino] carbamate (80 g, 430 mmol) in acetone (270 ml), benzene sulfonyl chloride (83.5 g, 473 mmol) and triethylamine (60 ml, 430 mmol) were added at room temperature and heated to 55° C. for 24 h. The reaction mixture was cooled to room temperature, filtered and washed with ethyl acetate (2×200 ml). The combined organic layer was washed with brine solution, dried over anhydrous sodium sulphate and concentrated under reduced pressure to afford compound N—[(Z)-(1-chloro-2,2,2-trifluoro-ethylidene) amino] carbamate (70 g, 79%). Crude compound was directly used for the next step. (M−1=204)

1H NMR (500 MHz, DMSO-d6) δ ppm 11.52 (s, 1H), 3.76 (s, 3H)

LC-MS: m/z 204 (M−1).

### Preparation of Methyl N—[(Z)-[1-(tert-butylamino)-2,2,2-trifluoro-ethylidene] amino]carbamate

Step 3: To a solution of methyl N—[(Z)-(1-chloro-2,2,2-trifluoro-ethylidene) amino] carbamate (3 g, 14.7 mmol) in 1,4-dioxane (15 ml), 2-methylpropan-2-amine (2.14 g, 29.4 mmol) was added slowly at r.t. over 5 min. The reaction mixture was allowed to stir at 40° C. for 4 h. Then it was poured into cold water (20 ml) and extracted with ethyl acetate (2×40 ml). The combined organic layer was washed with brine solution, dried over anhydrous sodium sulphate and concentrated under reduced pressure to afford off white solid compound methyl N—[(Z)-[1-(tert-butylamino)-2,2,2-trifluoro-ethylidene] amino] carbamate (2 g, 51%). Crude compound was directly used for the next step.

1H NMR (500 MHz, DMSO-d6) δ ppm 7.44 (bs, 1H), 3.85 (s, 3H), 1.46 (s, 9H)

LC-MS: m/z 242 (M+1).

### Preparation of 4-tert-butyl-3-(trifluoromethyl)-1H-1,2,4-triazol-5-one

Step 4: To an aqueous solution of sodium hydroxide (1.48 g, 37 mmol, in 20 ml water) methyl N—[(Z)-[1-(tert-butylamino)-2,2,2-trifluoro-ethylidene] amino] carbamate (3.0 g, 12.34 mmol) was added at room temperature. Then it was heated at 90° C. for 5 h. The reaction mixture was cooled to room temperature and neutralized with 1N HCl. The precipitated solid was filtered, washed with water (20 ml) and dried under reduced pressure to get the compound 4-tert-butyl-3-(trifluoromethyl)-1H-1,2,4-triazol-5-one as a yellow solid, which was further washed with pet ether. (1 g, 71%).

1H NMR (500 MHz, DMSO-d6) δ ppm 12.57 (m, 1H), 1.57 (s, 1H).

LC-MS: m/z 210 (M+1)

Route I:

### Preparation of tert-butyl N-[2-[4-tert-butyl-5-oxo-3-(trifluoromethyl)-1,2,4-triazol-1-yl]ethyl] carbamate

Step 5: To a solution of triphenyl phosphine (2.8 g, 10.76 mmol) in THF (30 ml), di-isopropyl azodicarboxylate (2.1 g, 10 mmol) was added slowly at 0° C. After 20 minutes, 4-tert-butyl-3-(trifluoromethyl)-1H-1,2,4-triazol-5-one (1.5 g, 7 mmol) and tert-butyl N-(2-hydroxyethyl) carbamate (7) (1.4 g, 8 mmol) were added followed by the addition of triethyl amine (1.5 g, 14 mmol) at room temperature over 5 mins. Then the reaction mixture was allowed to stir at r.t. for overnight. Reaction mixture was quenched with saturated bicarbonate solution (20 ml) and extracted with ethyl acetate (2×40 ml). The combined organic layer was washed with brine solution, dried over anhydrous sodium sulphate and concentrated under reduced pressure and purified by MPLC (40% ethyl acetate in pet ether) to get the desired compound tert-butyl N-[2-[4-tert-butyl-5-oxo-3-(trifluoromethyl)-1,2,4-triazol-1-yl] ethyl] carbamate (1.8 g, 72%).

¹H NMR spectra: (CDCl3, 500 MHz); δ-value in ppm

1H NMR (500 MHz, CHLOROFORM-d) δ ppm 3.93 (s, 1H), 3.3 (m, 1H), 1.67 (s, 5H), 1.42 (s, 8H).

LC-MS: m/z 354 (M+1)

### Preparation of 2-(2-aminoethyl)-4-tert-butyl-5-(trifluoromethyl)-1,2,4-triazol-3-one; hydrochloride

Step 6: To a solution of tert-butyl N-[2-[4-tert-butyl-5-oxo-3-(trifluoromethyl)-1,2,4-triazol-1-yl] ethyl] carbamate in methanol (1.8 g, 9 ml), 4M HCl in dioxane (9 ml) was added at room temperature. Then it was stirred at room temperature for overnight. The solvent was evaporated off and recrystallized from diethyl ether and pet ether to get the desired product as the hydrochloride salt 2-(2-aminoethyl)-4-tert-butyl-5-(trifluoromethyl)-1,2,4-triazol-3-one; hydrochloride (1 g, 71%).

1H NMR (500 MHz, DMSO-d6) δ ppm 8.19 (m, 1H), 4.0 (m, 1H), 3.06 (m, 1H), 1.59 (s, 3H).

LC-MS: m/z 254 (M+1)

### Preparation of N-[2-[4-tert-butyl-5-oxo-3-(trifluoromethyl)-1,2,4-triazol-1-yl] ethyl]-2-(trifluoromethyl) benzamide (Compound 10)

Step 7: To a solution of 2-(2-aminoethyl)-4-tert-butyl-5-(trifluoromethyl)-1,2,4-triazol-3-one; hydrochloride (200 mg, 692 mmol) in dichloromethane (10 ml), 2-(trifluoromethyl) benzoyl chloride (173 mg, 830 mmol) was added at room temperature followed by triethyl amine (140 mg, 1380 mmol). After stirring at room temperature for 2 h, the reaction mixture was quenched with saturated bicarbonate solution (20 ml) and extracted with ethyl acetate (2×40 ml). The combined organic layer was washed with brine solution, dried over anhydrous sodium sulphate and concentrated under reduced pressure and purified by MPLC (50% ethyl acetate in pet ether) to get the desired compound N-[2-[4-tert-butyl-5-oxo-3-(trifluoromethyl)-1,2,4-triazol-1-yl]ethyl]-2-(trifluoromethyl)benzamide (Compound 10) (200 mg, 68%).

1H NMR (500 MHz, CHLOROFORM-d) δ ppm 7.7 (m, 1H), 7.56 (s, 3H), 6.43 (m, 1H), 4.03 (m, 2H), 3.78 (m, 2H), 1.66 (s, 9H).

LC-MS: m/z 425 (M+1)

Route II:

### Preparation of N-(2-bromoethyl)-2-(trifluoromethyl) benzamide

Step 8: To a mixture of 2-bromoethylamine hydrochloride (4.7 g, 23 mmol) and saturated aqueous sodium hydrogen carbonate solution (50 mL) in ethyl acetate (35 mL), stirred and cooled in a bath of ice water, was added a solution of 2-(trifluoromethyl)benzoyl chloride (5.0 g, 24 mmol) in ethyl acetate (5 mL) over 1 h. A precipitate was observed. The mixture was stirred at ambient temperature for about 16 h. The mixture was washed with water, and the combined aqueous layers were extracted once with ethyl acetate. The organic phases were combined, dried over magnesium sulfate, and concentrated under reduced pressure. The resultant residue was triturated with hexanes with addition of small portions of 1-chlorobutane to obtain a white solid which was dried in air, to get the desired compound N-(2-bromoethyl)-2-(trifluoromethyl) benzamide (4.54 g, 67%).

¹H NMR (CDCl₃) [symbol] 7.72 (m, 1H), 7.6-7 (m, 3H), 6.25 (br s, 1H), 3.88 (m, 2H), 3.59 (m, 2H);

LC-MS: m/z 296 (M+1)

### Preparation of N-[2-[4-tert-butyl-5-oxo-3-(trifluoromethyl)-1,2,4-triazol-1-yl] ethyl]-2-(trifluoromethyl) benzamide

Step 9: To a mixture of 4-tert-butyl-3-(trifluoromethyl)-1H-1,2,4-triazol-5-one (prepared as in Step 4, 209 mg, 1.00 mmol) with anhydrous tetrahydrofuran (5 mL) was added a solution of potassium bis(trimethylsilyl)amide in anhydrous tetrahydrofuran (1.0 M commercial solution, 1.1 mL, 1.1 mmol) dropwise at ambient temperature. This mixture was stirred for 15 m, then a solution of N-(2-bromoethyl)-2-(trifluoromethyl) benzamide (prepared as above, 295 mg, 1.0 mmol) in anhydrous tetrahydrofuran (1 mL) was added and the mixture was heated at reflux temperature for 16 h. Anhydrous dimethylformamide (5 mL) was added, and heating was continued for 24 h at 100° C. At ambient temperature, sodium hydride (60% in mineral oil, 40 mg, 1.0 mmol) was added and the mixture was again heated at 100° C. 16 h. The mixture was allowed to cool, water was added, and this mixture was extracted with ethyl acetate. The organic phase was washed with water five times, dried over magnesium sulfate, and concentrated onto silica gel. The silica gel/product mixture was applied to a medium-pressure liquid chromatography silica gel column, and elution with a gradient of hexanes/ethyl acetate and concentration afforded the desired product N-[2-[4-tert-butyl-5-oxo-3-(trifluoromethyl)-1,2,4-triazol-1-yl] ethyl]-2-(trifluoromethyl) benzamide (Compound 10) (88 mg, 21%).

1H NMR (500 MHz, CHLOROFORM-d) δ ppm 7.7 (m, 1H), 7.56 (s, 3H), 6.43 (m, 1H), 4.03 (m, 2H), 3.78 (m, 2H), 1.66 (s, 9H).

LC-MS: m/z 425 (M+1)

By the procedures described herein together with methods known in the art, the following compounds of Tables 1a to 1j can be prepared. The following abbreviations are used in the Tables which follow: Me means methyl, OMe means methoxy, Et means ethyl, OEt means ethoxy, n-Pr means n-propyl, i-Pr means isopropyl, c-Pr means cyclopropyl, n-Bu means n-butyl, s-Bu means sec-butyl, t-Bu means tertiary butyl, c-Bu means cyclobutyl, Ph means phenyl and CN means cyano.

TABLE 1a

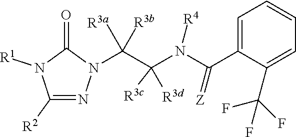

R^{3a-d} is H, R⁴ is H, Z is O

| R¹ | R² | R¹ | R² |
|---|---|---|---|
| Me | CF₃ | CH₂—CH₂—CF₃ | CF₂—CF₃ |
| Et | CF₃ | C(Me)₂—CF₃ | CF₂—CF₃ |
| i-Pr | CF₃ | CH₂—CHF₂—Me | CF₂—CF₃ |
| c-Pr | CF₃ | CH₂—c-Pr | CF₂—CF₃ |
| n-Pr | CF₃ | CH(Me)—c-Pr | CF₂—CF₃ |
| t-Bu | CF₃ | C(Me)₂—CH₂—CH₃ | CF₂—CF₃ |
| n-Bu | CF₃ | CH₂—i-Pr | CF₂—CF₃ |
| s-Bu | CF₃ | CH₂—t-Bu | CF₂—CF₃ |
| c-Bu | CF₃ | CH(Me)—t-Bu | CF₂—CF₃ |
| CH₂—CF₃ | CF₃ | C(Me)₂—t-Bu | CF₂—CF₃ |
| CH(Me)—CF₃ | CF₃ | Propargyl | CF₂—CF₃ |
| CPr—CF₃ | CF₃ | CH₂—CH═CH₂ | CF₂—CF₃ |
| CH₂—CH₂—CF₃ | CF₃ | CH(CH₂—CH₃)₂ | CF₂—CF₃ |
| C(Me)₂—CF₃ | CF₃ | CH₂—CH₂—OMe | CF₂—CF₃ |
| CH₂—CHF₂—Me | CF₃ | CH(Me)—CH₂—OMe | CF₂—CF₃ |
| CH₂—c-Pr | CF₃ | C(Me)₂—CH₂—OMe | CF₂—CF₃ |
| CH(Me)—c-Pr | CF₃ | CH₂—C(CH₂)Me | CF₂—CF₃ |
| C(Me)₂—CH₂—CH₃ | CF₃ | c-Pr(Me) | CF₂—CF₃ |
| CH₂—i-Pr | CF₃ | CH₂—CH₂—i-Pr | CF₂—CF₃ |
| CH₂—t-Bu | CF₃ | CH₂—CH₂—c-Pr | CF₂—CF₃ |
| CH(Me)—t-Bu | CF₃ | CH(Me)—CH(Me)₂ | CF₂—CF₃ |
| C(Me)₂—t-Bu | CF₃ | CH₂—CHF₂ | CF₂—CF₃ |
| Propargyl | CF₃ | Cyclopropyl | CF₂—CF3 |
| CH₂—CH═CH₂ | CF₃ | Cyclobutyl | CF₂—CF3 |
| CH(CH₂—CH₃)₂ | CF₃ | 1-(Trifluoromethyl)cyclopropyl | CF₂—CF3 |
| CH₂—CH₂—OMe | CF₃ | 1-(Trifluoromethyl)cyclobutyl | CF₂—CF3 |
| CH(Me)—CH₂—OMe | CF₃ | 3,3-Difluorocyclobutyl | CF₂—CF3 |
| C(Me)₂—CH₂—OMe | CF₃ | Cyclohexyl | CF₂—CF₃ |
| CH₂—C(CH₂)Me | CF₃ | Cyclopentyl | CF₂—CF₃ |
| c-Pr(Me) | CF₃ | CH₂—Cyclopropyl | CF₂—CF₃ |
| CH₂—CH₂—i-Pr | CF₃ | CH₂—Cyclobutyl | CF₂—CF₃ |
| CH₂—CH₂—c-Pr | CF₃ | CH₂—Cyclopentyl | CF₂—CF₃ |
| CH(Me)—CH(Me)₂ | CF₃ | CH₂—Cyclohexyl | CF₂—CF₃ |
| CH₂—CHF₂ | CF₃ | CH₂—CH₂—SMe | CF₂—CF₃ |
| Cyclopropyl | CF₃ | CH₂—CH₂—S(O)Me | CF₂—CF₃ |
| Cyclobutyl | CF₃ | CH₂—CH₂—SO₂Me | CF₂—CF₃ |
| 1-(Trifluoromethyl)cyclopropyl | CF₃ | C(Me)₂—CH₂—SMe | CF₂—CF₃ |
| 1-(Trifluoromethyl)cyclobutyl | CF₃ | C(Me)₂—CH₂—S(O)Me | CF₂—CF₃ |
| 3,3-Difluorocyclobutyl | CF₃ | C(Me)₂—CH₂—SO₂Me | CF₂—CF₃ |
| Cyclohexyl | CF₃ | CH₂—CF₂(Me) | CF₂—CF₃ |
| Cyclopentyl | CF₃ | CH₂—c-Pr(F₂) | CF₂—CF₃ |
| CH₂—Cyclopropyl | CF₃ | C(Me)₂—CO₂Me | CF₂—CF₃ |
| CH₂—Cyclobutyl | CF₃ | c-Pr—CO₂Me | CF₂—CF₃ |
| CH₂—Cyclopentyl | CF₃ | c-Pr—CHF₂ | CF₂—CF₃ |
| CH₂—Cyclohexyl | CF₃ | CH₂—CH(OMe)₂ | CF₂—CF₃ |
| CH₂—CH₂—SMe | CF₃ | C(Me)₂—CH₂—CN | CF₂—CF₃ |
| CH₂—CH₂—S(O)Me | CF₃ | CH₂—NO₂ | CF₂—CF₃ |
| CH₂—CH₂—SO₂Me | CF₃ | C(Me)₂—CH₂—tBu | CF₂—CF₃ |
| C(Me)₂—CH₂—SMe | CF₃ | C(Me)₂—Ph | CF₂—CF₃ |
| C(Me)₂—CH₂—S(O)Me | CF₃ | Me | CF₂—CF₂—CF₃ |
| C(Me)₂—CH₂—SO₂Me | CF₃ | Et | CF₂—CF₂—CF₃ |
| CH₂—CF₂(Me) | CF₃ | i-Pr | CF₂—CF₂—CF₃ |
| CH₂—c-Pr(F₂) | CF₃ | c-Pr | CF₂—CF₂—CF₃ |
| C(Me)₂—CO₂Me | CF₃ | n-Pr | CF₂—CF₂—CF₃ |
| c-Pr—CO₂Me | CF₃ | t-Bu | CF₂—CF₂—CF₃ |
| c-Pr—CHF₂ | CF₃ | n-Bu | CF₂—CF₂—CF₃ |
| CH₂—CH(OMe)₂ | CF₃ | s-Bu | CF₂—CF₂—CF₃ |
| C(Me)₂—CH₂—CN | CF₃ | c-Bu | CF₂—CF₂—CF₃ |
| CH₂—NO₂ | CF₃ | CH₂—CF₃ | CF₂—CF₂—CF₃ |
| C(Me)₂—CH₂—tBu | CF₃ | CH(Me)—CF₃ | CF₂—CF₂—CF₃ |
| C(Me)₂—Ph | CF₃ | CPr—CF₃ | CF₂—CF₂—CF₃ |
| Me | CHF₂ | CH₂—CH₂—CF₃ | CF₂—CF₂—CF₃ |
| Et | CHF₂ | C(Me)₂—CF₃ | CF₂—CF₂—CF₃ |
| i-Pr | CHF₂ | CH₂—CHF₂—Me | CF₂—CF₂—CF₃ |
| c-Pr | CHF₂ | CH₂—c-Pr | CF₂—CF₂—CF₃ |
| n-Pr | CHF₂ | CH(Me)—c-Pr | CF₂—CF₂—CF₃ |
| t-Bu | CHF₂ | C(Me)₂—CH₂—CH₃ | CF₂—CF₂—CF₃ |
| n-Bu | CHF₂ | CH₂—i-Pr | CF₂—CF₂—CF₃ |
| s-Bu | CHF₂ | CH₂—t-Bu | CF₂—CF₂—CF₃ |
| c-Bu | CHF₂ | CH(Me)—t-Bu | CF₂—CF₂—CF₃ |
| CH₂—CF₃ | CHF₂ | C(Me)₂—t-Bu | CF₂—CF₂—CF₃ |
| CH(Me)—CF₃ | CHF₂ | Propargyl | CF₂—CF₂—CF₃ |
| CPr—CF₃ | CHF₂ | CH₂—CH═CH₂ | CF₂—CF₂—CF₃ |
| CH₂—CH₂—CF₃ | CHF₂ | CH(CH₂—CH₃)₂ | CF₂—CF₂—CF₃ |
| C(Me)₂—CF₃ | CHF₂ | CH₂—CH₂—OMe | CF₂—CF₂—CF₃ |
| CH₂—CHF₂—Me | CHF₂ | CH(Me)—CH₂—OMe | CF₂—CF₂—CF₃ |
| CH₂—c-Pr | CHF₂ | C(Me)₂—CH₂—OMe | CF₂—CF₂—CF₃ |
| CH(Me)—c-Pr | CHF₂ | CH₂—C(CH₂)Me | CF₂—CF₂—CF₃ |
| C(Me)₂—CH₂—CH₃ | CHF₂ | c-Pr(Me) | CF₂—CF₂—CF₃ |
| CH₂—i-Pr | CHF₂ | CH₂—CH₂—i-Pr | CF₂—CF₂—CF₃ |
| CH₂—t-Bu | CHF₂ | CH₂—CH₂—c-Pr | CF₂—CF₂—CF₃ |
| CH(Me)—t-Bu | CHF₂ | CH(Me)—CH(Me)₂ | CF₂—CF₂—CF₃ |
| C(Me)₂—t-Bu | CHF₂ | CH₂—CHF₂ | CF₂—CF₂—CF₃ |
| Propargyl | CHF₂ | Cyclopropyl | CF₂—CF₂—CF₃ |
| CH₂—CH═CH₂ | CHF₂ | Cyclobutyl | CF₂—CF₂—CF₃ |
| CH(CH₂—CH₃)₂ | CHF₂ | 1-(Trifluoromethyl)cyclopropyl | CF₂—CF₂—CF₃ |
| CH₂—CH₂—OMe | CHF₂ | 1-(Trifluoromethyl)cyclobutyl | CF₂—CF₂—CF₃ |
| CH(Me)—CH₂—OMe | CHF₂ | 3,3-Difluorocyclobutyl | CF₂—CF₂—CF₃ |
| C(Me)₂—CH₂—OMe | CHF₂ | Cyclohexyl | CF₂—CF₂—CF₃ |
| CH₂—C(CH₂)Me | CHF₂ | Cyclopentyl | CF₂—CF₂—CF₃ |
| c-Pr(Me) | CHF₂ | CH₂—Cyclopropyl | CF₂—CF₂—CF₃ |
| CH₂—CH₂—i-Pr | CHF₂ | CH₂—Cyclobutyl | CF₂—CF₂—CF₃ |
| CH₂—CH₂—c-Pr | CHF₂ | CH₂—Cyclopentyl | CF₂—CF₂—CF₃ |
| CH(Me)—CH(Me)₂ | CHF₂ | CH₂—Cyclohexyl | CF₂—CF₂—CF₃ |
| CH₂—CHF₂ | CHF₂ | CH₂—CH₂—SMe | CF₂—CF₂—CF₃ |
| Cyclopropyl | CHF₂ | CH₂—CH₂—S(O)Me | CF₂—CF₂—CF₃ |
| Cyclobutyl | CHF₂ | CH₂—CH₂—SO₂Me | CF₂—CF₂—CF₃ |
| 1-(Trifluoromethyl)cyclopropyl | CHF₂ | C(Me)₂—CH₂—SMe | CF₂—CF₂—CF₃ |
| 1-(Trifluoromethyl)cyclobutyl | CHF₂ | C(Me)₂—CH₂—S(O)Me | CF₂—CF₂—CF₃ |
| 3,3-Difluorocyclobutyl | CHF₂ | C(Me)₂—CH₂—SO₂Me | CF₂—CF₂—CF₃ |
| Cyclohexyl | CHF₂ | CH₂—CF₂(Me) | CF₂—CF₂—CF₃ |
| Cyclopentyl | CHF₂ | CH₂—c-Pr(F₂) | CF₂—CF₂—CF₃ |
| CH₂—Cyclopropyl | CHF₂ | C(Me)₂—CO₂Me | CF₂—CF₂—CF₃ |
| CH₂—Cyclobutyl | CHF₂ | c-Pr—CO₂Me | CF₂—CF₂—CF₃ |
| CH₂—Cyclopentyl | CHF₂ | c-Pr—CHF₂ | CF₂—CF₂—CF₃ |
| CH₂—Cyclohexyl | CHF₂ | CH₂—CH(OMe)₂ | CF₂—CF₂—CF₃ |
| CH₂—CH₂—SMe | CHF₂ | C(Me)₂—CH₂—CN | CF₂—CF₂—CF₃ |
| CH₂—CH₂—S(O)Me | CHF₂ | CH₂—NO₂ | CF₂—CF₂—CF₃ |
| CH₂—CH₂—SO₂Me | CHF₂ | C(Me)₂—CH₂—tBu | CF₂—CF₂—CF₃ |
| C(Me)₂—CH₂—SMe | CHF₂ | C(Me)₂—Ph | CF₂—CF₂—CF₃ |
| C(Me)₂—CH₂—S(O)Me | CHF₂ | Me | Cl |
| C(Me)₂—CH₂—SO₂Me | CHF₂ | Et | Cl |
| CH₂—CF₂(Me) | CHF₂ | i-Pr | Cl |
| CH₂—c-Pr(F₂) | CHF₂ | c-Pr | Cl |
| C(Me)₂—CO₂Me | CHF₂ | n-Pr | Cl |
| c-Pr—CO₂Me | CHF₂ | t-Bu | Cl |
| c-Pr—CHF₂ | CHF₂ | n-Bu | Cl |
| CH₂—CH(OMe)₂ | CHF₂ | s-Bu | Cl |
| C(Me)₂—CH₂—CN | CHF₂ | c-Bu | Cl |
| CH₂—NO₂ | CHF₂ | CH₂—CF₃ | Cl |
| C(Me)₂—CH₂—tBu | CHF₂ | CH(Me)—CF₃ | Cl |
| C(Me)₂—Ph | CHF₂ | CPr—CF₃ | Cl |
| Me | C(Cl)F₂ | CH₂—CH₂—CF₃ | Cl |
| Et | C(Cl)F₂ | C(Me)₂—CF₃ | Cl |
| i-Pr | C(Cl)F₂ | CH₂—CHF₂—Me | Cl |
| c-Pr | C(Cl)F₂ | CH₂—c-Pr | Cl |
| n-Pr | C(Cl)F₂ | CH(Me)—c-Pr | Cl |
| t-Bu | C(Cl)F₂ | C(Me)₂—CH₂—CH₃ | Cl |
| n-Bu | C(Cl)F₂ | CH₂—i-Pr | Cl |
| s-Bu | C(Cl)F₂ | CH₂—t-Bu | Cl |
| c-Bu | C(Cl)F₂ | CH(Me)—t-Bu | Cl |
| CH₂—CF₃ | C(Cl)F₂ | C(Me)₂—t-Bu | Cl |
| CH(Me)—CF₃ | C(Cl)F₂ | Propargyl | Cl |
| CPr—CF₃ | C(Cl)F₂ | CH₂—CH═CH₂ | Cl |
| CH₂—CH₂—CF₃ | C(Cl)F₂ | CH(CH₂—CH₃)₂ | Cl |
| C(Me)₂—CF₃ | C(Cl)F₂ | CH₂—CH₂—OMe | Cl |
| CH₂—CHF₂—Me | C(Cl)F₂ | CH(Me)—CH₂—OMe | Cl |
| CH₂—c-Pr | C(Cl)F₂ | C(Me)₂—CH₂—OMe | Cl |
| CH(Me)—c-Pr | C(Cl)F₂ | CH₂—C(CH₂)Me | Cl |
| C(Me)₂—CH₂—CH₃ | C(Cl)F₂ | c-Pr(Me) | Cl |
| CH₂—i-Pr | C(Cl)F₂ | CH₂—CH₂—i-Pr | Cl |
| CH₂—t-Bu | C(Cl)F₂ | CH₂—CH₂—c-Pr | Cl |
| CH(Me)—t-Bu | C(Cl)F₂ | CH(Me)—CH(Me)₂ | Cl |
| C(Me)₂—t-Bu | C(Cl)F₂ | CH₂—CHF₂ | Cl |
| Propargyl | C(Cl)F₂ | Cyclopropyl | Cl |
| CH₂—CH═CH₂ | C(Cl)F₂ | Cyclobutyl | Cl |
| CH(CH₂—CH₃)₂ | C(Cl)F₂ | 1-(Trifluoromethyl)cyclopropyl | Cl |
| CH₂—CH₂—OMe | C(Cl)F₂ | 1-(Trifluoromethyl)cyclobutyl | Cl |
| CH(Me)—CH₂—OMe | C(Cl)F₂ | 3,3-Difluorocyclobutyl | Cl |
| C(Me)₂—CH₂—OMe | C(Cl)F₂ | Cyclohexyl | Cl |
| CH₂—C(CH₂)Me | C(Cl)F₂ | Cyclopentyl | Cl |
| c-Pr(Me) | C(Cl)F₂ | CH₂—Cyclopropyl | Cl |
| CH₂—CH₂—i-Pr | C(Cl)F₂ | CH₂—Cyclobutyl | Cl |
| CH₂—CH₂—c-Pr | C(Cl)F₂ | CH₂—Cyclopentyl | Cl |
| CH(Me)—CH(Me)₂ | C(Cl)F₂ | CH₂—Cyclohexyl | Cl |
| CH₂—CHF₂ | C(Cl)F₂ | CH₂—CH₂—SMe | Cl |
| Cyclopropyl | C(Cl)F₂ | CH₂—CH₂—S(O)Me | Cl |
| Cyclobutyl | C(Cl)F₂ | CH₂—CH₂—SO₂Me | Cl |
| 1-(Trifluoromethyl)cyclopropyl | C(Cl)F₂ | C(Me)₂—CH₂—SMe | Cl |
| 1-(Trifluoromethyl)cyclobutyl | C(Cl)F₂ | C(Me)₂—CH₂—S(O)Me | Cl |
| 3,3-Difluorocyclobutyl | C(Cl)F₂ | C(Me)₂—CH₂—SO₂Me | Cl |
| Cyclohexyl | C(Cl)F₂ | CH₂—CF₂(Me) | Cl |
| Cyclopentyl | C(Cl)F₂ | CH₂—c-Pr(F₂) | Cl |
| CH₂—Cyclopropyl | C(Cl)F₂ | C(Me)₂—CO₂Me | Cl |
| CH₂—Cyclobutyl | C(Cl)F₂ | c-Pr—CO₂Me | Cl |
| CH₂—Cyclopentyl | C(Cl)F₂ | c-Pr—CHF₂ | Cl |
| CH₂—Cyclohexyl | C(Cl)F₂ | CH₂—CH(OMe)₂ | Cl |
| CH₂—CH₂—SMe | C(Cl)F₂ | C(Me)₂—CH₂—CN | Cl |
| CH₂—CH₂—S(O)Me | C(Cl)F₂ | CH₂—NO₂ | Cl |
| CH₂—CH₂—SO₂Me | C(Cl)F₂ | C(Me)₂—CH₂—tBu | Cl |
| C(Me)₂—CH₂—SMe | C(Cl)F₂ | C(Me)₂—Ph | Cl |
| C(Me)₂—CH₂—S(O)Me | C(Cl)F₂ | Me | Br |
| C(Me)₂—CH₂—SO₂Me | C(Cl)F₂ | Et | Br |
| CH₂—CF₂(Me) | C(Cl)F₂ | i-Pr | Br |
| CH₂—c-Pr(F₂) | C(Cl)F₂ | c-Pr | Br |
| C(Me)₂—CO₂Me | C(Cl)F₂ | n-Pr | Br |
| c-Pr—CO₂Me | C(Cl)F₂ | t-Bu | Br |
| c-Pr—CHF₂ | C(Cl)F₂ | n-Bu | Br |
| CH₂—CH(OMe)₂ | C(Cl)F₂ | s-Bu | Br |
| C(Me)₂—CH₂—CN | C(Cl)F₂ | c-Bu | Br |
| CH₂—NO₂ | C(Cl)F₂ | CH₂—CF₃ | Br |
| C(Me)₂—CH₂—tBu | C(Cl)F₂ | CH(Me)—CF₃ | Br |
| C(Me)₂—Ph | C(Cl)F₂ | CPr—CF₃ | Br |
| Me | CF₂—CF₃ | CH₂—CH₂—CF₃ | Br |
| Et | CF₂—CF₃ | C(Me)₂—CF₃ | Br |
| i-Pr | CF₂—CF₃ | CH₂—CHF₂—Me | Br |
| c-Pr | CF₂—CF₃ | CH₂—c-Pr | Br |
| n-Pr | CF₂—CF₃ | CH(Me)—c-Pr | Br |
| t-Bu | CF₂—CF₃ | C(Me)₂—CH₂—CH₃ | Br |
| n-Bu | CF₂—CF₃ | CH2—i-Pr | Br |
| s-Bu | CF₂—CF₃ | CH2—t-Bu | Br |
| c-Bu | CF₂—CF₃ | CH(Me)—t-Bu | Br |
| CH₂—CF₃ | CF₂—CF₃ | C(Me)₂—t-Bu | Br |
| CH(Me)—CF₃ | CF₂—CF₃ | Propargyl | Br |
| CPr—CF₃ | CF₂—CF₃ | CH₂—CH═CH₂ | Br |
| CH(CH₂—CH₃)₂ | Br | CH2—Cyclopropyl | Br |
| CH₂—CH₂—OMe | Br | CH2—Cyclobutyl | Br |
| CH(Me)—CH₂—OMe | Br | CH2—Cyclopentyl | Br |
| C(Me)₂—CH₂—OMe | Br | CH2—Cyclohexyl | Br |
| CH₂—C(CH₂)Me | Br | CH2—CH2—SMe | Br |
| c-Pr(Me) | Br | CH2—CH2—S(O)Me | Br |
| CH₂—CH₂—i-Pr | Br | CH2—CH2—SO2Me | Br |
| CH₂—CH₂—c-Pr | Br | C(Me)₂—CH₂—SMe | Br |
| CH(Me)—CH(Me)₂ | Br | C(Me)₂—CH₂—S(O)Me | Br |
| CH₂—CHF₂ | Br | C(Me)₂—CH₂—SO₂Me | Br |
| Cyclopropyl | Br | CH2—CF2(Me) | Br |
| Cyclobutyl | Br | CH₂—c-Pr(F₂) | Br |
| 1-(Trifluoromethyl)cyclopropyl | Br | C(Me)₂—CO₂Me | Br |
| 1-(Trifluoromethyl)cyclobutyl | Br | c-Pr—CO₂Me | Br |
| 3,3-Difluorocyclobutyl | Br | c-Pr—CHF₂ | Br |
| Cyclohexyl | Br | CH₂—CH(OMe)₂ | Br |
| Cyclopentyl | Br | C(Me)₂—CH₂—CN | Br |
| C(Me)2—CH2—tBu | Br | CH₂—NO₂ | Br |
| C(Me)2—Ph | Br | | |

TABLE 1b

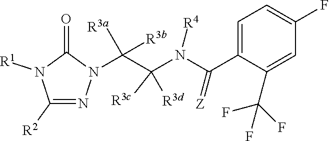

Table 1b is identical to Table 1a, except that the structure shown under the heading “Table 1a” is replaced by the structure shown above.

TABLE 1c

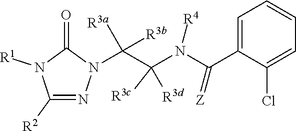

Table 1c is identical to Table 1a, except that the structure shown under the heading “Table 1a” is replaced by the structure shown above.

TABLE 1d

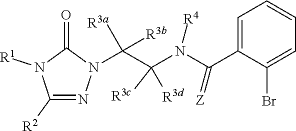

Table 1d is identical to Table 1a, except that the structure shown under the heading “Table 1a” is replaced by the structure shown above.

TABLE 1e

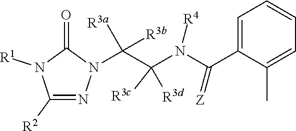

Table 1e is identical to Table 1a, except that the structure shown under the heading “Table 1a” is replaced by the structure shown above.

TABLE 1f

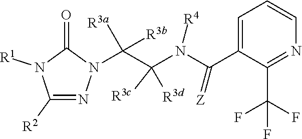

Table 1f is identical to Table 1a, except that the structure shown under the heading “Table 1a” is replaced by the structure shown above.

TABLE 1g

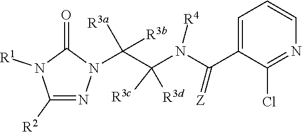

Table 1 g is identical to Table 1a, except that the structure shown under the heading “Table 1a” is replaced by the structure shown above.

TABLE 1h

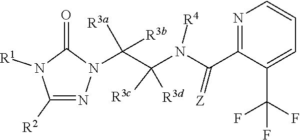

Table 1 h is identical to Table 1a, except that the structure shown under the heading “Table 1a” is replaced by the structure shown above.

TABLE 1i

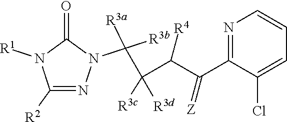

Table 1i is identical to Table 1a, except that the structure shown under the heading “Table 1a” is replaced by the structure shown above.

TABLE 1j

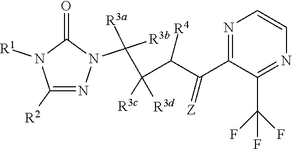

Table 1j is identical to Table 1a, except that the structure shown under the heading “Table 1a” is replaced by the structure shown above.

### Formulation/Utility

A compound of this disclosure will generally be used as an invertebrate pest control active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil in water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil in water emulsion, flowable concentrate and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively, the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

One way of dispensing the compositions disclosed herein over a target area, such as, but not limited to a crop-containing field, is by using drones. Use of drones or unmanned aerial vehicles (UAVs) in agricultural applications, such as for treating fields with chemical products, is rapidly expanding. A container of chemical products is coupled to the UAV and a material dispensing system mounted to the UAV, and the UAV is piloted above the area to be treated while the chemical product is dispensed.

The formulations will typically contain effective amounts of active ingredient, diluent and surfactant within the following approximate ranges which add up to 100 percent by weight.

### Weight Percent

| | Active | | |
|---|---|---|---|
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethylphosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters alkyl and aryl benzoates, γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present disclosure often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this disclosure may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 or Formula 1′ and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all formulations are prepared in conventional ways. Compound numbers refer to compounds in Index Tables A. Without further elaboration, it is believed that one skilled in the art using the preceding description can utilize the present disclosure to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 1 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 1 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 1 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

| | |
|---|---|
| Compound 1 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 1 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 1 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### Example H

Fertilizer Stick

| | |
|---|---|
| Compound 1 | 2.5% |
| pyrrolidone-styrene copolymer | 4.8% |
| tristyrylphenyl 16-ethoxylate | 2.3% |
| talc | 0.8% |
| corn starch | 5.0% |
| slow-release fertilizer | 36.0% |
| kaolin | 38.0% |
| water | 10.6% |

### Example I

Suspension Concentrate

| | |
|---|---|
| compound 1 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example J

Emulsion in Water

| | |
|---|---|
| compound 1 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example K

Oil Dispersion

| | |
|---|---|
| compound 1 | 25% |
| polyoxyethylene sorbitol hexaoleate | 15% |
| organically modified bentonite clay | 2.5% |
| fatty acid methyl ester | 57.5% |

### Example L

Suspoemulsion

| | |
|---|---|
| compound 1 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Compounds of this disclosure exhibit activity against a wide spectrum of invertebrate pests. These pests include invertebrates inhabiting a variety of environments such as, for example, plant foliage, roots, soil, harvested crops or other foodstuffs, building structures or animal integuments. These pests include, for example, invertebrates feeding on foliage (including leaves, stems, flowers and fruits), seeds, wood, textile fibers or animal blood or tissues, and thereby causing injury or damage to, for example, growing or stored agronomic crops, forests, greenhouse crops, ornamentals, nursery crops, stored foodstuffs or fiber products, or houses or other structures or their contents, or being harmful to animal health or public health. Those skilled in the art will appreciate that not all compounds are equally effective against all growth stages of all pests.

These present compounds and compositions are thus useful agronomically for protecting field crops from phytophagous invertebrate pests, and also nonagronomically for protecting other horticultural crops and plants from phytophagous invertebrate pests. This utility includes protecting crops and other plants (i.e. both agronomic and nonagronomic) that contain genetic material introduced by genetic engineering (i.e. transgenic) or modified by mutagenesis to provide advantageous traits. Examples of such traits include tolerance to herbicides, resistance to phytophagous pests (e.g., insects, mites, aphids, spiders, nematodes, snails, plant-pathogenic fungi, bacteria and viruses), improved plant growth, increased tolerance of adverse growing conditions such as high or low temperatures, low or high soil moisture, and high salinity, increased flowering or fruiting, greater harvest yields, more rapid maturation, higher quality and/or nutritional value of the harvested product, or improved storage or process properties of the harvested products. Transgenic plants can be modified to express multiple traits. Examples of plants containing traits provided by genetic engineering or mutagenesis include varieties of corn, cotton, soybean and potato expressing an insecticidal Bacillus thuringiensis toxin such as YIELD GARD®, KNOCKOUT®, STARLINK®, BOLLGARD®, NuCOTN® and NEWLEAF®, INVICTA RR2 PRO™, and herbicide-tolerant varieties of corn, cotton, soybean and rapeseed such as ROUNDUP READY®, LIBERTY LINK®, IMI®, STS® and CLEARFIELD®, as well as crops expressing N-acetyltransferase (GAT) to provide resistance to glyphosate herbicide, or crops containing the HRA gene providing resistance to herbicides inhibiting acetolactate synthase (ALS). The present compounds and compositions may exhibit enhanced effects with traits introduced by genetic engineering or modified by mutagenesis, thus enhancing phenotypic expression or effectiveness of the traits or increasing the invertebrate pest control effectiveness of the present compounds and compositions. In particular, the present compounds and compositions may exhibit enhanced effects with the phenotypic expression of proteins or other natural products toxic to invertebrate pests to provide greater-than-additive control of these pests.

Compositions of this disclosure can also optionally comprise plant nutrients, e.g., a fertilizer composition comprising at least one plant nutrient selected from nitrogen, phosphorus, potassium, sulfur, calcium, magnesium, iron, copper, boron, manganese, zinc, and molybdenum. Of note are compositions comprising at least one fertilizer composition comprising at least one plant nutrient selected from nitrogen, phosphorus, potassium, sulfur, calcium and magnesium. Compositions of the present disclosure which further comprise at least one plant nutrient can be in the form of liquids or solids. Of note are solid formulations in the form of granules, small sticks or tablets. Solid formulations comprising a fertilizer composition can be prepared by mixing the compound or composition of the present disclosure with the fertilizer composition together with formulating ingredients and then preparing the formulation by methods such as granulation or extrusion. Alternatively solid formulations can be prepared by spraying a solution or suspension of a compound or composition of the present disclosure in a volatile solvent onto a previous prepared fertilizer composition in the form of dimensionally stable mixtures, e.g., granules, small sticks or tablets, and then evaporating the solvent.

Nonagronomic uses refer to invertebrate pest control in the areas other than fields of crop plants. Nonagronomic uses of the present compounds and compositions include control of invertebrate pests in stored grains, beans and other foodstuffs, and in textiles such as clothing and carpets. Nonagronomic uses of the present compounds and compositions also include invertebrate pest control in ornamental plants, forests, in yards, along roadsides and railroad rights of way, and on turf such as lawns, golf courses and pastures. Nonagronomic uses of the present compounds and compositions also include invertebrate pest control in houses and other buildings which may be occupied by humans and/or companion, farm, ranch, zoo or other animals. Nonagronomic uses of the present compounds and compositions also include the control of pests such as termites that can damage wood or other structural materials used in buildings.

Nonagronomic uses of the present compounds and compositions also include protecting human and animal health by controlling invertebrate pests that are parasitic or transmit infectious diseases. The controlling of animal parasites includes controlling external parasites that are parasitic to the surface of the body of the host animal (e.g., shoulders, armpits, abdomen, inner part of the thighs) and internal parasites that are parasitic to the inside of the body of the host animal (e.g., stomach, intestine, lung, veins, under the skin, lymphatic tissue). External parasitic or disease transmitting pests include, for example, chiggers, ticks, lice, mosquitoes, flies, mites and fleas. Internal parasites include heartworms, hookworms and helminths. Compounds and compositions of the present disclosure are suitable for systemic and/or non-systemic control of infestation or infection by parasites on animals. Compounds and compositions of the present disclosure are particularly suitable for combating external parasitic or disease transmitting pests. Compounds and compositions of the present disclosure are suitable for combating parasites that infest agricultural working animals, such as cattle, sheep, goats, horses, pigs, donkeys, camels, buffalos, rabbits, hens, turkeys, ducks, geese and bees; pet animals and domestic animals such as dogs, cats, pet birds and aquarium fish; as well as so-called experimental animals, such as hamsters, guinea pigs, rats and mice. By combating these parasites, fatalities and performance reduction (in terms of meat, milk, wool, skins, eggs, honey, etc.) are reduced, so that applying a composition comprising a compound of the present disclosure allows more economic and simple husbandry of animals.

Examples of agronomic or nonagronomic invertebrate pests include eggs, larvae and adults of the order Lepidoptera, such as armyworms, cutworms, loopers, and heliothines in the family Noctuidae (e.g., pink stem borer (Sesamia inferens Walker), corn stalk borer (Sesamia nonagrioides Lefebvre), southern armyworm (Spodoptera eridania Cramer), fall armyworm (Spodoptera frugiperda J. E. Smith), beet armyworm (Spodoptera exigua Hübner), cotton leafworm (Spodoptera littoralis Boisduval), yellowstriped armyworm (Spodoptera ornithogalli Guenée), black cutworm (Agrotis ipsilon Hufnagel), velvetbean caterpillar (Anticarsia gemmatalis Hübner), green fruitworm (Lithophane antennata Walker), cabbage armyworm (Barathra brassicae Linnaeus), soybean looper (Pseudoplusia includens Walker), cabbage looper (Trichoplusia ni Hübner), tobacco budworm (Heliothis virescens Fabricius)); borers, casebearers, webworms, coneworms, cabbageworms and skeletonizers from the family Pyralidae (e.g., European corn borer (Ostrinia nubilalis Hübner), navel orangeworm (Amyelois transitella Walker), corn root webworm (Crambus caliginosellus Clemens), sod webworms (Pyralidae: Crambinae) such as sod worm (Herpetogramma licarsisalis Walker), sugarcane stem borer (Chilo infuscatellus Snellen), tomato small borer (Neoleucinodes elegantalis Guenée), green leafroller (Cnaphalocrocis medinalis), grape leaffolder (Desmia funeralis Hübner), melon worm (Diaphania nitidalis Stoll), cabbage center grub (Helluala hydralis Guenée), yellow stem borer (Scirpophaga incertulas Walker), early shoot borer (Scirpophaga infuscatellus Snellen), white stem borer (Scirpophaga innotata Walker), top shoot borer (Scirpophaga nivella Fabricius), dark-headed rice borer (Chilo polychrysus Meyrick), striped riceborer (Chilo suppressalis Walker), cabbage cluster caterpillar (Crocidolomia binotalis English)); leafrollers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck), citrus false codling moth (Cryptophlebia leucotreta Meyrick), citrus borer (Ecdytolopha aurantiana Lima), redbanded leafroller (Argyrotaenia velutinana Walker), obliquebanded leafroller (Choristoneura rosaceana Harris), light brown apple moth (Epiphyas postvittana Walker), European grape berry moth (Eupoecilia ambiguella Hübner), apple bud moth (Pandemis pyrusana Kearfott), omnivorous leafroller (Platynota stultana Walsingham), barred fruit-tree tortrix (Pandemis cerasana Hübner), apple brown tortrix (Pandemis heparana Denis & Schiffermuller)); and many other economically important lepidoptera (e.g., diamondback moth (Plutella xylostella Linnaeus), pink bollworm (Pectinophora gossypiella Saunders), gypsy moth (Lymantria dispar Linnaeus), peach fruit borer (Carposina niponensis Walsingham), peach twig borer (Anarsia lineatella Zeller), potato tuberworm (Phthorimaea operculella Zeller), spotted teniform leafminer (Lithocolletis blancardella Fabricius), Asiatic apple leafminer (Lithocolletis ringoniella Matsumura), rice leaffolder (Lerodea eufala Edwards), apple leafminer (Leucoptera scitella Zeller)); eggs, nymphs and adults of the order Blattodea including cockroaches from the families Blattellidae and Blattidae (e.g., oriental cockroach (Blatta orientalis Linnaeus), Asian cockroach (Blatella asahinai Mizukubo), German cockroach (Blattella germanica Linnaeus), brownbanded cockroach (Supella longipalpa Fabricius), American cockroach (Periplaneta americana Linnaeus), brown cockroach (Periplaneta brunnea Burmeister), Madeira cockroach (Leucophaea maderae Fabricius)), smoky brown cockroach (Periplaneta fuliginosa Service), Australian Cockroach (Periplaneta australasiae Fabr.), lobster cockroach (Nauphoeta cinerea Olivier) and smooth cockroach (Symploce pallens Stephens)); eggs, foliar feeding, fruit feeding, root feeding, seed feeding and vesicular tissue feeding larvae and adults of the order Coleoptera including weevils from the families Anthribidae, Bruchidae, and Curculionidae (e.g., boll weevil (Anthonomus grandis Boheman), rice water weevil (Lissorhoptrus oryzophilus Kuschel), granary weevil (Sitophilus granarius Linnaeus), rice weevil (Sitophilus oryzae Linnaeus)), annual bluegrass weevil (Listronotus maculicollis Dietz), bluegrass billbug (Sphenophorus parvulus Gyllenhal), hunting billbug (Sphenophorus venatus vestitus), Denver billbug (Sphenophorus cicatristriatus Fahraeus)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scarabaeidae (e.g., Japanese beetle (Popillia japonica Newman), oriental beetle (Anomala orientalis Waterhouse, Exomala orientalis (Waterhouse) Baraud), northern masked chafer (Cyclocephala borealis Arrow), southern masked chafer (Cyclocephala immaculata Olivier or C. lurida Bland), dung beetle and white grub (Aphodius spp.), black turfgrass ataenius (Ataenius spretulus Haldeman), green June beetle (Cotinis nitida Linnaeus), Asiatic garden beetle (Maladera castanea Arrow), May/June beetles (Phyllophaga spp.) and European chafer (Rhizotrogus majalis Razoumowsky)); carpet beetles from the family Dermestidae; wireworms from the family Elateridae; bark beetles from the family Scolytidae and flour beetles from the family Tenebrionidae.

In addition, agronomic and nonagronomic pests include: eggs, adults and larvae of the order Dermaptera including earwigs from the family Forficulidae (e.g., European earwig (Forficula auricularia Linnaeus), black earwig (Chelisoches morio Fabricius)); eggs, immatures, adults and nymphs of the order Hemiptera such as, plant bugs from the family Miridae, cicadas from the family Cicadidae, leafhoppers (e.g. Empoasca spp.) from the family Cicadellidae, bed bugs (e.g., Cimex lectularius Linnaeus) from the family Cimicidae, planthoppers from the families Fulgoridae and Delphacidae, treehoppers from the family Membracidae, psyllids from the families Liviidae, Psyllidae, and Triozidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, chinch bugs (e.g., hairy chinch bug (Blissus leucopterus hirtus Montandon) and southern chinch bug (Blissus insularis Barber)) and other seed bugs from the family Lygaeidae, spittlebugs from the family Cercopidae squash bugs from the family Coreidae, and red bugs and cotton stainers from the family Pyrrhocoridae.

Agronomic and nonagronomic pests also include: eggs, larvae, nymphs and adults of the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)); flat mites in the family Tenuipalpidae (e.g., citrus flat mite (Brevipalpus lewisi McGregor)); rust and bud mites in the family Eriophyidae and other foliar feeding mites and mites important in human and animal health, i.e. dust mites in the family Epidermoptidae, follicle mites in the family Demodicidae, grain mites in the family Glycyphagidae; ticks in the family Ixodidae, commonly known as hard ticks (e.g., deer tick (Ixodes scapularis Say), Australian paralysis tick (Ixodes holocyclus Neumann), American dog tick (Dermacentor variabilis Say), lone star tick (Amblyomma americanum Linnaeus)) and ticks in the family Argasidae, commonly known as soft ticks (e.g., relapsing fever tick (Ornithodoros turicata), common fowl tick (Argas radiatus)); scab and itch mites in the families Psoroptidae, Pyemotidae, and Sarcoptidae; eggs, adults and immatures of the order Orthoptera including grasshoppers, locusts and crickets (e.g., migratory grasshoppers (e.g., Melanoplus sanguinipes Fabricius, M. differentialis Thomas), American grasshoppers (e.g., Schistocerca americana Drury), desert locust (Schistocerca gregaria Forskal), migratory locust (Locusta migratoria Linnaeus), bush locust (Zonocerus spp.), house cricket (Acheta domesticus Linnaeus), mole crickets (e.g., tawny mole cricket (Scapteriscus vicinus Scudder) and southern mole cricket (Scapteriscus borellii Giglio-Tos)); eggs, adults and immatures of the order Diptera including leafminers (e.g., Liriomyza spp. such as serpentine vegetable leafminer (Liriomyza sativae Blanchard)), midges, fruit flies (Tephritidae), frit flies (e.g., Oscinella frit Linnaeus), soil maggots, house flies (e.g., Musca domestica Linnaeus), lesser house flies (e.g., Fannia canicularis Linnaeus, F. femoralis Stein), stable flies (e.g., Stomoxys calcitrans Linnaeus), face flies, horn flies, blow flies (e.g., Chrysomya spp., Phormia spp.), and other muscoid fly pests, horse flies (e.g., Tabanus spp.), bot flies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; eggs, adults and immatures of the order Thysanoptera including onion thrips (Thrips tabaci Lindeman), flower thrips (Frankliniella spp.), and other foliar feeding thrips; insect pests of the order Hymenoptera including ants of the Family Formicidae including the Florida carpenter ant (Camponotus floridanus Buckley), red carpenter ant (Camponotus ferrugineus Fabricius), black carpenter ant (Camponotus pennsylvanicus De Geer), white-footed ant (Technomyrmex albipes fr. Smith), big headed ants (Pheidole sp.), ghost ant (Tapinoma melanocephalum Fabricius); Pharaoh ant (Monomorium pharaonis Linnaeus), little fire ant (Wasmannia auropunctata Roger), fire ant (Solenopsis geminata Fabricius), red imported fire ant (Solenopsis invicta Buren), Argentine ant (Iridomyrmex humilis Mayr), crazy ant (Paratrechina longicornis Latreille), pavement ant (Tetramorium caespitum Linnaeus), cornfield ant (Lasius alienus Farster) and odorous house ant (Tapinoma sessile Say). Other Hymenoptera including bees (including carpenter bees), hornets, yellow jackets, wasps, and sawflies (Neodiprion spp.; Cephus spp.); insect pests of the order Isoptera including termites in the Termitidae (e.g., Macrotermes sp., Odontotermes obesus Rambur), Kalotermitidae (e.g., Cryptotermes sp.), and Rhinotermitidae (e.g., Reticulitermes sp., Coptotermes sp., Heterotermes tenuis Hagen) families, the eastern subterranean termite (Reticulitermes flavipes Kollar), western subterranean termite (Reticulitermes hesperus Banks), Formosan subterranean termite (Coptotermes formosanus Shiraki), West Indian drywood termite (Incisitermes immigrans Snyder), powder post termite (Cryptotermes brevis Walker), drywood termite (Incisitermes snyderi Light), southeastern subterranean termite (Reticulitermes virginicus Banks), western drywood termite (Incisitermes minor Hagen), arboreal termites such as Nasutitermes sp. and other termites of economic importance; insect pests of the order Thysanura such as silverfish (Lepisma saccharina Linnaeus) and firebrat (Thermobia domestica Packard); insect pests of the order Mallophaga and including the head louse (Pediculus humanus capitis De Geer), body louse (Pediculus humanus Linnaeus), chicken body louse (Menacanthus stramineus Nitszch), dog biting louse (Trichodectes canis De Geer), fluff louse (Goniocotes gallinae De Geer), sheep body louse (Bovicola ovis Schrank), short-nosed cattle louse (Haematopinus eurysternus Nitzsch), long-nosed cattle louse (Linognathus vituli Linnaeus) and other sucking and chewing parasitic lice that attack man and animals; insect pests of the order Siphonoptera including the oriental rat flea (Xenopsylla cheopis Rothschild), cat flea (Ctenocephalides felis Bouche), dog flea (Ctenocephalides canis Curtis), hen flea (Ceratophyllus gallinae Schrank), sticktight flea (Echidnophaga gallinacea Westwood), human flea (Pulex irritans Linnaeus) and other fleas afflicting mammals and birds. Additional arthropod pests covered include: spiders in the order Araneae such as the brown recluse spider (Loxosceles reclusa Gertsch & Mulaik) and the black widow spider (Latrodectus mactans Fabricius), and centipedes in the order Scutigeromorpha such as the house centipede (Scutigera coleoptrata Linnaeus).

Examples of invertebrate pests of stored grain include larger grain borer (Prostephanus truncatus), lesser grain borer (Rhyzopertha dominica), rice weevil (Stiophilus oryzae), maize weevil (Stiophilus zeamais), cowpea weevil (Callosobruchus maculatus), red flour beetle (Tribolium castaneum), granary weevil (Stiophilus granarius), Indian meal moth (Plodia interpunctella), Mediterranean flour beetle (Ephestia kuhniella) and flat or rusty grain beetle (Cryptolestis ferrugineus).

Compounds of the present disclosure may have activity on members of the Classes Nematoda, Cestoda, Trematoda, and Acanthocephala including economically important members of the orders Strongylida, Ascaridida, Oxyurida, Rhabditida, Spirurida, and Enoplida such as but not limited to economically important agricultural pests (i.e. root knot nematodes in the genus Meloidogyne, lesion nematodes in the genus Pratylenchus, stubby root nematodes in the genus Trichodorus, etc.) and animal and human health pests (i.e. all economically important flukes, tapeworms, and roundworms, such as Strongylus vulgaris in horses, Toxocara canis in dogs, Haemonchus contortus in sheep, Dirofilaria immitis Leidy in dogs, Anoplocephala perfoliata in horses, Fasciola hepatica Linnaeus in ruminants, etc.).

Compounds of the disclosure show particularly high activity against pests in the order Lepidoptera (e.g., Alabama argillacea Hübner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenée (rice leaf roller), Crambus caliginosellus Clemens (corn root webworm), Crambus teterrellus Zincken (bluegrass webworm), Cydia pomonella Linnaeus (codling moth), Earias insulana Boisduval (spiny bollworm), Earias vittella Fabricius (spotted bollworm), Helicoverpa armigera Hübner (American bollworm), Helicoverpa zea Boddie (corn earworm), Heliothis virescens Fabricius (tobacco budworm), Herpetogramma licarsisalis Walker (sod webworm), Lobesia botrana Denis & Schiffermuller (grape berry moth), Pectinophora gossypiella Saunders (pink bollworm), Phyllocnistis citrella Stainton (citrus leafminer), Pieris brassicae Linnaeus (large white butterfly), Pieris rapae Linnaeus (small white butterfly), Plutella xylostella Linnaeus (diamondback moth), Spodoptera exigua Hübner (beet armyworm), Spodoptera litura Fabricius (tobacco cutworm, cluster caterpillar), Spodoptera frugiperda J. E. Smith (fall armyworm), Trichoplusia ni Hübner (cabbage looper) and Tuta absoluta Meyrick (tomato leafminer)).

Compounds of the disclosure may also have significant activity on members from the order Hemiptera including: Acyrthosiphon pisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Passerini (rosy apple aphid), Eriosoma lanigerum Hausmann (woolly apple aphid), Hyalopterus pruni Geoffroy (mealy plum aphid), Lipaphis pseudobrassicae Davis (turnip aphid), Metopolophium dirhodum Walker (rose-grain aphid), Macrosiphum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graminum Rondani (greenbug), Sitobion avenae Fabricius (English grain aphid), Therioaphis maculata Buckton (spotted alfalfa aphid), Toxoptera aurantii Boyer de Fonscolombe (black citrus aphid), and Toxoptera citricidus Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifoihi Bellows & Perring (silverleaf whitefly), Dialeurodes citri Ashmead (citrus whitefly) and Trialeurodes vaporariorum Westwood (greenhouse whitefly); Empoasca fabae Harris (potato leafhopper), Laodelphax striatellus Fallen (smaller brown planthopper), Macrosteles quadrilineatus Forbes (aster leafhopper), Nephotettix cincticeps Uhler (green rice leafhopper), Nephotettix nigropictus Stål (rice leafhopper), Nilaparvata lugens Stål (brown planthopper), Peregrinus maidis Ashmead (corn planthopper), Sogatella furcifera Horvath (white-backed planthopper), Tagosodes orizicolus Muir (rice delphacid), Typhlocyba pomaria McAtee (white apple leafhopper), Erythroneura spp. (grape leafhoppers); Magicidada septendecim Linnaeus (periodical cicada); Icerya purchasi Maskell (cottony cushion scale), Quadraspidiotus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persimmon psylla).

Compounds of this disclosure also have activity on members from the order Hemiptera including: Acrosternum hilare Say (green stink bug), Anasa tristis De Geer (squash bug), Blissus leucopterus leucopterus Say (chinch bug), Cimex lectularius Linnaeus (bed bug) Corythuca gossypii Fabricius (cotton lace bug), Cyrtopeltis modesta Distant (tomato bug), Dysdercus suturellus Herrich-Schäffer (cotton stainer), Euchistus servus Say (brown stink bug), Euchistus variolarius Palisot de Beauvois (one-spotted stink bug), Graptosthetus spp. (complex of seed bugs), Halymorpha halys Stål (brown marmorated stink bug), Leptoglossus corculus Say (leaf-footed pine seed bug), Lygus lineolaris Palisot de Beauvois (tarnished plant bug), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper). Other insect orders controlled by compounds of the disclosure include Thysanoptera (e.g., Frankliniella occidentalis Pergande (western flower thrips), Scirthothrips citri Moulton (citrus thrips), Sericothrips variabilis Beach (soybean thrips), and Thrips tabaci Lindeman (onion thrips); and the order Coleoptera (e.g., Leptinotarsa decemlineata Say (Colorado potato beetle), Epilachna varivestis Mulsant (Mexican bean beetle) and wireworms of the genera Agriotes, Athous or Limonius).

Of note is use of compounds of this disclosure for controlling western flower thrip (Frankliniella occidentalis). Of note is use of compounds of this disclosure for controlling diamondback moth (Plutella xylostella). Of note is use of compounds of this disclosure for controlling fall armyworm (Spodoptera frugiperda).

Compounds of the present disclosure are also useful for increasing vigor of a crop plant. This method comprises contacting the crop plant (e.g., foliage, flowers, fruit or roots) or the seed from which the crop plant is grown with a compound of Formula 1 in amount sufficient to achieve the desired plant vigor effect (i.e. biologically effective amount). Typically, the compound of Formula 1 is applied in a formulated composition. Although the compound of Formula 1 is often applied directly to the crop plant or its seed, it can also be applied to the locus of the crop plant, i.e. the environment of the crop plant, particularly the portion of the environment in close enough proximity to allow the compound of Formula 1 to migrate to the crop plant. The locus relevant to this method most commonly comprises the growth medium (i.e. medium providing nutrients to the plant), typically soil in which the plant is grown. Treatment of a crop plant to increase vigor of the crop plant thus comprises contacting the crop plant, the seed from which the crop plant is grown or the locus of the crop plant with a biologically effective amount of a compound of Formula 1.

Increased crop vigor can result in one or more of the following observed effects: (a) optimal crop establishment as demonstrated by excellent seed germination, crop emergence and crop stand; (b) enhanced crop growth as demonstrated by rapid and robust leaf growth (e.g., measured by leaf area index), plant height, number of tillers (e.g., for rice), root mass and overall dry weight of vegetative mass of the crop; (c) improved crop yields, as demonstrated by time to flowering, duration of flowering, number of flowers, total biomass accumulation (i.e. yield quantity) and/or fruit or grain grade marketability of produce (i.e. yield quality); (d) enhanced ability of the crop to withstand or prevent plant disease infections and arthropod, nematode or mollusk pest infestations; and (e) increased ability of the crop to withstand environmental stresses such as exposure to thermal extremes, suboptimal moisture or phytotoxic chemicals.

The compounds of the present disclosure can increase the vigor of treated plants compared to untreated plants by killing or otherwise preventing feeding of phytophagous invertebrate pests in the environment of the plants. In the absence of such control of phytophagous invertebrate pests, the pests reduce plant vigor by consuming plant tissues or sap, or transmitting plant pathogens such as viruses. Even in the absence of phytophagous invertebrate pests, the compounds of the disclosure may increase plant vigor by modifying metabolism of plants. Generally, the vigor of a crop plant will be most significantly increased by treating the plant with a compound of the disclosure if the plant is grown in a nonideal environment, i.e. an environment comprising one or more aspects adverse to the plant achieving the full genetic potential it would exhibit in an ideal environment.

Of note is the present method for increasing vigor of a crop plant wherein the crop plant is grown in an environment comprising phytophagous invertebrate pests. Also of note is the present method for increasing vigor of a crop plant wherein the crop plant is grown in an environment not comprising phytophagous invertebrate pests. Also of note is the present method for increasing vigor of a crop plant wherein the crop plant is grown in an environment comprising an amount of moisture less than ideal for supporting growth of the crop plant. Of note is the present method for increasing vigor of a crop plant wherein the crop is rice. Also of note is the present method for increasing vigor of a crop plant wherein the crop is maize (corn). Also, of note is the present method for increasing vigor of a crop plant wherein the crop is soybean.

Compounds of this disclosure can also be mixed with one or more other biologically active compounds or agents including insecticides, fungicides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, other biologically active compounds or entomopathogenic bacteria, virus or fungi to form a multi-component pesticide giving an even broader spectrum of agronomic and nonagronomic utility. Thus, the present disclosure also pertains to a composition comprising a biologically effective amount of a compound of Formula 1, at least one additional component selected from the group consisting of surfactants, solid diluents and liquid diluents, and at least one additional biologically active compound or agent. For mixtures of the present disclosure, the other biologically active compounds or agents can be formulated together with the present compounds, including the compounds of Formula 1, to form a premix, or the other biologically active compounds or agents can be formulated separately from the present compounds, including the compounds of Formula 1, and the two formulations combined together before application (e.g., in a spray tank) or, alternatively, applied in succession.

Examples of such biologically active compounds or agents with which compounds of this disclosure can be formulated are insecticides such as abamectin, acephate, acequinocyl, acetamiprid, acrinathrin, acynonapyr, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11H-naphtho[2,1-b]pyrano[3,4-e]pyran-4-yl]methyl cyclopropanecarboxylate), amidoflumet, amitraz, avermectin, azadirachtin, azinphos-methyl, benfuracarb, bensultap, benzpyrimoxan, bifenthrin, kappa-bifenthrin, bifenazate, bistrifluron, borate, broflanilide, buprofezin, cadusafos, carbaryl, carbofuran, cartap, carzol, chlorantraniliprole, chlorfenapyr, chlorfluazuron, chloroprallethrin, chlorpyrifos, chlorpyrifos-e, chlorpyrifos-methyl, chromafenozide, clofentezin, chloroprallethrin, clothianidin, cyantraniliprole, (3-bromo-1-(3-chloro-2-pyridinyl)-N-[4-cyano-2-methyl-6-[(methylamino)carbonyl]phenyl]-1H-pyrazole-5-carboxamide), cyclaniliprole (3-bromo-N-[2-bromo-4-chloro-6-[[(1-cyclopropylethyl)amino]carbonyl]phenyl]-1-(3-chloro-2-pyridinyl)-1H-pyrazole-5-carboxamide), cyclobutrifluram, cycloprothrin, cycloxaprid ((5S,8R)-1-[(6-chloro-3-pyridinyl)methyl]-2,3,5,6,7,8-hexahydro-9-nitro-5,8-Epoxy-1H-imidazo[1,2-a]azepine), cyenopyrafen, cyetpyrafen, cyflumetofen, cyfluthrin, beta-cyfluthrin, cyhalodiamide, cyhalothrin, gamma-cyhalothrin, lambda-cyhalothrin, cypermethrin, alpha-cypermethrin, zeta-cypermethrin, cyromazine, deltamethrin, diafenthiuron, diazinon, dicloromesotiaz, dieldrin, diflubenzuron, dimefluthrin, dimehypo, dimethoate, dimpropyridaz, dinotefuran, diofenolan, emamectin, emamectin benzoate, endosulfan, esfenvalerate, ethiprole, etofenprox, epsilon-metofluthrin, etoxazole, fenbutatin oxide, fenitrothion, fenothiocarb, fenoxycarb, fenpropathrin, fenvalerate, fipronil, flometoquin (2-ethyl-3,7-dimethyl-6-[4-(trifluoromethoxy)phenoxy]-4-quinolinyl methyl carbonate), flonicamid, fluazaindolizine, flubendiamide, flucythrinate, flufenerim, flufenoxuron, flufenoxystrobin (methyl (αE)-2-[[2-chloro-4-(trifluoromethyl)phenoxy]methyl]-α-(methoxymethylene)benzeneacetate), fluensulfone (5-chloro-2-[(3,4,4-trifluoro-3-buten-1-yl)sulfonyl]thiazole), fluhexafon, fluopyram, flupiprole (1-[2,6-dichloro-4-(trifluoromethyl)phenyl]-5-[(2-methyl-2-propen-1-yl)amino]-4-[(trifluoromethyl)sulfinyl]-1H-pyrazole-3-carbonitrile), flupyradifurone (4-[[(6-chloro-3-pyridinyl)methyl](2,2-difluoroethyl)amino]-2(5H)-furanone), flupyrimin, fluvalinate, tau-fluvalinate, fluxametamide, fonophos, formetanate, fosthiazate, gamma-cyhalothrin, halofenozide, heptafluthrin ([2,3,5,6-tetrafluoro-4-(methoxymethyl)phenyl]methyl 2,2-dimethyl-3-[(1Z)-3,3,3-trifluoro-1-propen-1-yl]cyclopropanecarboxylate), hexaflumuron, hexythiazox, hydramethylnon, imidacloprid, indoxacarb, insecticidal soaps, isofenphos, isocycloseram, kappa-tefluthrin, lambda-cyhalothrin, lufenuron, malathion, meperfluthrin ([2,3,5,6-tetrafluoro-4-(methoxymethyl)phenyl]methyl (1R,3S)-3-(2,2-dichloroethenyl)-2,2-dimethylcyclopropanecarboxylate), metaflumizone, metaldehyde, methamidophos, methidathion, methiocarb, methomyl, methoprene, methoxychlor, metofluthrin, methoxyfenozide, epsilon-metofluthrin, epsilon-momfluorothrin, monocrotophos, monofluorothrin ([2,3,5,6-tetrafluoro-4-(methoxymethyl)phenyl]methyl 3-(2-cyano-1-propen-1-yl)-2,2-dimethylcyclopropanecarboxylate), nicofluprole, nicotine, nitenpyram, nithiazine, novaluron, noviflumuron, N-[1,1-dimethyl-2-(methylthio)ethyl]-7-fluoro-2-(3-pyridinyl)-2H-indazole-4-carboxamide, N-[1,1-dimethyl-2-(methylsulfinyl)ethyl]-7-fluoro-2-(3-pyridinyl)-2H-indazole-4-carboxamide, N-[1,1-dimethyl-2-(methylsulfonyl)ethyl]-7-fluoro-2-(3-pyridinyl)-2H-indazole-4-carboxamide, N-(1-methylcyclopropyl)-2-(3-pyridinyl)-2H-indazole-4-carboxamide and N-[1-(difluoromethyl)cyclopropyl]-2-(3-pyridinyl)-2H-indazole-4-carboxamide, oxamyl, oxazosulfyl, parathion, parathion-methyl, permethrin, phorate, phosalone, phosmet, phosphamidon, pirimicarb, profenofos, profluthrin, propargite, protrifenbute, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), pymetrozine, pyrafluprole, pyrethrin, pyridaben, pyridalyl, pyrifluquinazon, pyriminostrobin (methyl (αE)-2-[[[2-[(2,4-dichlorophenyl)amino]-6-(trifluoromethyl)-4-pyrimidinyl]oxy]methyl]-α-(methoxymethylene)benzeneacetate), pyriprole, pyriproxyfen, rotenone, ryanodine, silafluofen, spinetoram, spinosad, spirodiclofen, spiromesifen, spiropidion, spirotetramat, sulprofos, sulfoxaflor (N-[methyloxido[1-[6-(trifluoromethyl)-3-pyridinyl]ethyl]-λ⁴-sulfanylidene]cyanamide), tebufenozide, tebufenpyrad, teflubenzuron, tefluthrin, kappa-tefluthrin, terbufos, tetrachlorantraniliprole, tetrachlorvinphos, tetramethrin, tetramethylfluthrin ([2,3,5,6-tetrafluoro-4-(methoxymethyl)phenyl]methyl 2,2,3,3-tetramethylcyclopropanecarboxylate), tetraniliprole, thiacloprid, thiamethoxam, thiodicarb, thiosultap-sodium, tioxazafen (3-phenyl-5-(2-thienyl)-1,2,4-oxadiazole), tolfenpyrad, tralomethrin, triazamate, trichlorfon, triflumezopyrim (2,4-dioxo-1-(5-pyrimidinylmethyl)-3-[3-(trifluoromethyl)phenyl]-2H-pyrido[1,2-a]pyrimidinium inner salt), triflumuron, tyclopyrazoflor, zeta-cypermethrin, Bacillus thuringiensis delta-endotoxins, entomopathogenic bacteria, entomopathogenic viruses or entomopathogenic fungi.

Of note are insecticides such as abamectin, acetamiprid, acrinathrin, acynonapyr, afidopyropen, amitraz, avermectin, azadirachtin, benfuracarb, bensultap, bifenthrin, buprofezin, broflanilide, cadusafos, carbaryl, cartap, chlorantraniliprole, chloroprallethrin, chlorfenapyr, chlorpyrifos, clothianidin, cyantraniliprole, cyclaniliprole, cycloprothrin, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, epsilon-metofluthrin, esfenvalerate, ethiprole, etofenprox, etoxazole, fenitrothion, fenothiocarb, fenoxycarb, fenvalerate, fipronil, flometoquin, fluxametamide, flonicamid, flubendiamide, fluensulfone, flufenoxuron, flufenoxystrobin, flufensulfone, flupiprole, flupyrimin, flupyradifurone, fluvalinate, formetanate, fosthiazate, gamma-cyhalothrin, heptafluthrin, hexaflumuron, hydramethylnon, imidacloprid, indoxacarb, isocycloseram, kappa-tefluthrin, lambda-cyhalothrin, lufenuron, meperfluthrin, metaflumizone, methiodicarb, methomyl, methoprene, methoxyfenozide, metofluthrin, monofluorothrin, nitenpyram, nithiazine, novaluron, oxamyl, pyflubumide, pymetrozine, pyrethrin, pyridaben, pyridalyl, pyriminostrobin, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumezopyrim, triflumuron, tyclopyrazoflor, zeta-cypermethrin, Bacillus thuringiensis delta-endotoxins, all strains of Bacillus thuringiensis and all strains of nucleo polyhedrosis viruses.

One embodiment of biological agents for mixing with compounds of this disclosure include entomopathogenic bacteria such as Bacillus thuringiensis, and the encapsulated delta-endotoxins of Bacillus thuringiensis such as MVP® and MVPII® bioinsecticides prepared by the CellCap® process (CellCap®, MVP® and MVPII® are trademarks of Mycogen Corporation, Indianapolis, Ind., USA); entomopathogenic fungi such as green muscardine fungus; and entomopathogenic (both naturally occurring and genetically modified) viruses including baculovirus, nucleopolyhedro virus (NPV) such as Helicoverpa zea nucleopolyhedrovirus (HzNPV), Anagrapha falcifera nucleopolyhedrovirus (AfNPV); and granulosis virus (GV) such as Cydia pomonella granulosis virus (CpGV).

One embodiment of biological agents for mixing with compounds of this disclosure include one or a combination of (i) a bacterium of the genus Actinomycetes, Agrobacterium, Arthrobacter, Alcaligenes, Aureobacterium, Azobacter, Bacillus, Beijerinckia, Bradyrhizobium, Brevibacillus, Burkholderia, Chromobacterium, Clostridium, Clavibacter, Comamonas, Corynebacterium, Curtobacterium, Enterobacter, Flavobacterium, Gluconobacter, Hydrogenophaga, Klebsiella, Methylobacterium, Paenibacillus, Pasteuria, Photorhabdus, Phyllobacterium, Pseudomonas, Rhizobium, Serratia, Sphingobacterium, Stenotrophomonas, Streptomyces, Variovorax, or Xenorhabdus, for example a bacterium of Bacillus amyloliquefaciens, Bacillus cereus, Bacillus firmus, Bacillus, licheniformis, Bacillus pumilus, Bacillus sphaericus, Bacillus subtilis, Bacillus thuringiensis, Bradyrhizobium japonicum, Chromobacterium subtsugae, Pasteuria nishizawae, Pasteuria penetrans, Pasteuria usage, Pseudomonas fluorescens, and Streptomyces lydicus; (ii) a fungus such as green muscardine fungus; (iii) a virus including baculovirus, nucleopolyhedro virus such as Helicoverpa zea nucleopolyhedrovirus, Anagrapha falcifera nucleopolyhedrovirus; granulosis virus such as Cydia pomonella granulosis virus.

Of particular note is such a combination where the other invertebrate pest control active ingredient belongs to a different chemical class or has a different site of action than compounds of Formula 1. In certain instances, a combination with at least one other invertebrate pest control active ingredient having a similar spectrum of control but a different site of action will be particularly advantageous for resistance management. Thus, a composition of the present disclosure can further comprise a biologically effective amount of at least one additional invertebrate pest control active ingredient having a similar spectrum of control but belonging to a different chemical class or having a different site of action. These additional biologically active compounds or agents include, but are not limited to, acetylcholinesterase (AChE) inhibitors such as the carbamates methomyl, oxamyl, thiodicarb, triazamate, and the organophosphates chlorpyrifos; GABA-gated chloride channel antagonists such as the cyclodienes dieldrin and endosulfan, and the phenylpyrazoles ethiprole and fipronil; sodium channel modulators such as the pyrethroids bifenthrin, cyfluthrin, beta-cyfluthrin, cyhalothrin, lambda-cyhalothrin, cypermethrin, deltamethrin, dimefluthrin, esfenvalerate, metofluthrin and profluthrin; nicotinic acetylcholinereceptor (nAChR) agonists such as the neonicotinoids acetamiprid, clothianidin, dinotefuran, imidacloprid, nitenpyram, nithiazine, thiacloprid, and thiamethoxam, and sulfoxaflor; nicotinic acetylcholine receptor (nAChR) allosteric activators such as the spinosyns spinetoram and spinosad; chloride channel activators such as the avermectins abamectin and emamectin; juvenile hormone mimics such as diofenolan, methoprene, fenoxycarb and pyriproxyfen; selective homopteran feeding blockers such as pymetrozine and flonicamid; mite growth inhibitors such as etoxazole; inhibitors of mitochondrial ATP synthase such as propargite; ucouplers of oxidative phosphorylation via disruption of the proton gradient such as chlorfenapyr; nicotinic acetylcholine receptor (nAChR) channel blockers such as the nereistoxin analogs cartap; inhibitors of chitin biosynthesis such as the benzoylureas flufenoxuron, hexaflumuron, lufenuron, novaluron, noviflumuron and triflumuron, and buprofezin; dipteran moulting disrupters such as cyromazine; ecdysone receptor agonists such as the diacylhydrazines methoxyfenozide and tebufenozide; octopamine receptor agonists such as amitraz; mitochondrial complex III electron transport inhibitors such as hydramethylnon; mitochondrial complex I electron transport inhibitors such as pyridaben; voltage-dependent sodium channel blockers such as indoxacarb; inhibitors of acetyl CoA carboxylase such as the tetronic and tetramic acids spirodiclofen, spiromesifen and spirotetramat; mitochondrial complex II electron transport inhibitors such as the β-ketonitriles cyenopyrafen and cyflumetofen; ryanidine receptor modulators such as the anthranilic diamides chlorantraniliprole, cyantraniliprole and cyantraniliprole, diamides such as flubendiamide, and ryanodine receptor ligands such as ryanodine; compounds wherein the target site responsible for biological activity is unknown or uncharacterized such as azadirachtin, bifenazate, pyridalyl, pyrifluquinazon and triflumezopyrim; microbial disrupters of insect midgut membranes such as Bacillus thuringensis and the delta-endotoxins they produce and Bacillus sphaericus; and biological agents including nucleo polyhedro viruses (NPV) and other naturally occurring or genetically modified insecticidal viruses.

Further examples of biologically active compounds or agents with which compounds of this disclosure can be formulated are: fungicides such as acibenzolar-S-methyl, aldimorph, ametoctradin, aminopyrifen, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bitertanol, bixafen, blasticidin-S, boscalid, bromuconazole, bupirimate, buthiobate, carboxin, carpropamid, captafol, captan, carbendazim, chloroneb, chlorothalonil, chlozolinate, copper hydroxide, copper oxychloride, copper sulfate, coumoxystrobin, cyazofamid, cyflufenamid, cymoxanil, cyproconazole, cyprodinil, dichlobentiazox, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dipymetitrone, dithianon, dithiolanes, dodemorph, dodine, econazole, etaconazole, edifenphos, enoxastrobin (also known as enestroburin), epoxiconazole, ethaboxam, ethirimol, etridiazole, famoxadone, fenamidone, fenaminstrobin, fenarimol, fenbuconazole, fenfuram, fenhexamide, fenoxanil, fenpiclonil, fenpicoxamid, fenpropidin, fenpropimorph, fenpyrazamine, fentin acetate, fentin hydroxide, ferbam, ferimzone, flometoquin, florylpicoxamid, fluopimomide, fluazinam, fludioxonil, flufenoxystrobin, fluindapyr, flumorph, fluopicolide, fluopyram, flupentiofenox, fluoxapiprolin, fluoxastrobin, fluquinconazole, flusilazole, flusulfamide, flutianil, flutolanil, flutriafol, fluxapyroxad, folpet, fthalide (also known as phthalide), fuberidazole, furalaxyl, furametpyr, hexaconazole, hymexazole, guazatine, imazalil, imibenconazole, iminoctadine albesilate, iminoctadine triacetate, inpyrfluxam, iodicarb, ipconazole, ipfentrifluconazole, ipflufenoquin, isofetamid, iprobenfos, iprodione, iprovalicarb, isoflucypram, isoprothiolane, isopyrazam, isotianil, kasugamycin, kresoxim-methyl, lancotrione, mancozeb, mandipropamid, mandestrobin, maneb, mapanipyrin, mefentrifluconazole, mepronil, meptyldinocap, metalaxyl (including metalaxyl-M/mefenoxam), metconazole, methasulfocarb, metiram, metominostrobin, metyltetraprole, metrafenone, myclobutanil, naftitine, neo-asozin (ferric methanearsonate), nuarimol, octhilinone, ofurace, orysastrobin, oxadixyl, oxathiapiprolin, oxolinic acid, oxpoconazole, oxycarboxin, oxytetracycline, penconazole, pencycuron, penflufen, penthiopyrad, perfurazoate, phosphorous acid (including salts thereof, e.g., fosetyl-aluminm), picoxystrobin, piperalin, polyoxin, probenazole, prochloraz, procymidone, propamocarb, propiconazole, propineb, proquinazid, prothiocarb, prothioconazole, pydiflumetofen (Adepidyn®), pyraclostrobin, pyrametostrobin, pyrapropoyne, pyraoxystrobin, pyraziflumid, pyrazophos, pyribencarb, pyributacarb, pyridachlometyl, pyrifenox, pyriofenone, perisoxazole, pyrimethanil, pyrifenox, pyrrolnitrin, pyroquilon, quinconazole, quinmethionate, quinofumelin, quinoxyfen, quintozene, silthiofam, sedaxane, simeconazole, spiroxamine, streptomycin, sulfur, tebuconazole, tebufloquin, teclofthalam, tecloftalam, tecnazene, terbinafine, tetraconazole, thiabendazole, thifluzamide, thiophanate, thiophanate-methyl, thiram, tiadinil, tolclofos-methyl, tolprocarb, tolyfluanid, triadimefon, triadimenol, triarimol, triazoxide, tribasic copper sulfate, triclopyricarb, tridemorph, trifloxystrobin, triflumizole, trimoprhamide tricyclazole, trifloxystrobin, triforine, triticonazole, uniconazole, validamycin, valifenalate (also known as valifenal), vinclozolin, zineb, ziram, zoxamide and 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone; nematocides such as fluopyram, spirotetramat, thiodicarb, fosthiazate, abamectin, iprodione, fluensulfone, dimethyl disulfide, tioxazafen, 1,3-dichloropropene (1,3-D), metam (sodium and potassium), dazomet, chloropicrin, fenamiphos, ethoprophos, cadusaphos, terbufos, imicyafos, oxamyl, carbofuran, tioxazafen, Bacillus firmus and Pasteuria nishizawae; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad.

In certain instances, combinations of a compound of this disclosure with other biologically active (particularly invertebrate pest control) compounds or agents (i.e. active ingredients) can result in an enhanced effect. Reducing the quantity of active ingredients released in the environment while ensuring effective pest control is always desirable. When enhanced invertebrate pest control occurs at application rates giving agronomically satisfactory levels of invertebrate pest control, such combinations can be advantageous for reducing crop production cost and decreasing environmental load.

Compounds of this disclosure and compositions thereof can be applied to plants genetically transformed to express proteins toxic to invertebrate pests (such as Bacillus thuringiensis delta-endotoxins). Such an application may provide a broader spectrum of plant protection and be advantageous for resistance management. The exogenously applied invertebrate pest control compounds of this disclosure in combination with the expressed toxin proteins may provide an enhanced effect.

General references for these agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U. K., 2003 and The BioPesticide Manual, 2^{nd} Edition, L. G. Copping, Ed., British Crop Protection Council, Farnham, Surrey, U. K., 2001.

Compounds of this disclosure can be combined or formulated with polynucleotides including, but not limited to, DNA, RNA, and/or chemically modified nucleotides influencing the amount of a particular target through down regulation, interference, suppression or silencing of the genetically derived transcript that render an insecticidal effect.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components can expand the spectrum of invertebrate pests controlled beyond the spectrum controlled by the compound of Formula 1 alone.

Table A lists specific combinations of a compound of Formula 1 or Formula 1′ with other invertebrate pest control agents illustrative of the mixtures, compositions and methods of the present disclosure. The first column of Table A lists the specific invertebrate pest control agents (e.g., “Abamectin” in the first line). The second column of Table A lists the mode of action (if known) or chemical class of the invertebrate pest control agents. The third column of Table A lists embodiment(s) of ranges of weight ratios for rates at which the invertebrate pest control agent can be applied relative to a compound of Formula 1 or Formula 1′ (e.g., “50:1 to 1:50” of abamectin relative to a compound of Formula 1 or Formula 1′ by weight). Thus, for example, the first line of Table A specifically discloses the combination of a compound of Formula 1 or Formula 1′ with abamectin can be applied in a weight ratio between 50:1 to 1:50. The remaining lines of Table A are to be construed similarly. Of further note Table A lists specific combinations of a compound of Formula 1 or Formula 1′ with other invertebrate pest control agents illustrative of the mixtures, compositions and methods of the present disclosure and includes additional embodiments of weight ratio ranges for application rates.

TABLE A

| Invertebrate Pest | Mode of Action or | Typical |
|---|---|---|
| Control Agent | Chemical Class | Weight Ratio |
| Abamectin | chloride channel activator | 50:1 to 1:50 |
| Acetamiprid | nicotinic acetylcholinereceptor | 150:1 to 1:200 |
| | (nAChR) agonist | |
| Amitraz | octopamine receptor agonists | 200:1 to 1:100 |
| Avermectin | macrocyclic lactones | 50:1 to 1:50 |
| Azadirachtin | unknown site of action | 100:1 to 1:120 |
| Beta-cyfluthrin | sodium channel modulators | 150:1 to 1:200 |
| Bifenthrin | sodium channel modulators | 100:1 to 1:10 |
| Buprofezin | chitin biosynthesis inhibitors | 500:1 to 1:50 |
| Cartap | nicotinic acetylcholine receptor | 100:1 to 1:200 |
| | (nAChR) channel blocker | |
| Chlorantraniliprole | ryanodine receptor modulator | 100:1 to 1:120 |
| Chlorfenapyr | uncouplers of oxidative | 300:1 to 1:200 |
| | phosphorylation | |
| Chlorpyrifos | acetylcholinesterase inhibitor | 500:1 to 1:200 |
| Clothianidin | nicotinic acetylcholine receptor | 100:1 to 1:400 |
| | (nAChR) agonist | |
| Cyantraniliprole | Ryanodine receptor modulator | 100:1 to 1:120 |
| Cyfluthrin | sodium channel modulator | 150:1 to 1:200 |
| Cyhalothrin | sodium channel modulator | 150:1 to 1:200 |
| Cypermethrin | sodium channel modulator | 150:1 to 1:200 |
| Cyromazine | dipteran moulting disrupter | 400:1 to 1:50 |
| Deltamethrin | sodium channel modulators | 50:1 to 1:400 |
| Dieldrin | GABA-gated chloride | 200:1 to 1:100 |
| | channel antagonist | |
| Dinotefuran | nicotinic acetylcholine receptor | 150:1 to 1:200 |
| | (nAChR) agonist | |
| Diofenolan | juvenile hormone mimic | 150:1 to 1:200 |
| Emamectin | chloride channel activator | 50:1 to 1:10 |
| Endosulfan | GABA-gated chloride | 200:1 to 1:100 |
| | channel antagonist | |
| Esfenvalerate | sodium channel modulator | 100:1 to 1:400 |
| Ethiprole | GABA-regulated chloride | 200:1 to 1:100 |
| | channel antagonist | |
| Fenothiocarb | | 150:1 to 1:200 |
| Fenoxycarb | juvenile hormone mimic | 500:1 to 1:100 |
| Fenvalerate | sodium channel modulator | 150:1 to 1:200 |
| Fipronil | GABA-regulated chloride | 150:1 to 1:100 |
| | channel antagonist | |
| Flonicamid | selective homopteran feeding | 200:1 to 1:100 |
| | blocker | |
| Flubendiamide | ryanodine receptor modulator | 100:1 to 1:120 |
| Flufenoxuron | chitin biosynthesis inhibitor | 200:1 to 1:100 |
| Hexaflumuron | chitin biosynthesis inhibitor | 300:1 to 1:50 |
| Hydramethylnon | mitochondrial Complex III | 150:1 to 1:250 |
| | electron transport inhibitors | |
| Imidacloprid | nicotinic acetylcholine receptor | 1000:1 to 1:1000 |
| | (nAChR) agonist | |
| Indoxacarb | voltage-dependent sodium | 200:1 to 1:50 |
| | channel blocker | |
| Lambda-cyhalothrin | sodium channel modulator | 50:1 to 1:250 |
| Lufenuron | chitin biosynthesis inhibitor | 500:1 to 1:250 |
| Metaflumizone | voltage-dependent sodium | 200:1 to 1:200 |
| | channel blocker | |
| Methomyl | acetylcholinesterase inhibitor | 500:1 to 1:100 |
| Methoprene | juvenile hormone mimic | 500:1 to 1:100 |
| Methoxyfenozide | ecdysone receptor agonist | 50:1 to 1:50 |
| Nitenpyram | nicotinic acetylcholine receptor | 150:1 to 1:200 |
| | (nAChR) agonist | |
| Nithiazine | nicotinic acetylcholine receptor | 150:1 to 1:200 |
| | (nAChR) agonist | |
| Novaluron | chitin biosynthesis inhibitor | 500:1 to 1:150 |
| Oxamyl | acetylcholinesterase inhibitors | 200:1 to 1:200 |
| Pymetrozine | selective homopteran feeding | 200:1 to 1:100 |
| | blocker | |
| Pyrethrin | sodium channel modulator | 100:1 to 1:10 |
| Pyridaben | mitochondrial Complex I | 200:1 to 1:100 |
| | electron transport inhibitor | |
| Pyridalyl | unknown site of action | 200:1 to 1:100 |
| Pyriproxyfen | juvenile hormone mimic | 500:1 to 1:100 |
| Ryanodine | ryanodine receptor ligand | 100:1 to 1:120 |
| Spinetoram | nicotinic acetylcholine receptor | 150:1 to 1:100 |
| | (nAChR) allosteric activator | |
| Spinosad | nicotinic acetylcholine receptor | 500:1 to 1:10 |
| | (nAChR) allosteric activators | |
| Spirodiclofen | acetyl CoA carboxylase inhibitor | 200:1 to 1:200 |
| Spiromesifen | acetyl CoA carboxylase inhibitor | 200:1 to 1:200 |
| Tebufenozide | ecdysone receptor agonist | 500:1 to 1:250 |
| Thiacloprid | nicotinic acetylcholine receptor | 100:1 to 1:200 |
| | (nAChR) agonist | |
| Thiamethoxam | nicotinic acetylcholine receptor | 1250:1 to 1:1000 |
| | (nAChR) agonist | |
| Thiodicarb | acetylcholinesterase inhibitors | 500:1 to 1:400 |
| Thiosultap-sodium | Nicotinic acetylcholine receptor | 150:1 to 1:100 |
| | (nAChR) channel blocker | |
| Tralomethrin | sodium channel modulator | 150:1 to 1:200 |
| Triazamate | acetyl cholinesterase inhibitors | 250:1 to 1:100 |
| Triflumezopyrim | | |
| Triflumuron | chitin synthesis inhibitor | 200:1 to 1:100 |
| Bacillus | biological agents | 50:1 to 1:10 |
| thuringiensis | | |
| Bacillus | biological agents | 50:1 to 1:10 |
| thuringiensis | | |
| delta-endotoxin | | |
| NPV (e.g., Gemstar) | biological agents | 50:1 to 1:10 |

Of note is the composition of the present disclosure wherein the at least one additional biologically active compound or agent is selected from the Invertebrate Pest Control Agents listed in Table A above.

The weight ratios of a compound, including a compound of Formula 1 or Formula 1′, an N-oxide or a salt thereof, to the additional invertebrate pest control agent typically are between 1000:1 and 1:1000, with one embodiment being between 500:1 and 1:500, another embodiment being between 250:1 and 1:200 and another embodiment being between 100:1 and 1:50.

Listed below in Table B1 are embodiments of specific compositions comprising a compound of Formula 1 (compound numbers (Cmpd. No.) refer to compounds in Index Table A) and an additional invertebrate pest control agent.

TABLE B1

| Mixture | Cmpd. | | Invertebrate Pest |
|---|---|---|---|
| No. | No. | and | Control Agent |
| B1-1 | 1 | and | Abamectin |
| B1-2 | 1 | and | Acetamiprid |
| B1-3 | 1 | and | Amitraz |
| B1-4 | 1 | and | Avermectin |
| B1-5 | 1 | and | Azadirachtin |
| B1-6 | 1 | and | Bensultap |
| B1-7 | 1 | and | Beta-cyfluthrin |
| B1-8 | 1 | and | Bifenthrin |
| B1-9 | 1 | and | Buprofezin |
| B1-10 | 1 | and | Cartap |
| B1-11 | 1 | and | Chlorantraniliprole |
| B1-12 | 1 | and | Chlorfenapyr |
| B1-13 | 1 | and | Chlorpyrifos |
| B1-14 | 1 | and | Clothianidin |
| B1-15 | 1 | and | Cyantraniliprole |
| B1-16 | 1 | and | Cyfluthrin |
| B1-17 | 1 | and | Cyhalothrin |
| B1-18 | 1 | and | Cypermethrin |
| B1-19 | 1 | and | Cyromazine |
| B1-20 | 1 | and | Deltamethrin |
| B1-21 | 1 | and | Dieldrin |
| B1-22 | 1 | and | Dinotefuran |
| B1-23 | 1 | and | Diofenolan |
| B1-24 | 1 | and | Emamectin |
| B1-25 | 1 | and | Endosulfan |
| B1-26 | 1 | and | Esfenvalerate |
| B1-27 | 1 | and | Ethiprole |
| B1-28 | 1 | and | Fenothiocarb |
| B1-29 | 1 | and | Fenoxycarb |
| B1-30 | 1 | and | Fenvalerate |
| B1-31 | 1 | and | Fipronil |
| B1-32 | 1 | and | Flonicamid |
| B1-33 | 1 | and | Flubendiamide |
| B1-34 | 1 | and | Flufenoxuron |
| B1-35 | 1 | and | Hexaflumuron |
| B1-36 | 1 | and | Hydramethylnon |
| B1-37 | 1 | and | Imidacloprid |
| B1-38 | 1 | and | Indoxacarb |
| B1-39 | 1 | and | Lambda-cyhalothrin |
| B1-40 | 1 | and | Lufenuron |
| B1-41 | 1 | and | Metaflumizone |
| B1-42 | 1 | and | Methomyl |
| B1-43 | 1 | and | Methoprene |
| B1-44 | 1 | and | Methoxyfenozide |
| B1-45 | 1 | and | Nitenpyram |
| B1-46 | 1 | and | Nithiazine |
| B1-47 | 1 | and | Novaluron |
| B1-48 | 1 | and | Oxamyl |
| B1-49 | 1 | and | Phosmet |
| B1-50 | 1 | and | Pymetrozine |
| B1-51 | 1 | and | Pyrethrin |
| B1-52 | 1 | and | Pyridaben |
| B1-53 | 1 | and | Pyridalyl |
| B1-54 | 1 | and | Pyriproxyfen |
| B1-55 | 1 | and | Ryanodine |
| B1-56 | 1 | and | Spinetoram |
| B1-57 | 1 | and | Spinosad |
| B1-58 | 1 | and | Spirodiclofen |
| B1-59 | 1 | and | Spiromesifen |
| B1-60 | 1 | and | Spirotetramat |
| B1-61 | 1 | and | Sulfoxaflor |
| B1-62 | 1 | and | Tebufenozide |
| B1-63 | 1 | and | Tefluthrin |
| B1-64 | 1 | and | Thiacloprid |
| B1-65 | 1 | and | Thiamethoxam |
| B1-66 | 1 | and | Thiodicarb |
| B1-67 | 1 | and | Thiosultap-sodium |
| B1-68 | 1 | and | Tolfenpyrad |
| B1-69 | 1 | and | Tralomethrin |
| B1-70 | 1 | and | Triazamate |
| B1-71 | 1 | and | Triflumezopyrim |
| B1-72 | 1 | and | Triflumuron |
| B1-73 | 1 | and | Bacillus thuringiensis |
| B1-74 | 1 | and | Bacillus thuringiensis delta-endotoxin |
| B1-75 | 1 | and | NPV (e.g., Gemstar) |

The specific mixtures listed in Table B1 typically combine a compound of Formula 1 with the other invertebrate pest agent in the ratios specified in Table A.

Listed below in Table C1 are specific mixtures comprising a compound of Formula 1 (compound numbers (Cmpd. No.) refer to compounds in Index Table A) and an additional invertebrate pest control agent. Table C1 further list specific weight ratios typical of the mixtures of Table Cl. For example, the first weight ratio entry of the first line of Table C1 specifically discloses the mixture of Compound 1 of Index Table A with abamectin applied in a weight ratio of 100 parts Compound 1 to 1 part abamectin.

TABLE C1

| Mixture | Cmpd. | | Invertebrate Pest | | | | | | | | | |
|---|---|---|---|---|---|---|---|---|---|---|---|---|
| No. | No. | and | Control Agent | Typical Mixture Ratios (by weight) | | | | | | | | |
| C1-1 | 1 | and | Abamectin | 100:1 | 10:1 | 5:1 | 2:1 | 1:1 | 1:2 | 1:5 | 1:10 | 1:100 |
| C1-2 | 1 | and | Acetamiprid | 100:1 | 10:1 | 5:1 | 2:1 | 1:1 | 1:2 | 1:5 | 1:10 | 1:100 |
| C1-3 | 1 | and | Amitraz | 100:1 | 10:1 | 5:1 | 2:1 | 1:1 | 1:2 | 1:5 | 1:10 | 1:100 |
| C1-4 | 1 | and | Avermectin | 100:1 | 10:1 | 5:1 | 2:1 | 1:1 | 1:2 | 1:5 | 1:10 | 1:100 |
| C1-5 | 1 | and | Azadirachtin | 100:1 | 10:1 | 5:1 | 2:1 | 1:1 | 1:2 | 1:5 | 1:10 | 1:100 |
| C1-6 | 1 | and | Bensultap | 100:1 | 10:1 | 5:1 | 2:1 | 1:1 | 1:2 | 1:5 | 1:10 | 1:100 |
| C1-7 | 1 | and | Beta-cyfluthrin | 100:1 | 10:1 | 5:1 | 2:1 | 1:1 | 1:2 | 1:5 | 1:10 | 1:100 |
| C1-8 | 1 | and | Bifenthrin | 100:1 | 10:1 | 5:1 | 2:1 | 1:1 | 1:2 | 1:5 | 1:10 | 1:100 |
| C1-9 | 1 | and | Buprofezin | 100:1 | 10:1 | 5:1 | 2:1 | 1:1 | 1:2 | 1:5 | 1:10 | 1:100 |
| C1-10 | 1 | and | Cartap | 100:1 | 10:1 | 5:1 | 2:1 | 1:1 | 1:2 | 1:5 | 1:10 | 1:100 |
| C1-11 | 1 | and | Chlorantraniliprole | 100:1 | 10:1 | 5:1 | 2:1 | 1:1 | 1:2 | 1:5 | 1:10 | 1:100 |
| C1-12 | 1 | and | Chlorfenapyr | 100:1 | 10:1 | 5:1 | 2:1 | 1:1 | 1:2 | 1:5 | 1:10 | 1:100 |
| C1-13 | 1 | and | Chlorpyrifos | 100:1 | 10:1 | 5:1 | 2:1 | 1:1 | 1:2 | 1:5 | 1:10 | 1:100 |
| C1-14 | 1 | and | Clothianidin | 100:1 | 10:1 | 5:1 | 2:1 | 1:1 | 1:2 | 1:5 | 1:10 | 1:100 |
| C1-15 | 1 | and | Cyantraniliprole | 100:1 | 10:1 | 5:1 | 2:1 | 1:1 | 1:2 | 1:5 | 1:10 | 1:100 |
| C1-16 | 1 | and | Cyfluthrin | 100:1 | 10:1 | 5:1 | 2:1 | 1:1 | 1:2 | 1:5 | 1:10 | 1:100 |
| C1-17 | 1 | and | Cyhalothrin | 100:1 | 10:1 | 5:1 | 2:1 | 1:1 | 1:2 | 1:5 | 1:10 | 1:100 |
| C1-18 | 1 | and | Cypermethrin | 100:1 | 10:1 | 5:1 | 2:1 | 1:1 | 1:2 | 1:5 | 1:10 | 1:100 |
| C1-19 | 1 | and | Cyromazine | 100:1 | 10:1 | 5:1 | 2:1 | 1:1 | 1:2 | 1:5 | 1:10 | 1:100 |
| C1-20 | 1 | and | Deltamethrin | 100:1 | 10:1 | 5:1 | 2:1 | 1:1 | 1:2 | 1:5 | 1:10 | 1:100 |
| C1-21 | 1 | and | Dieldrin | 100:1 | 10:1 | 5:1 | 2:1 | 1:1 | 1:2 | 1:5 | 1:10 | 1:100 |
| C1-22 | 1 | and | Dinotefuran | 100:1 | 10:1 | 5:1 | 2:1 | 1:1 | 1:2 | 1:5 | 1:10 | 1:100 |
| C1-23 | 1 | and | Diofenolan | 100:1 | 10:1 | 5:1 | 2:1 | 1:1 | 1:2 | 1:5 | 1:10 | 1:100 |
| C1-24 | 1 | and | Emamectin | 100:1 | 10:1 | 5:1 | 2:1 | 1:1 | 1:2 | 1:5 | 1:10 | 1:100 |
| C1-25 | 1 | and | Endosulfan | 100:1 | 10:1 | 5:1 | 2:1 | 1:1 | 1:2 | 1:5 | 1:10 | 1:100 |
| C1-26 | 1 | and | Esfenvalerate | 100:1 | 10:1 | 5:1 | 2:1 | 1:1 | 1:2 | 1:5 | 1:10 | 1:100 |
| C1-27 | 1 | and | Ethiprole | 100:1 | 10:1 | 5:1 | 2:1 | 1:1 | 1:2 | 1:5 | 1:10 | 1:100 |
| C1-28 | 1 | and | Fenothiocarb | 100:1 | 10:1 | 5:1 | 2:1 | 1:1 | 1:2 | 1:5 | 1:10 | 1:100 |
| C1-29 | 1 | and | Fenoxycarb | 100:1 | 10:1 | 5:1 | 2:1 | 1:1 | 1:2 | 1:5 | 1:10 | 1:100 |
| C1-30 | 1 | and | Fenvalerate | 100:1 | 10:1 | 5:1 | 2:1 | 1:1 | 1:2 | 1:5 | 1:10 | 1:100 |
| C1-31 | 1 | and | Fipronil | 100:1 | 10:1 | 5:1 | 2:1 | 1:1 | 1:2 | 1:5 | 1:10 | 1:100 |
| C1-32 | 1 | and | Flonicamid | 100:1 | 10:1 | 5:1 | 2:1 | 1:1 | 1:2 | 1:5 | 1:10 | 1:100 |
| C1-33 | 1 | and | Flubendiamide | 100:1 | 10:1 | 5:1 | 2:1 | 1:1 | 1:2 | 1:5 | 1:10 | 1:100 |
| C1-34 | 1 | and | Flufenoxuron | 100:1 | 10:1 | 5:1 | 2:1 | 1:1 | 1:2 | 1:5 | 1:10 | 1:100 |
| C1-35 | 1 | and | Hexaflumuron | 100:1 | 10:1 | 5:1 | 2:1 | 1:1 | 1:2 | 1:5 | 1:10 | 1:100 |
| C1-36 | 1 | and | Hydramethylnon | 100:1 | 10:1 | 5:1 | 2:1 | 1:1 | 1:2 | 1:5 | 1:10 | 1:100 |
| C1-37 | 1 | and | Imidacloprid | 100:1 | 10:1 | 5:1 | 2:1 | 1:1 | 1:2 | 1:5 | 1:10 | 1:100 |
| C1-38 | 1 | and | Indoxacarb | 100:1 | 10:1 | 5:1 | 2:1 | 1:1 | 1:2 | 1:5 | 1:10 | 1:100 |
| C1-39 | 1 | and | Lambda- | 100:1 | 10:1 | 5:1 | 2:1 | 1:1 | 1:2 | 1:5 | 1:10 | 1:100 |
| | | | cyhalothrin | | | | | | | | | |
| C1-40 | 1 | and | Lufenuron | 100:1 | 10:1 | 5:1 | 2:1 | 1:1 | 1:2 | 1:5 | 1:10 | 1:100 |
| C1-41 | 1 | and | Metaflumizone | 100:1 | 10:1 | 5:1 | 2:1 | 1:1 | 1:2 | 1:5 | 1:10 | 1:100 |
| C1-42 | 1 | and | Methomyl | 100:1 | 10:1 | 5:1 | 2:1 | 1:1 | 1:2 | 1:5 | 1:10 | 1:100 |
| C1-43 | 1 | and | Methoprene | 100:1 | 10:1 | 5:1 | 2:1 | 1:1 | 1:2 | 1:5 | 1:10 | 1:100 |
| C1-44 | 1 | and | Methoxyfenozide | 100:1 | 10:1 | 5:1 | 2:1 | 1:1 | 1:2 | 1:5 | 1:10 | 1:100 |
| C1-45 | 1 | and | Nitenpyram | 100:1 | 10:1 | 5:1 | 2:1 | 1:1 | 1:2 | 1:5 | 1:10 | 1:100 |
| C1-46 | 1 | and | Nithiazine | 100:1 | 10:1 | 5:1 | 2:1 | 1:1 | 1:2 | 1:5 | 1:10 | 1:100 |
| C1-47 | 1 | and | Novaluron | 100:1 | 10:1 | 5:1 | 2:1 | 1:1 | 1:2 | 1:5 | 1:10 | 1:100 |
| C1-48 | 1 | and | Oxamyl | 100:1 | 10:1 | 5:1 | 2:1 | 1:1 | 1:2 | 1:5 | 1:10 | 1:100 |
| C1-49 | 1 | and | Phosmet | 100:1 | 10:1 | 5:1 | 2:1 | 1:1 | 1:2 | 1:5 | 1:10 | 1:100 |
| C1-50 | 1 | and | Pymetrozine | 100:1 | 10:1 | 5:1 | 2:1 | 1:1 | 1:2 | 1:5 | 1:10 | 1:100 |
| C1-51 | 1 | and | Pyrethrin | 100:1 | 10:1 | 5:1 | 2:1 | 1:1 | 1:2 | 1:5 | 1:10 | 1:100 |
| C1-52 | 1 | and | Pyridaben | 100:1 | 10:1 | 5:1 | 2:1 | 1:1 | 1:2 | 1:5 | 1:10 | 1:100 |
| C1-53 | 1 | and | Pyridalyl | 100:1 | 10:1 | 5:1 | 2:1 | 1:1 | 1:2 | 1:5 | 1:10 | 1:100 |
| C1-54 | 1 | and | Pyriproxyfen | 100:1 | 10:1 | 5:1 | 2:1 | 1:1 | 1:2 | 1:5 | 1:10 | 1:100 |
| C1-55 | 1 | and | Ryanodine | 100:1 | 10:1 | 5:1 | 2:1 | 1:1 | 1:2 | 1:5 | 1:10 | 1:100 |
| C1-56 | 1 | and | Spinetoram | 100:1 | 10:1 | 5:1 | 2:1 | 1:1 | 1:2 | 1:5 | 1:10 | 1:100 |
| C1-57 | 1 | and | Spinosad | 100:1 | 10:1 | 5:1 | 2:1 | 1:1 | 1:2 | 1:5 | 1:10 | 1:100 |
| C1-58 | 1 | and | Spirodiclofen | 100:1 | 10:1 | 5:1 | 2:1 | 1:1 | 1:2 | 1:5 | 1:10 | 1:100 |
| C1-59 | 1 | and | Spiromesifen | 100:1 | 10:1 | 5:1 | 2:1 | 1:1 | 1:2 | 1:5 | 1:10 | 1:100 |
| C1-60 | 1 | and | Spirotetramat | 100:1 | 10:1 | 5:1 | 2:1 | 1:1 | 1:2 | 1:5 | 1:10 | 1:100 |
| C1-61 | 1 | and | Sulfoxaflor | 100:1 | 10:1 | 5:1 | 2:1 | 1:1 | 1:2 | 1:5 | 1:10 | 1:100 |
| C1-62 | 1 | and | Tebufenozide | 100:1 | 10:1 | 5:1 | 2:1 | 1:1 | 1:2 | 1:5 | 1:10 | 1:100 |
| C1-63 | 1 | and | Tefluthrin | 100:1 | 10:1 | 5:1 | 2:1 | 1:1 | 1:2 | 1:5 | 1:10 | 1:100 |
| C1-64 | 1 | and | Thiacloprid | 100:1 | 10:1 | 5:1 | 2:1 | 1:1 | 1:2 | 1:5 | 1:10 | 1:100 |
| C1-65 | 1 | and | Thiamethoxam | 100:1 | 10:1 | 5:1 | 2:1 | 1:1 | 1:2 | 1:5 | 1:10 | 1:100 |
| C1-66 | 1 | and | Thiodicarb | 100:1 | 10:1 | 5:1 | 2:1 | 1:1 | 1:2 | 1:5 | 1:10 | 1:100 |
| C1-67 | 1 | and | Thiosultap-sodium | 100:1 | 10:1 | 5:1 | 2:1 | 1:1 | 1:2 | 1:5 | 1:10 | 1:100 |
| C1-68 | 1 | and | Tolfenpyrad | 100:1 | 10:1 | 5:1 | 2:1 | 1:1 | 1:2 | 1:5 | 1:10 | 1:100 |
| C1-69 | 1 | and | Tralomethrin | 100:1 | 10:1 | 5:1 | 2:1 | 1:1 | 1:2 | 1:5 | 1:10 | 1:100 |
| C1-70 | 1 | and | Triazamate | 100:1 | 10:1 | 5:1 | 2:1 | 1:1 | 1:2 | 1:5 | 1:10 | 1:100 |
| C1-71 | 1 | and | Triflumezopyrim | 100:1 | 10:1 | 5:1 | 2:1 | 1:1 | 1:2 | 1:5 | 1:10 | 1:100 |
| C1-72 | 1 | and | Triflumuron | 100:1 | 10:1 | 5:1 | 2:1 | 1:1 | 1:2 | 1:5 | 1:10 | 1:100 |
| C1-73 | 1 | and | Bacillus | 100:1 | 10:1 | 5:1 | 2:1 | 1:1 | 1:2 | 1:5 | 1:10 | 1:100 |
| | | | thuringiensis | | | | | | | | | |
| C1-74 | 1 | and | Bacillus | 100:1 | 10:1 | 5:1 | 2:1 | 1:1 | 1:2 | 1:5 | 1:10 | 1:100 |
| | | | thuringiensis | | | | | | | | | |
| | | | delta-endotoxin | | | | | | | | | |
| C1-75 | 1 | and | NPV (e.g., | 100:1 | 10:1 | 5:1 | 2:1 | 1:1 | 1:2 | 1:5 | 1:10 | 1:100 |
| | | | Gemstar) | | | | | | | | | |

Listed below in Table Dl are embodiments of specific compositions comprising a compound of Formula 1 (compound numbers (Cmpd. No.) refer to compounds in Index Table A) and an additional fungicide.

TABLE D1

| Mixture | Cmpd. | | |
|---|---|---|---|
| No. | No. | and | Fungicide |
| D1-1 | 1 | and | Probenazole |
| D1-2 | 1 | and | Tiadinil |
| D1-3 | 1 | and | Isotianil |
| D1-4 | 1 | and | Pyroquilon |
| D1-5 | 1 | and | Metominostrobin |
| D1-6 | 1 | and | Flutolanil |
| D1-7 | 1 | and | Validamycin |
| D1-8 | 1 | and | Furametpyr |
| D1-9 | 1 | and | Pencycuron |
| D1-10 | 1 | and | Simeconazole |
| D1-11 | 1 | and | Orysastrobin |
| D1-12 | 1 | and | Trifloxystrobin |
| D1-13 | 1 | and | Isoprothiolane |
| D1-14 | 1 | and | Azoxystrobin |
| D1-15 | 1 | and | Tricyclazole |
| D1-16 | 1 | and | Hexaconazole |
| D1-17 | 1 | and | Difenoconazole |
| D1-18 | 1 | and | Cyproconazole |
| D1-19 | 1 | and | Propiconazole |
| D1-20 | 1 | and | Fenoxanil |
| D1-21 | 1 | and | Ferimzone |
| D1-22 | 1 | and | Fthalide |
| D1-23 | 1 | and | Kasugamycin |
| D1-24 | 1 | and | Picoxystrobin |
| D1-25 | 1 | and | Penthiopyrad |
| D1-26 | 1 | and | Famoxadone |
| D1-27 | 1 | and | Cymoxanil |
| D1-28 | 1 | and | Proquinazid |
| D1-29 | 1 | and | Flusilazole |
| D1-30 | 1 | and | Mancozeb |
| D1-31 | 1 | and | Copper hydroxide |
| D1-32 | 1 | and | oxathiapiprolin |

Invertebrate pests are controlled in agronomic and nonagronomic applications by applying one or more compounds of this disclosure, typically in the form of a composition, in a biologically effective amount, to the environment of the pests, including the agronomic and/or nonagronomic locus of infestation, to the area to be protected, or directly on the pests to be controlled.

Thus the present disclosure comprises a method for controlling an invertebrate pest in agronomic and/or nonagronomic applications, comprising contacting the invertebrate pest or its environment with a biologically effective amount of one or more of the compounds of the disclosure, or with a composition comprising at least one such compound or a composition comprising at least one such compound and a biologically effective amount of at least one additional biologically active compound or agent. Examples of suitable compositions comprising a compound of the disclosure and a biologically effective amount of at least one additional biologically active compound or agent include granular compositions wherein the additional active compound is present on the same granule as the compound of the disclosure or on granules separate from those of the compound of the disclosure.

To achieve contact with a compound or composition of the disclosure to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a compound of the disclosure can be applied to the plant foliage or the soil. Compounds of this disclosure can also be effectively delivered through plant uptake by contacting the plant with a composition comprising a compound of this disclosure applied as a soil drench of a liquid formulation, a granular formulation to the soil, a nursery box treatment or a dip of transplants. Of note is a composition of the present disclosure in the form of a soil drench liquid formulation. Also of note is a method for controlling an invertebrate pest comprising contacting the invertebrate pest or its environment with a biologically effective amount of a compound of the present disclosure or with a composition comprising a biologically effective amount of a compound of the present disclosure. Of further note is this method wherein the environment is soil and the composition are applied to the soil as a soil drench formulation. Of further note is that compounds of this disclosure are also effective by localized application to the locus of infestation. Other methods of contact include application of a compound or a composition of the disclosure by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the disclosure. The compounds of this disclosure can also be impregnated into materials for fabricating invertebrate control devices (e.g., insect netting).

Compounds of the disclosure are useful in treating all plants, plant parts and seeds. Plant and seed varieties and cultivars can be obtained by conventional propagation and breeding methods or by genetic engineering methods. Genetically modified plants or seeds (transgenic plants or seeds) are those in which a heterologous gene (transgene) has been stably integrated into the plant's or seed's genome. A transgene that is defined by its particular location in the plant genome is called a transformation or transgenic event.

Genetically modified plant and seed cultivars which can be treated according to the disclosure include those that are resistant against one or more biotic stresses (pests such as nematodes, insects, mites, fungi, etc.) or abiotic stresses (drought, cold temperature, soil salinity, etc.), or that contain other desirable characteristics. Plants and seeds can be genetically modified to exhibit traits of, for example, herbicide tolerance, insect-resistance, modified oil profiles or drought tolerance. Useful genetically modified plants and seeds containing single gene transformation events or combinations of transformation events are listed in Table Z. Additional information for the genetic modifications listed in Table Z can be obtained from the following databases:

http://www2.oecd.org/biotech/byidentifier.aspx

http://www.aphis.usda.go

http://gmoinfo.jrc.ec.europa.eu

The following abbreviations are used in Table Z which follows: tol. is tolerance, res. is resistance, SU is sulfonylurea, ALS is acetolactate synthase, HPPD is 4-Hydroxyphenylpyruvate Dioxygenase, NA is Not Available?

TABLE Z

| Crop | Event Name | Event Code | Trait(s) | Gene(s) |
|---|---|---|---|---|
| Alfalfa | J101 | MON-00101-8 | Glyphosate tol. | cp4 epsps (aroA:CP4) |
| Alfalfa | J163 | MON-ØØ163-7 | Glyphosate tol. | cp4 epsps (aroA:CP4) |
| Canola* | 23-18-17 (Event | CGN-89465-2 | High lauric acid oil | te |
| | 18) | | | |
| Canola* | 23-198 (Event 23) | CGN-89465-2 | High lauric acid oil | te |
| Canola* | 61061 | DP-Ø61Ø61-7 | Glyphosate tol. | gat4621 |
| Canola* | 73496 | DP-Ø73496-4 | Glyphosate tol. | gat4621 |
| Canola* | GT200 (RT200) | MON-89249-2 | Glyphosate tol. | cp4 epsps (aroA:CP4); |
| | | | | goxv247 |
| Canola* | GT73 (RT73) | MON-ØØØ73-7 | Glyphosate tol. | cp4 epsps (aroA:CP4); |
| | | | | goxv247 |
| Canola* | HCN10 (Topas | NA | Glufosinate tol. | bar |
| | 19/2) | | | |
| Canola* | HCN28 (T45) | ACS-BNØØ8-2 | Glufosinate tol. | pat (syn) |
| Canola* | HCN92 (Topas | ACS-BNØØ7-1 | Glufosinate tol. | bar |
| | 19/2) | | | |
| Canola* | MON88302 | MON-883Ø2-9 | Glyphosate tol. | cp4 epsps (aroA:CP4) |
| Canola* | MPS961 | NA | Phytate breakdown | phyA |
| Canola* | MPS962 | NA | Phytate breakdown | phyA |
| Canola* | MPS963 | NA | Phytate breakdown | phyA |
| Canola* | MPS964 | NA | Phytate breakdown | phyA |
| Canola* | MPS965 | NA | Phytate breakdown | phyA |
| Canola* | MS1 (B91-4) | ACS-BNØØ4-7 | Glufosinate tol. | bar |
| Canola* | MS8 | ACS-BNØØ5-8 | Glufosinate tol. | bar |
| Canola* | OXY-235 | ACS-BNØ11-5 | Oxynil tol. | bxn |
| Canola* | PHY14 | NA | Glufosinate tol. | bar |
| Canola* | PHY23 | NA | Glufosinate tol. | bar |
| Canola* | PHY35 | NA | Glufosinate tol. | bar |
| Canola* | PHY36 | NA | Glufosinate tol. | bar |
| Canola* | RF1 (B93-101) | ACS-BNØØ1-4 | Glufosinate tol. | bar |
| Canola* | RF2 (B94-2) | ACS-BNØØ2-5 | Glufosinate tol. | bar |
| Canola* | RF3 | ACS-BNØØ3-6 | Glufosinate tol. | bar |
| Bean | EMBRAPA 5.1 | EMB-PV051-1 | Disease res. | ac1 (sense and |
| | | | | antisense) |
| Brinjal | EE-1 | | Insect res. | cry1Ac |
| (Eggplant) | | | | |
| Carnation | 11 (7442) | FLO-07442-4 | SU tol..; modified flower | surB; dfr; hfl (f3′5′h) |
| | | | color | |
| Carnation | 11363 (1363A) | FLO-11363-1 | SU tol.; modified flower | surB; dfr; bp40 (f3′5′h) |
| | | | color | |
| Carnation | 1226A (11226) | FLO-11226-8 | SU tol.; modified flower | surB; dfr; bp40 (f3′5′h) |
| | | | color | |
| Carnation | 123.2.2 (40619) | FLO-4Ø619-7 | SU tol.; modified flower | surB; dfr; hfl (f3′5′h) |
| | | | color | |
| Carnation | 123.2.38 (40644) | FLO-4Ø644-4 | SU tol.; modified flower | surB; dfr; hfl (f3′5′h) |
| | | | color | |
| Carnation | 123.8.12 | FLO-4Ø689-6 | SU tol.; modified flower | surB; dfr; bp40 (f3′5′h) |
| | | | color | |
| Carnation | 123.8.8 (40685) | FLO-4Ø685-1 | SU tol.; modified flower | surB; dfr; bp40 (f3′5′h) |
| | | | color | |
| Carnation | 1351A (11351) | FLO-11351-7 | SU tol.; modified flower | surB; dfr; bp40 (f3′5′h) |
| | | | color | |
| Carnation | 1400A (11400) | FLO-114ØØ-2 | SU tol.; modified flower | surB; dfr; bp40 (f3′5′h) |
| | | | color | |
| Carnation | 15 | FLO-ØØØ15-2 | SU tol.; modified flower | surB; dfr; hfl (f3′5′h) |
| | | | color | |
| Carnation | 16 | FLO-ØØØ16-3 | SU tol.; modified flower | surB; dfr; hfl (f3′5′h) |
| | | | color | |
| Carnation | 4 | FLO-ØØØØ4-9 | SU tol.; modified flower | surB; dfr; hfl (f3′5′h) |
| | | | color | |
| Carnation | 66 | FLO-ØØØ66-8 | SU tol.; delayed senescence | surB; acc |
| Carnation | 959A (11959) | FLO-11959-3 | SU tol.; modified flower | surB; dfr; bp40 (f3′5′h) |
| | | | color | |
| Carnation | 988A (11988) | FLO-11988-7 | SU tol.; modified flower | surB; dfr; bp40 (f3′5′h) |
| | | | color | |
| Carnation | 26407 | IFD-26497-2 | SU tol.; modified flower | surB; dfr; bp40 (f3′5′h) |
| | | | color | |
| Carnation | 25958 | IFD-25958-3 | SU tol.; modified flower | surB; dfr; bp40 (f3′5′h) |
| | | | color | |
| Chicory | RM3-3 | NA | Glufosinate tol. | bar |
| Chicory | RM3-4 | NA | Glufosinate tol. | bar |
| Chicory | RM3-6 | NA | Glufosinate tol. | bar |
| Cotton | 19-51a | DD-Ø1951A-7 | ALS herbicide tol. | S4-HrA |
| Cotton | 281-24-236 | DAS-24236-5 | Glufosinate tol.; insect res. | pat (syn); cry1F |
| Cotton | 3006-210-23 | DAS-21Ø23-5 | Glufosinate tol.; insect res. | pat (syn); cry1Ac |
| Cotton | 31707 | NA | Oxynil tol.; insect res. | bxn; cry1Ac |
| Cotton | 31803 | NA | Oxynil tol.; insect res. | bxn; cry1Ac |
| Cotton | 31807 | NA | Oxynil tol.; insect res. | bxn; cry1Ac |
| Cotton | 31808 | NA | Oxynil tol.; insect res. | bxn; cry1Ac |
| Cotton | 42317 | NA | Oxynil tol.; insect res. | bxn; cry1Ac |
| Cotton | BNLA-601 | NA | Insect res. | cry1Ac |
| Cotton | BXN10211 | BXN10211-9 | Oxynil tol. | bxn; cry1Ac |
| Cotton | BXN10215 | BXN10215-4 | Oxynil tol. | bxn; cry1Ac |
| Cotton | BXN10222 | BXN10222-2 | Oxynil tol. | bxn; cry1Ac |
| Cotton | BXN10224 | BXN10224-4 | Oxynil tol. | bxn; cry1Ac |
| Cotton | COT102 | SYN-IR102-7 | Insect res. | vip3A(a) |
| Cotton | COT67B | SYN-IR67B-1 | Insect res. | cry1Ab |
| Cotton | COT202 | | Insect res. | vip3A |
| Cotton | Event 1 | NA | Insect res. | cry1Ac |
| Cotton | GMF Cry1A | GTL-GMF311-7 | Insect res. | cry1Ab-Ac |
| Cotton | GHB119 | BCS-GH005-8 | Insect res. | cry2Ae |
| Cotton | GHB614 | BCS-GH002-5 | Glyphosate tol. | 2mepsps |
| Cotton | GK12 | NA | Insect res. | cry1Ab-Ac |
| Cotton | LLCotton25 | ACS-GH001-3 | Glufosinate tol. | bar |
| Cotton | MLS 9124 | NA | Insect res. | cry1C |
| Cotton | MON1076 | MON-89924-2 | Insect res. | cry1Ac |
| Cotton | MON1445 | MON-01445-2 | Glyphosate tol. | cp4 epsps (aroA:CP4) |
| Cotton | MON15985 | MON-15985-7 | Insect res. | cry1Ac; cry2Ab2 |
| Cotton | MON1698 | MON-89383-1 | Glyphosate tol. | cp4 epsps (aroA:CP4) |
| Cotton | MON531 | MON-00531-6 | Insect res. | cry1Ac |
| Cotton | MON757 | MON-00757-7 | Insect res. | cry1Ac |
| Cotton | MON88913 | MON-88913-8 | Glyphosate tol. | cp4 epsps (aroA:CP4) |
| Cotton | Nqwe Chi 6 Bt | NA | Insect res. | NA? |
| Cotton | SKG321 | NA | Insect res. | cry1A; CpTI |
| Cotton | T303-3 | BCS-GH003-6 | Insect res.; glufosinate tol. | cry1Ab; bar |
| Cotton | T304-40 | BCS-GH004-7 | Insect res.; glufosinate tol. | cry1Ab; bar |
| Cotton | CE43-67B | | Insect res. | cry1Ab |
| Cotton | CE46-02A | | Insect res. | cry1Ab |
| Cotton | CE44-69D | | Insect res. | cry1Ab |
| Cotton | 1143-14A | | Insect res. | cry1Ab |
| Cotton | 1143-51B | | Insect res. | cry1Ab |
| Cotton | T342-142 | | Insect res. | cry1Ab |
| Cotton | PV-GHGT07 | | Glyphosate tol. | cp4 epsps (aroA:CP4) |
| | (1445) | | | |
| Cotton | EE-GH3 | | Glyphosate tol. | mepsps |
| Cotton | EE-GH5 | | Insect res. | cry1Ab |
| Cotton | MON88701 | MON-88701-3 | Dicamba & glufosinate tol. | Modified dmo; bar |
| Cotton | OsCr11 | | Anti-allergy | Modified Cry j |
| Creeping | ASR368 | SMG-368ØØ-2 | Glyphosate tol. | cp4 epsps (aroA:CP4) |
| Bentgrass | | | | |
| Eucalyptus | 20-C | | Salt tol. | codA |
| Eucalyptus | 12-5C | | Salt tol. | codA |
| Eucalyptus | 12-5B | | Salt tol. | codA |
| Eucalyptus | 107-1 | | Salt tol. | codA |
| Eucalyptus | Jan. 9, 2001 | | Salt tol. | codA |
| Eucalyptus | Feb. 1, 2001 | | Salt tol. | codA |
| Eucalyptus | | | Cold tol. | des9 |
| Flax | FP967 | CDC-FL001-2 | ALS herbicide tol. | als |
| Lentil | RH44 | | Imidazolinone tol. | als |
| Maize | 3272 | SYN-E3272-5 | Modified alpha-amylase | amy797E |
| Maize | 5307 | SYN-05307-1 | Insect res. | ecry3.1Ab |
| Maize | 59122 | DAS-59122-7 | Insect res.; glufosinate tol. | cry34Ab1; cry35Ab1; |
| | | | | pat |
| Maize | 676 | PH-000676-7 | Glufosinate tol.; pollination | pat; dam |
| | | | control | |
| Maize | 678 | PH-000678-9 | Glufosinate tol.; pollination | pat; dam |
| | | | control | |
| Maize | 680 | PH-000680-2 | Glufosinate tol.; pollination | pat; dam |
| | | | control | |
| Maize | 98140 | DP-098140-6 | Glyphosate toll; ALS | gat4621; zm-hra |
| | | | herbicide tol. | |
| Maize | Bt10 | NA | Insect res.; glufosinate tol. | cry1Ab; pat |
| Maize | Bt176 (176) | SYN-EV176-9 | Insect res.; glufosinate tol. | cry1Ab; bar |
| Maize | BVLA430101 | NA | Phytate breakdown | phyA2 |
| Maize | CBH-351 | ACS-ZM004-3 | Insect res.; glufosinate tol. | cry9C; bar |
| Maize | DAS40278-9 | DAS40278-9 | 2,4-D tol. | aad-1 |
| Maize | DBT418 | DKB-89614-9 | Insect res.; glufosinate tol. | cry1Ac; pinII; bar |
| Maize | DLL25 (B16) | DKB-89790-5 | Glufosinate tol. | bar |
| Maize | GA21 | MON-00021-9 | Glyphosate tol. | mepsps |
| Maize | GG25 | | Glyphosate tol. | mepsps |
| Maize | GJ11 | | Glyphosate tol. | mepsps |
| Maize | Fl117 | | Glyphosate tol. | mepsps |
| Maize | GAT-ZM1 | | Glufosinate tol. | pat |
| Maize | LY038 | REN-00038-3 | Increased lysine | cordapA |
| Maize | MIR162 | SYN-IR162-4 | Insect res. | vip3Aa20 |
| Maize | MIR604 | SYN-IR604-5 | Insect res. | mcry3A |
| Maize | MON801 | MON801 | Insect res.; glyphosate tol. | cry1Ab; cp4 epsps |
| | (MON80100) | | | (aroA:CP4); goxv247 |
| Maize | MON802 | MON-80200-7 | Insect res.; glyphosate tol. | cry1Ab; cp4 epsps |
| | | | | (aroA:CP4); goxv247 |
| Maize | MON809 | PH-MON-809-2 | Insect res.; glyphosate tol. | cry1Ab; cp4 epsps |
| | | | | (aroA:CP4); goxv247 |
| Maize | MON810 | MON-00810-6 | Insect res.; glyphosate tol. | cry1Ab; cp4 epsps |
| | | | | (aroA:CP4); goxv247 |
| Maize | MON832 | NA | Glyphosate tol. | cp4 epsps (aroA:CP4); |
| | | | | goxv247 |
| Maize | MON863 | MON-00863-5 | Insect res. | cry3Bb1 |
| Maize | MON87427 | MON-87427-7 | Glyphosate tol. | cp4 epsps (aroA:CP4) |
| Maize | MON87460 | MON-87460-4 | Drought tol. | cspB |
| Maize | MON88017 | MON-88017-3 | Insect res.; glyphosate tol. | cry3Bb1; cp4 epsps |
| | | | | (aroA:CP4) |
| Maize | MON89034 | MON-89034-3 | Insect res. | cry2Ab2; cry1A.105 |
| Maize | MS3 | ACS-ZM001-9 | Glufosinate tol.; pollination | bar; barnase |
| | | | control | |
| Maize | MS6 | ACS-ZM005-4 | Glufosinate tol.; pollination | bar; barnase |
| | | | control | |
| Maize | NK603 | MON-00603-6 | Glyphosate tol. | cp4 epsps (aroA:CP4) |
| Maize | T14 | ACS-ZM002-1 | Glufosinate tol. | pat (syn) |
| Maize | T25 | ACS-ZM003-2 | Glufosinate tol. | pat (syn) |
| Maize | TC1507 | DAS-01507-1 | Insect res.; glufosinate tol. | cry1Fa2; pat |
| Maize | TC6275 | DAS-06275-8 | Insect res.; glufosinate tol. | mocry1F; bar |
| Maize | VIP1034 | | Insect res.; glufosinate tol. | vip3A; pat |
| Maize | 43A47 | DP-043A47-3 | Insect res.; glufosinate tol. | cry1F; cry34Ab1; |
| | | | | cry35Ab1; pat |
| Maize | 40416 | DP-040416-8 | Insect res.; glufosinate tol. | cry1F; cry34Ab1; |
| | | | | cry35Ab1; pat |
| Maize | 32316 | DP-032316-8 | Insect res.; glufosinate tol. | cry1F; cry34Ab1; |
| | | | | cry35Ab1; pat |
| Maize | 4114 | DP-004114-3 | Insect res.; glufosinate tol. | cry1F; cry34Ab1; |
| | | | | cry35Ab1; pat |
| Melon | Melon A | NA | Delayed ripening/senescence | sam-k |
| Melon | Melon B | NA | Delayed ripening/senescence | sam-k |
| Papaya | 55-1 | CUH-CP551-8 | Disease res. | prsv cp |
| Papaya | 63-1 | CUH-CP631-7 | Disease res. | prsv cp |
| Papaya | Huanong No. 1 | NA | Disease res. | prsv rep |
| Papaya | X17-2 | UFL-X17CP-6 | Disease res. | prsv cp |
| Petunia | Petunia-CHS | NA | Modified product quality | CHS suppres.sion |
| Plum | C-5 | ARS-PLMC5-6 | Disease res. | ppv cp |
| Canola** | ZSR500 | NA | Glyphosate tol. | cp4 epsps (aroA:CP4); |
| | | | | goxv247 |
| Canola** | ZSR502 | NA | Glyphosate tol. | cp4 epsps (aroA:CP4); |
| | | | | goxv247 |
| Canola** | ZSR503 | NA | Glyphosate tol. | cp4 epsps (aroA:CP4); |
| | | | | goxv247 |
| Poplar | Bt poplar | NA | Insect res. | cry1Ac; API |
| Poplar | Hybrid poplar | NA | Insect res. | cry1Ac; API |
| | clone 741 | | | |
| Poplar | trg300-1 | | High cellulose | AaXEG2 |
| Poplar | trg300-2 | | High cellulose | AaXEG2 |
| Potato | 1210 amk | NA | Insect res. | cry3A |
| Potato | 2904/1 kgs | NA | Insect res. | cry3A |
| Canola** | ZSR500 | NA | Glyphosate tol. | cp4 epsps (aroA:CP4); |
| | | | | goxv247 |
| Canola** | ZSR502 | NA | Glyphosate tol. | cp4 epsps (aroA:CP4); |
| | | | | goxv247 |
| Potato | ATBT04-27 | NMK-89367-8 | Insect res. | cry3A |
| Potato | ATBT04-30 | NMK-89613-2 | Insect res. | cry3A |
| Potato | ATBT04-31 | NMK-89170-9 | Insect res. | cry3A |
| Potato | ATBT04-36 | NMK-89279-1 | Insect res. | cry3A |
| Potato | ATBT04-6 | NMK-89761-6 | Insect res. | cry3A |
| Potato | BT06 | NMK-89812-3 | Insect res. | cry3A |
| Potato | BT10 | NMK-89175-5 | Insect res. | cry3A |
| Potato | BT12 | NMK-89601-8 | Insect res. | cry3A |
| Potato | BT16 | NMK-89167-6 | Insect res. | cry3A |
| Potato | BT17 | NMK-89593-9 | Insect res. | cry3A |
| Potato | BT18 | NMK-89906-7 | Insect res. | cry3A |
| Potato | BT23 | NMK-89675-1 | Insect res. | cry3A |
| Potato | EH92-527-1 | BPS-25271-9 | Modified starch/carbohydrate | gbss (antisense) |
| Potato | HLMT15-15 | NA | Insect & disease res. | cry3A; pvy cp |
| Potato | HLMT15-3 | NA | Insect & disease res. | cry3A; pvy cp |
| Potato | HLMT15-46 | NA | Insect & disease res. | cry3A; pvy cp |
| Potato | RBMT15-101 | NMK-89653-6 | Insect & disease res. | cry3A; pvy cp |
| Potato | RBMT21-129 | NMK-89684-1 | Insect & disease res. | cry3A; plrv orf1; plrv |
| | | | | orf2 |
| Potato | RBMT21-152 | NA | Insect & disease res. | cry3A; plrv orf1; plrv |
| | | | | orf2 |
| Potato | RBMT21-350 | NMK-89185-6 | Insect & disease res. | cry3A; plrv orf1; plrv |
| | | | | orf2 |
| Potato | RBMT22-082 | NMK-89896-6 | Insect & disease res.; | cry3A; plrv orf1; plrv |
| | | | Glyphosate tol. | orf2; cp4 epsps |
| | | | | (aroA:CP4) |
| Potato | RBMT22-186 | NA | Insect & disease res.; | cry3A; plrv orf1; plrv |
| | | | Glyphosate tol. | orf2; cp4 epsps |
| | | | | (aroA:CP4) |
| Potato | RBMT22-238 | NA | Insect & disease res.; | cry3A; plrv orf1; plrv |
| | | | Glyphosate tol. | orf2; cp4 epsps |
| | | | | (aroA:CP4) |
| Potato | RBMT22-262 | NA | Insect & disease res.; | cry3A; plrv orf1; plrv |
| | | | Glyphosate tol. | orf2; cp4 epsps |
| | | | | (aroA:CP4) |
| Potato | SEMT15-02 | NMK-89935-9 | Insect & disease res. | cry3A; pvy cp |
| Potato | SEMT15-07 | NA | Insect & disease res. | cry3A; pvy cp |
| Potato | SEMT15-15 | NMK-89930-4 | Insect & disease res. | cry3A; pvy cp |
| Potato | SPBT02-5 | NMK-89576-1 | Insect res. | cry3A |
| Potato | SPBT02-7 | NMK-89724-5 | Insect res. | cry3A |
| Rice | 7Crp#242-95-7 | | Anti-allergy | 7crp |
| Rice | 7Crp#10 | NA | Anti-allergy | 7crp |
| Rice | GM Shanyou 63 | NA | Insect res. | cry1Ab; cry1Ac |
| Rice | Huahui-1/TT51-1 | NA | Insect res. | cry1Ab; cry1Ac |
| Rice | LLRICE06 | ACS-OS001-4 | Glufosinate tol. | bar |
| Rice | LLRICE601 | BCS-OS003-7 | Glufosinate tol. | bar |
| Rice | LLRICE62 | ACS-OS002-5 | Glufosinate tol. | bar |
| Rice | Tarom molaii + | NA | Insect res. | cry1Ab (truncated) |
| | cry1Ab | | | |
| Rice | GAT-OS2 | | Glufosinate tol. | bar |
| Rice | GAT-OS3 | | Glufosinate tol. | bar |
| Rice | PE-7 | | Insect res. | Cry1Ac |
| Rice | 7Crp#10 | NA | Anti-allergy | 7crp |
| Rice | KPD627-8 | | High tryptophan | OASA1D |
| Rice | KPD722-4 | | High tryptophan | OASA1D |
| Rice | KA317 | | High tryptophan | OASA1D |
| Rice | HW5 | | High tryptophan | OASA1D |
| Rice | HW1 | | High tryptophan | OASA1D |
| Rice | B-4-1-18 | | Erect leaves semidwarf | Δ OsBRI1 |
| Rice | G-3-3-22 | | Semidwarf | OSGA2ox1 |
| Rice | AD77 | | Disease res. | DEF |
| Rice | AD51 | | Disease res. | DEF |
| Rice | AD48 | | Disease res. | DEF |
| Rice | AD41 | | Disease res. | DEF |
| Rice | 13pNasNaatAprt1 | | Low iron tol. | HvNAS1; HvNAAT-A; |
| | | | | APRT |
| Rice | 13pAprt1 | | Low iron tol. | APRT |
| Rice | gHvNAS1- | | Low iron tol. | HvNAS1; HvNAAT-A; |
| | gHvNAAT-1 | | | HvNAAT-B |
| Rice | gHvIDS3-1 | | Low iron tol. | HvIDS3 |
| Rice | gHvNAAT1 | | Low iron tol. | HvNAAT-A; |
| | | | | HvNAAT-B |
| Rice | gHvNAS1-1 | | Low iron tol. | HvNAS1 |
| Rice | NIA-OS006-4 | | Disease res. | WRKY45 |
| Rice | NIA-OS005-3 | | Disease res. | WRKY45 |
| Rice | NIA-OS004-2 | | Disease res. | WRKY45 |
| Rice | NIA-OS003-1 | | Disease res. | WRKY45 |
| Rice | NIA-OS002-9 | | Disease res. | WRKY45 |
| Rice | NIA-OS001-8 | | Disease res. | WRKY45 |
| Rice | OsCr11 | | Anti-allergy | Modified Cry j |
| Rice | 17053 | | Glyphosate tol. | cp4 epsps (aroA:CP4) |
| Rice | 17314 | | Glyphosate tol. | cp4 epsps (aroA:CP4) |
| Rose | WKS82/130-4-1 | IFD-52401-4 | Modified flower color | 5AT; bp40 (f3′5′h) |
| Rose | WKS92/130-9-1 | IFD-52901-9 | Modified flower color | 5AT; bp40 (f3′5′h) |
| Soybean | 260-05 (G94-1, | NA | Modified oil/fatty acid | gm-fad2-1 (silencing |
| | G94-19, G168) | | | locus) |
| Soybean | A2704-12 | ACS-GM005-3 | Glufosinate tol. | pat |
| Soybean | A2704-21 | ACS-GM004-2 | Glufosinate tol. | pat |
| Soybean | A5547-127 | ACS-GM006-4 | Glufosinate tol. | pat |
| Soybean | A5547-35 | ACS-GM008-6 | Glufosinate tol. | pat |
| Soybean | CV127 | BPS-CV127-9 | Imidazolinone tol. | csr1-2 |
| Soybean | DAS68416-4 | DAS68416-4 | Glufosinate tol. | pat |
| Soybean | DP305423 | DP-305423-1 | Modified oil/fatty acid; | gm-fad2-1 (silencing |
| | | | ALS herbicide tol. | locus); gm-hra |
| Soybean | DP356043 | DP-356043-5 | Modified oil/fatty acid; | gm-fad2-1 (silencing |
| | | | glyphosate tol. | locus); gat4601 |
| Soybean | FG72 | MST-FG072-3 | Glyphosate & HPPD tol. | 2mepsps; hppdPF |
| | | | | W336 |
| Soybean | GTS 40-3-2 | MON-04032-6 | Glyphosate tol. | cp4 epsps (aroA:CP4) |
| | (40-3-2) | | | |
| Soybean | GU262 | ACS-GM003-1 | Glufosinate tol. | pat |
| Soybean | MON87701 | MON-87701-2 | Insect res. | cry1Ac |
| Soybean | MON87705 | MON-87705-6 | Modified oil/fatty acid; | fatb1-A (sense & |
| | | | glyphosate tol. | antisense); fad2-1A |
| | | | | (sense & antisense); |
| | | | | cp4 epsps (aroA:CP4) |
| Soybean | MON87708 | MON-87708-9 | Dicamba & glyphosate tol. | dmo; cp4 epsps |
| | | | | (aroA:CP4) |
| Soybean | MON87769 | MON-87769-7 | Modified oil/fatty acid; | Pj.D6D; Nc.Fad3; cp4 |
| | | | glyphosate tol. | epsps (aroA:CP4) |
| Soybean | MON89788 | MON-89788-1 | Glyphosate tol. | cp4 epsps (aroA:CP4) |
| Soybean | W62 | ACS-GM002-9 | Glufosinate tol. | bar |
| Soybean | W98 | ACS-GM001-8 | Glufosinate tol. | bar |
| Soybean | MON87754 | MON-87754-1 | High oil | dgat2A |
| Soybean | DAS21606 | DAS-21606 | Aryloxyalkanoate & | Modified aad-12; pat |
| | | | glufosinate tol. | |
| Soybean | DAS44406 | DAS-44406-6 | Aryloxyalkanoate, glyphosate | Modified aad-12; |
| | | | & glufosinate tol. | 2mepsps; pat |
| Soybean | SYHT04R | SYN-0004R-8 | Mesotrione tol. | Modified avhppd |
| Soybean | 9582.814.19.1 | | Insect res. & glufosinate tol. | cry1Ac, cry1F, PAT |
| Squash | CZW3 | SEM-ØCZW3-2 | Disease res. | cmv cp, zymv cp, wmv |
| | | | | cp |
| Squash | ZW20 | SEM-0ZW20-7 | Disease res. | zymv cp, wmv cp |
| Sugar Beet | GTSB77 | SY-GTSB77-8 | Glyphosate tol. | cp4 epsps (aroA:CP4); |
| | (T9100152) | | | goxv247 |
| Sugar Beet | H7-1 | KM-000H71-4 | Glyphosate tol. | cp4 epsps (aroA:CP4) |
| Sugar Beet | T120-7 | ACS-BV001-3 | Glufosinate tol. | pat |
| Sugar Beet | T227-1 | | Glyphosate tol. | cp4 epsps (aroA:CP4) |
| Sugarcane | NXI-1T | | Drought tol. | EcbetA |
| Sunflower | X81359 | | Imidazolinone tol. | als |
| Sweet | PK-SP01 | NA | Disease res. | cmv cp |
| Pepper | | | | |
| Tobacco | C/F/93/08-02 | NA | Oxynil tol. | bxn |
| Tobacco | Vector 21-41 | NA | Reduced nicotine | NtQPT1 (antisense) |
| Tomato | 1345-4 | NA | Delayed ripening/senescense | acc (truncated) |
| Tomato | 35-1-N | NA | Delayed ripening/senescense | sam-k |
| Tomato | 5345 | NA | Insect res. | cry1Ac |
| Tomato | 8338 | CGN-89322-3 | Delayed ripening/senescense | accd |
| Tomato | B | SYN-0000B-6 | Delayed ripening/senescense | pg (sense or antisense) |
| Tomato | Da | SYN-0000DA-9 | Delayed ripening/senescense | pg (sense or antisense) |
| Sunflower | X81359 | | Imidazolinone tol. | als |
| Tomato | Da Dong No 9 | NA | Modified product | NA |
| Tomato | F (1401F, h38F, | SYN-0000F-1 | Delayed ripening/senescense | pg (sense or antisense) |
| | 11013F, 7913F) | | | |
| Tomato | FLAVR SAVR ™ | CGN-89564-2 | Delayed ripening/senescense | pg (sense or antisense) |
| Tomato | Huafan No 1 | NA | Delayed ripening/senescense | anti-efe |
| Tomato | PK-TM8805R | NA | Disease res. | cmv cp |
| | (8805R) | | | |
| Wheat | MON71800 | MON-718ØØ-3 | Glyphosate tol. | cp4 epsps (aroA:CP4) |

*Argentine,
**Polish,
# Eggplant

Treatment of genetically modified plants and seeds with compounds of the disclosure may result in enhanced effects. For example, reduction in application rates, broadening of the activity spectrum, increased tolerance to biotic/abiotic stresses or enhanced storage stability may be greater than expected from just simple additive effects of the application of compounds of the disclosure on genetically modified plants and seeds.

Compounds of this disclosure are also useful in seed treatments for protecting seeds from invertebrate pests. In the context of the present disclosure and claims, treating a seed means contacting the seed with a biologically effective amount of a compound of this disclosure, which is typically formulated as a composition of the disclosure. This seed treatment protects the seed from invertebrate soil pests and generally can also protect roots and other plant parts in contact with the soil of the seedling developing from the germinating seed. The seed treatment may also provide protection of foliage by translocation of the compound of this disclosure or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this disclosure can also increase vigor of plants growing from the treated seed.

One method of seed treatment is by spraying or dusting the seed with a compound of the disclosure (i.e. as a formulated composition) before sowing the seeds. Compositions formulated for seed treatment generally comprise a film former or adhesive agent. Therefore, typically a seed coating composition of the present disclosure comprises a biologically effective amount of a compound of Formula 1, an N-oxide or salt thereof, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

Compounds of Formula 1 or Formula 1′ and their compositions, both alone and in combination with other insecticides and fungicides, are particularly useful in seed treatment for crops including, but not limited to, maize or corn, soybeans, cotton, cereal (e.g., wheat, oats, barley, rye and rice), potatoes, vegetables and oilseed rape.

Other insecticides with which compounds of Formula 1 or Formula 1′ can be formulated to provide mixtures useful in seed treatment include abamectin, acetamiprid, acrinathrin, amitraz, avermectin, azadirachtin, bensultap, bifenthrin, buprofezin, carbaryl, carbofuran, cartap, chlorantraniliprole, chlorfenapyr, chlorpyrifos, clothianidin, cyantraniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, flufenoxuron, fluvalinate, formetanate, fosthiazate, hexaflumuron, hydramethylnon, imidacloprid, indoxacarb, lufenuron, metaflumizone, methiocarb, methomyl, methoprene, methoxyfenozide, nitenpyram, nithiazine, novaluron, oxamyl, pymetrozine, pyrethrin, pyridaben, pyridalyl, pyriproxyfen, ryanodine, spinetoram, spinosad, spirodiclofen, spiromesifen, spirotetramat, sulfoxaflor, tebufenozide, tetramethrin, thiacloprid, thiamethoxam, thiodicarb, thiosultap-sodium, tralomethrin, triazamate, triflumuron, Bacillus thuringiensis delta-endotoxins, all strains of Bacillus thuringiensis and all strains of nuclear polyhedrosis viruses.

Fungicides with which compounds of Formula 1 or Formula 1′ can be formulated to provide mixtures useful in seed treatment include amisulbrom, azoxystrobin, boscalid, carbendazim, carboxin, cymoxanil, cyproconazole, difenoconazole, dimethomorph, fluazinam, fludioxonil, fluquinconazole, fluopicolide, fluoxastrobin, flutriafol, fluxapyroxad, ipconazole, iprodione, metalaxyl, mefenoxam, metconazole, myclobutanil, paclobutrazole, penflufen, picoxystrobin, prothioconazole, pyraclostrobin, sedaxane, silthiofam, tebuconazole, thiabendazole, thiophanate-methyl, thiram, trifloxystrobin and triticonazole.

Compositions comprising compounds of Formula 1 useful for seed treatment can further comprise bacteria such as Bacillus pumilus (e.g., strain GB34) and Bacillus firmus (e.g., isolate 1582), rhizobia inoculants/extenders, isoflavonoids and lipo-chitooligosaccharides.

The treated seed typically comprises a compound of the present disclosure in an amount from about 0.1 g to 1 kg per 100 kg of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this disclosure can be incorporated into a bait composition that is consumed by an invertebrate pest or used within a device such as a trap, bait station, and the like. Such a bait composition can be in the form of granules which comprise (a) active ingredients, namely a biologically effective amount of a compound of Formula 1, an N-oxide, or salt thereof; (b) one or more food materials; optionally (c) an attractant, and optionally (d) one or more humectants. Of note are granules or bait compositions which comprise between about 0.001-5% active ingredients, about 40-99% food material and/or attractant; and optionally about 0.05-10% humectants, which are effective in controlling soil invertebrate pests at very low application rates, particularly at doses of active ingredient that are lethal by ingestion rather than by direct contact. Some food materials can function both as a food source and an attractant. Food materials include carbohydrates, proteins and lipids. Examples of food materials are vegetable flour, sugar, starches, animal fat, vegetable oil, yeast extracts and milk solids. Examples of attractants are odorants and flavorants, such as fruit or plant extracts, perfume, or other animal or plant component, pheromones or other agents known to attract a target invertebrate pest. Examples of humectants, i.e. moisture retaining agents, are glycols and other polyols, glycerine and sorbitol. Of note is a bait composition (and a method utilizing such a bait composition) used to control at least one invertebrate pest selected from the group consisting of ants, termites and cockroaches. A device for controlling an invertebrate pest can comprise the present bait composition and a housing adapted to receive the bait composition, wherein the housing has at least one opening sized to permit the invertebrate pest to pass through the opening so the invertebrate pest can gain access to the bait composition from a location outside the housing, and wherein the housing is further adapted to be placed in or near a locus of potential or known activity for the invertebrate pest.

The compounds of this disclosure can be applied without other adjuvants, but most often application will be of a formulation comprising one or more active ingredients with suitable carriers, diluents, and surfactants and possibly in combination with a food depending on the contemplated end use. One method of application involves spraying a water dispersion or refined oil solution of a compound of the present disclosure. Combinations with spray oils, spray oil concentrations, spreader stickers, adjuvants, other solvents, and piperonyl butoxide often enhance compound efficacy. For nonagronomic uses such sprays can be applied from spray containers such as a can, a bottle or other container, either by means of a pump or by releasing it from a pressurized container, e.g., a pressurized aerosol spray can. Such spray compositions can take various forms, for example, sprays, mists, foams, fumes or fog. Such spray compositions thus can further comprise propellants, foaming agents, etc. as the case may be. Of note is a spray composition comprising a biologically effective amount of a compound or a composition of the present disclosure and a carrier. One embodiment of such a spray composition comprises a biologically effective amount of a compound or a composition of the present disclosure and a propellant. Representative propellants include, but are not limited to, methane, ethane, propane, butane, isobutane, butene, pentane, isopentane, neopentane, pentene, hydrofluorocarbons, chlorofluorocarbons, dimethyl ether, and mixtures of the foregoing. Of note is a spray composition (and a method utilizing such a spray composition dispensed from a spray container) used to control at least one invertebrate pest selected from the group consisting of mosquitoes, black flies, stable flies, deer flies, horse flies, wasps, yellow jackets, hornets, ticks, spiders, ants, gnats, and the like, including individually or in combinations.

One embodiment of the present disclosure relates to a method for controlling invertebrate pests, comprising diluting the pesticidal composition of the present disclosure (a compound of Formula 1 or Formula 1′ formulated with surfactants, solid diluents and liquid diluents or a formulated mixture of a compound of Formula 1 or Formula 1′ and at least one other pesticide) with water, and optionally adding an adjuvant to form a diluted composition, and contacting the invertebrate pest or its environment with an effective amount of said diluted composition.

Although a spray composition formed by diluting with water a sufficient concentration of the present pesticidal composition can provide sufficient efficacy for controlling invertebrate pests, separately formulated adjuvant products can also be added to spray tank mixtures. These additional adjuvants are commonly known as “spray adjuvants” or “tank-mix adjuvants”, and include any substance mixed in a spray tank to improve the performance of a pesticide or alter the physical properties of the spray mixture. Adjuvants can be surfactants, emulsifying agents, petroleum-based crop oils, crop-derived seed oils, acidifiers, buffers, thickeners or defoaming agents. Adjuvants are used to enhancing efficacy (e.g., biological availability, adhesion, penetration, uniformity of coverage and durability of protection), or minimizing or eliminating spray application problems associated with incompatibility, foaming, drift, evaporation, volatilization and degradation. To obtain optimal performance, adjuvants are selected with regard to the properties of the active ingredient, formulation and target (e.g., crops, insect pests).

Among the spray adjuvants, oils including crop oils, crop oil concentrates, vegetable oil concentrates and methylated seed oil concentrates are most commonly used to improve the efficacy of pesticides, possibly by means of promoting more even and uniform spray deposits. In situations where phytotoxicity potentially caused by oils or other water-immiscible liquids are of concern, spray compositions prepared from the composition of the present disclosure will generally not contain oil-based spray adjuvants. However, in situations where phytotoxicity caused by oil-based spray adjuvants is commercially insignificant, spray compositions prepared from the composition of the present composition can also contain oil-based spray adjuvants, which can potentially further increase control of invertebrate pests, as well as rainfastness.

Products identified as “crop oil” typically contain 95 to 98% paraffin or naphtha-based petroleum oil and 1 to 2% of one or more surfactants functioning as emulsifiers. Products identified as “crop oil concentrates” typically consist of 80 to 85% of emulsifiable petroleum-based oil and 15 to 20% of nonionic surfactants. Products correctly identified as “vegetable oil concentrates” typically consist of 80 to 85% of vegetable oil (i.e. seed or fruit oil, most commonly from cotton, linseed, soybean or sunflower) and 15 to 20% of nonionic surfactants. Adjuvant performance can be improved by replacing the vegetable oil with methyl esters of fatty acids that are typically derived from vegetable oils. Examples of methylated seed oil concentrates include MSO® Concentrate (UAP-Loveland Products, Inc.) and Premium MSO Methylated Spray Oil (Helena Chemical Company).

The amount of adjuvants added to spray mixtures generally does not exceed about 2.5% by volume, and more typically the amount is from about 0.1 to about 1% by volume. The application rates of adjuvants added to spray mixtures are typically between about 1 to 5 L per hectare. Representative examples of spray adjuvants include: Adigor® (Syngenta) 47% methylated rapeseed oil in liquid hydrocarbons, Silwet® (Helena Chemical Company) polyalkyleneoxide modified heptamethyltrisiloxane and Assist® (BASF) 17% surfactant blend in 83% paraffin based mineral oil.

Nonagronomic applications include protecting an animal, particularly a vertebrate, more particularly a homeothermic vertebrate (e.g., mammal or bird) and most particularly a mammal, from an invertebrate parasitic pest by administering a parasiticidally effective (i.e. biologically effective) amount of a compound of the disclosure, typically in the form of a composition formulated for veterinary use, to the animal to be protected. Therefore of note is a method for protecting an animal comprising administering to the animal a parasiticidally effective amount of a compound of the disclosure. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include necrosis, death, retarded growth, diminished mobility or lessened ability to remain on or in the host animal, reduced feeding and inhibition of reproduction. These effects on invertebrate parasite pests provide control (including prevention, reduction or elimination) of parasitic infestation or infection of the animal. Examples of invertebrate parasitic pests controlled by administering a parasiticidally effective amount of a compound of the disclosure to an animal to be protected include ectoparasites (arthropods, acarines, etc) and endoparasites (helminths, e.g., nematodes, trematodes, cestodes, acanthocephalans, etc.). In particular, the compounds of this disclosure are effective against ectoparasites including: flies such as Haematobia (Lyperosia) irritans (horn fly), Stomoxys calcitrans (stable fly), Simulium spp. (blackfly), Glossina spp. (tsetse flies), Hydrotaea irritans (head fly), Musca autumnalis (face fly), Musca domestica (house fly), Morellia simplex (sweat fly), Tabanus spp. (horse fly), Hypoderma bovis, Hypoderma lineatum, Lucilia sericata, Lucilia cuprina (green blowfly), Calliphora spp. (blowfly), Protophormia spp., Oestrus ovis (nasal botfly), Culicoides spp. (midges), Hippobosca equine, Gastrophilus instestinalis, Gastrophilus haemorrhoidalis and Gastrophilus naslis; lice such as Bovicola (Damalinia) bovis, Bovicola equi, Haematopinus asini, Felicola subrostratus, Heterodoxus spiniger, Lignonathus setosus and Trichodectes canis; keds such as Melophagus ovinus; mites such as Psoroptes spp., Sarcoptes scabei, Chorioptes bovis, Demodex equi, Cheyletiella spp., Notoedres cati, Trombicula spp. and Otodectes cyanotis (ear mites); ticks such as Ixodes spp., Boophilus spp., Rhipicephalus spp., Amblyomma spp., Dermacentor spp., Hyalomma spp. and Haemaphysalis spp.; and fleas such as Ctenocephalides felis (cat flea) and Ctenocephalides canis (dog flea).

Nonagronomic applications in the veterinary sector are by conventional means such as by enteral administration in the form of, for example, tablets, capsules, drinks, drenching preparations, granulates, pastes, boli, feed-through procedures, or suppositories; or by parenteral administration, such as by injection (including intramuscular, subcutaneous, intravenous, intraperitoneal) or implants; by nasal administration; by topical administration, for example, in the form of immersion or dipping, spraying, washing, coating with powder, or application to a small area of the animal, and through articles such as neck collars, ear tags, tail bands, limb bands or halters which comprise compounds or compositions of the present disclosure.

Typically a parasiticidal composition according to the present disclosure comprises a mixture of a compound of Formula 1 or Formula 1′, an N-oxide or a salt thereof, with one or more pharmaceutically or veterinarily acceptable carriers comprising excipients and auxiliaries selected with regard to the intended route of administration (e.g., oral, topical or parenteral administration such as injection) and in accordance with standard practice. In addition, a suitable carrier is selected on the basis of compatibility with the one or more active ingredients in the composition, including such considerations as stability relative to pH and moisture content. Therefore of note is a composition for protecting an animal from an invertebrate parasitic pest comprising a parasitically effective amount of a compound of the disclosure and at least one carrier.

For parenteral administration including intravenous, intramuscular and subcutaneous injection, a compound of the present disclosure can be formulated in suspension, solution or emulsion in oily or aqueous vehicles, and may contain adjuncts such as suspending, stabilizing and/or dispersing agents. Pharmaceutical compositions for injection include aqueous solutions of water-soluble forms of active ingredients (e.g., a salt of an active compound), preferably in physiologically compatible buffers containing other excipients or auxiliaries as are known in the art of pharmaceutical formulation.

For oral administration in the form of solutions (the most readily available form for absorption), emulsions, suspensions, pastes, gels, capsules, tablets, boluses powders, granules, rumen-retention and feed/water/lick blocks, a compound of the present disclosure can be formulated with binders/fillers known in the art to be suitable for oral administration compositions, such as sugars (e.g., lactose, sucrose, mannitol, sorbitol), starch (e.g., maize starch, wheat starch, rice starch, potato starch), cellulose and derivatives (e.g., methylcellulose, carboxymethylcellulose, ethylhydroxycellulose), protein derivatives (e.g., zein, gelatin), and synthetic polymers (e.g., polyvinyl alcohol, polyvinylpyrrolidone). If desired, lubricants (e.g., magnesium stearate), disintegrating agents (e.g., cross-linked polyvinylpyrrolidinone, agar, alginic acid) and dyes or pigments can be added. Pastes and gels often also contain adhesives (e.g., acacia, alginic acid, bentonite, cellulose, xanthan gum, colloidal magnesium aluminum silicate) to aid in keeping the composition in contact with the oral cavity and not being easily ejected.

If the parasiticidal compositions are in the form of feed concentrates, the carrier is typically selected from high-performance feed, feed cereals or protein concentrates. Such feed concentrate-containing compositions can, in addition to the parasiticidal active ingredients, comprise additives promoting animal health or growth, improving quality of meat from animals for slaughter or otherwise useful to animal husbandry. These additives can include, for example, vitamins, antibiotics, chemotherapeutics, bacteriostats, fungistats, coccidiostats and hormones.

Compounds of the present disclosure have been discovered to have favorable pharmacokinetic and pharmacodynamic properties providing systemic availability from oral administration and ingestion. Therefore after ingestion by the animal to be protected, parasiticidally effective concentrations of compounds of the disclosure in the bloodstream protect the treated animal from blood-sucking pests such as fleas, ticks and lice. Therefore of note is a composition for protecting an animal from an invertebrate parasite pest in a form for oral administration (i.e. comprising, in addition to a parasiticidally effective amount of a compound of the disclosure, one or more carriers selected from binders and fillers suitable for oral administration and feed concentrate carriers).

Formulations for topical administration are typically in the form of a powder, cream, suspension, spray, emulsion, foam, paste, aerosol, ointment, salve or gel. More typically a topical formulation is a water-soluble solution, which can be in the form of a concentrate that is diluted before use. Parasiticidal compositions suitable for topical administration typically comprise a compound of the present disclosure and one or more topically suitable carriers. In applications of a parasiticidal composition topically to the exterior of an animal as a line or spot (i.e. “spot-on” treatment), the active ingredient migrates over the surface of the animal to cover most or all of its external surface area. As a result, the treated animal is particularly protected from invertebrate pests that feed off the epidermis of the animal such as ticks, fleas and lice. Therefore formulations for topical localized administration often comprise at least one organic solvent to facilitate transport of the active ingredient over the skin and/or penetration into the epidermis of the animal. Solvents commonly used as carriers in such formulations include propylene glycol, paraffins, aromatics, esters such as isopropyl myristate, glycol ethers, and alcohols such as ethanol and n-propanol.

The rate of application required for effective control (i.e. “biologically effective amount”) will depend on such factors as the species of invertebrate to be controlled, the pest's life cycle, life stage, its size, location, time of year, host crop or animal, feeding behavior, mating behavior, ambient moisture, temperature, and the like. Under normal circumstances, application rates of about 0.01 to 2 kg of active ingredients per hectare are sufficient to control pests in agronomic ecosystems, but as little as 0.0001 kg/hectare may be sufficient or as much as 8 kg/hectare may be required. For nonagronomic applications, effective use rates will range from about 1.0 to 50 mg/square meter but as little as 0.1 mg/square meter may be sufficient or as much as 150 mg/square meter may be required. One skilled in the art can easily determine the biologically effective amount necessary for the desired level of invertebrate pest control.

In general, for veterinary use, a compound of Formula 1 or Formula 1′, an N-oxide or a salt thereof, is administered in a parasiticidally effective amount to an animal to be protected from invertebrate parasite pests. A parasiticidally effective amount is the amount of active ingredient needed to achieve an observable effect diminishing the occurrence or activity of the target invertebrate parasite pest. One skilled in the art will appreciate that the parasitically effective dose can vary for the various compounds and compositions of the present disclosure, the desired parasitical effect and duration, the target invertebrate pest species, the animal to be protected, the mode of application and the like, and the amount needed to achieve a particular result can be determined through simple experimentation.

For oral administration to homeothermic animals, the daily dosage of a compound of the present disclosure typically ranges from about 0.01 mg/kg to about 100 mg/kg, more typically from about 0.5 mg/kg to about 100 mg/kg, of animal body weight. For topical (e.g., dermal) administration, dips and sprays typically contain from about 0.5 ppm to about 5000 ppm, more typically from about 1 ppm to about 3000 ppm, of a compound of the present disclosure.

Recent advances in the processing power of computers have afforded scientists unprecedented opportunities to leverage in silico tools to predict and investigate potential adverse outcomes associated with xenobiotic exposures, along with the molecular bases for these events. While current computational models cannot solely be used to replace all in vivo or in vitro experimental approaches, they nevertheless provide valuable tools to generate hypotheses, flag compounds of interest, and help prioritize and align chemicals to appropriate in vitro or in vivo studies.

Computational/predictive toxicology is a rapidly developing discipline that integrates information and data from a variety of sources to develop mathematical and computer-based models to better understand and predict interactions of chemical agents and biological organisms across many scales (e.g., population, individual, cellular, and molecular) and can be considered to encompass two broad areas:

In one aspect, the development and application of 2-dimensional (2D) models via first principles (e.g., structural motifs that drive facile chemical reactivity (Wijeyesakere, S. J. et al. Development of a Profiler for Facile Chemical Reactivity Using the Open-Source Konstanz Information Miner. Appl. Vitr. Toxicol., 4, 202-213, 2018). Additionally, in order to predict the dosimetry for a biological outcome of interest, quantitative structure-activity relationship techniques (QSAR) such as tend-analysis may be undertaken if experimental data from analogous molecules is available (e.g. prediction of the cholinergic potential of a series of related organophosphorus compounds (Makhaeva, G. F. et al. Esterase profiles of organophosphorus compounds in vitro predict their behavior in vivo. Chem. Biol. Interact., 259, 332-342, 2016). Makhaeva, G. F. et al. Kinetics and mechanism of inhibition of serine esterases by fluorinated carbethoxy 1-aminophosphonates. Dokl. Biochem. Biophys., 451, 203-206, 2013).

In another aspect, the use of “big data” approaches that curate all available information on a particular outcome/mode of action (e.g., predictive models to identify mitochondrial inhibitors (Wijeyesakere, S. J. et al. Hybrid Machine-Learning/SMARTS Profiling Model for Mitochondrial Inhibition. Appl. Vitr. Toxicol., 5, 196-204, 2019) or those that can interact with defined neuronal receptors (Wijeyesakere, S. J. et al. Prediction of cholinergic compounds by machine-learning. Comput. Toxicol., 13, 100119, 2020); and extension of these 2D assessments to encompass 3D techniques such as docking and molecular dynamics (MD) simulations to further investigate the molecular basis for interactions between toxicants and known/putative biological targets (Wang, Y. et al. Mixed inhibition of adenosine deaminase activity by 1,3-dinitrobenzene: A Model for understanding cell-selective neurotoxicity in chemically-induced energy deprivation syndromes in brain. Toxicol. Sci., 125, 509-521, 2012; Gonzalez, T. L. et al. Metabolites of n-Butylparaben and iso-Butylparaben Exhibit Estrogenic Properties in MCF-7 and T47D Human Breast Cancer Cell Lines. Toxicol. Sci., 164, 50-59018, 2018)

The compounds of this disclosure prepared by the methods described herein are shown in Index Table A. For mass spectral (MS) data, the numerical value reported is the molecular weight of the highest isotopic abundance parent ion (M+1) formed by addition of H⁺ (molecular weight of 1) to the molecule, observed by mass spectrometry using atmospheric pressure chemical ionization (AP⁺). The following abbreviations are used in the Index Table which follows: Cmpd means Compound, i-Pr means isopropyl, Bu means butyl, c-Pr means cyclopropyl, c-Bu means cyclobutyl, t-Bu means tert-butyl, Et means ethyl. The abbreviation “Ex.” stands for “Example” and is followed by a number indicating in which Synthesis Example the compound is prepared.

Specific compounds of Formula 1 or Formula 1′, prepared by the methods and variations as described in preceding Schemes 1-13 and Synthesis Example 1, are shown in the Index Tables below. The following abbreviations may be used: Cmpd means Compound, t is tertiary, c is cyclo, Me is methyl, Et is ethyl and Ph is phenyl. The abbreviation “Ex.” stands for “Example” and is followed by a number indicating in which Synthesis Example the compound is prepared. For mass spectral data (AP⁺ (M+1)), the numerical value reported is the molecular weight of the parent molecular ion (M) formed by addition of H⁺ (molecular weight of 1) to the molecule to give a M+1 peak observed by mass spectrometry using atmospheric pressure chemical ionization (AP⁺). The alternate molecular ion peaks (e.g., M+2 or M+4) that occur with compounds containing multiple halogens are not reported.

INDEX TABLE A

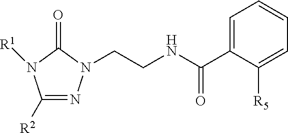

| Cmpd. | | | | |
|---|---|---|---|---|
| No. | R¹ | R² | R⁵ | MS (M + 1) |
| 1 | —CH₂CF₃ | —CF₃ | —CF₃ | 451 |
| 2 | —c-Pr | —CF₃ | —CF₃ | 409 |
| 3 | —c-Bu | —CF₃ | —Cl | 389 |
| 4 | —c-Bu | —CF₃ | —CF₃ | 423 |
| 5 | —Me | —CHF₂ | —Cl | 331 |
| 6 | —Me | —CF₃ | —Cl | 349 |
| 7 | —Me | —CF₂CF₃ | —Cl | 399 |
| 8 | —Me | —CF₂CF₃ | —CF₃ | 433 |
| 9 | —i-Pr | —CHF₂ | —CF₃ | 393 |
| 10 | —t-Bu | —CF₃ | —CF₃ | 425 |
| 11 | —i-Pr | —CF₃ | —CF₃ | 411 |
| 12 | —Et | —CF₃ | —CF₃ | 397 |
| 13 | —CHF₂ | —CF₃ | —CF₃ | 330 |
| 14 | —Me | —CF₃ | —CF₃ | 382 |
| 15 | —Me | —CHF₂ | —CF₃ | 365 |
| 16 | —CH₂CN | —CF₃ | —Cl | 373 |
| 17 | —CH₂CN | —CF₃ | —CF₃ | 407 |
| 18 | —CH(Et)₂ | —CF₃ | —CF₃ | 439 |
| 19 | —CH(Et)₂ | —CF₃ | —Cl | 405 |
| 20 | —CH₂(cyclopentyl) | —CF₃ | —Cl | 417 |
| 21 | —CH₂(cyclopentyl) | —CF₃ | —CF₃ | 451 |
| 22 | cyclopentyl | —CF₃ | —Cl | 403 |
| 23 | cyclopentyl | —CF₃ | —CF₃ | 437 |
| 24 | —i-Pr(CF₃ | —CF₃ | —CF₃ | 479 |
| 25 | —i-Pr(Et) | —CF₃ | —CF₃ | 439 |
| 26 | —i-Pr(Et) | —CF₃ | —Cl | 405 |
| 27 | —CH(Me)c-Pr | —CF₃ | —CF₃ | 437 |
| 28 | —CH(Me)c-Pr | —CF₃ | —Cl | 403 |
| 29 | —CH₂c-Pr | —CF₃ | —Cl | 389 |
| 30 | —CH₂CF₃ | —CF₃ | —Cl | 417 |
| 31 | —i-Pr | —CF₃ | —Cl | 377 |
| 32 | —CH2c-Pr | —CF₃ | —CF₃ | 423 |
| 33 | —CH₂CH═CH₂ | —CF₃ | —CF₃ | 409 |
| 34 | —CH₂CH₂c-Pr | —CF₃ | —Cl | 403 |
| 35 | —CH₂CH₂c-Pr | —CF₃ | —CF₃ | 437 |
| 36 | —CH₂c-Bu | —CF₃ | —Cl | 403 |
| 37 | —CH₂c-Bu | —CF₃ | —CF₃ | 437 |
| 38 | —t-Bu | —CF₃ | —Cl | 391 |
| 39 | —CH₂CH₂OMe | —CF₃ | —Cl | 393 |
| 40 | —CH₂CH₂OMe | —CF₃ | —CF₃ | 427 |
| 41 | —CH₂CH═CH₂ | —CF₃ | —Cl | 375 |
| 42 | —CH₂CH₂CH₂Me | —CF₃ | —Cl | 391 |
| 43 | —CH₂CH₂CH₂Me | —CF₃ | —CF₃ | 425 |
| 44 | 1-methylcyclopropyl | —CF₃ | —Cl | 389 |
| 45 | 1-methylcyclopropyl | —CF₃ | —CF₃ | 423 |
| 46 | —CH₂(i-Bu) | —CF₃ | —Cl | 405 |
| 47 | —CH₂(i-Bu) | —CF₃ | —CF₃ | 439 |
| 48 | —Me | —CF₂CF₃ | —F | 383 |
| 49 | —Me | —CF₂CF₃ | —Br | 444 |
| 50 | —t-Bu | —CF₃ | —Br | 436 |
| 51 | —t-Bu | —CF₃ | —Me | 371 |
| 52 | —CH₂(cyclohexyl) | —CF₃ | —CF₃ | 465 |
| 53 | —CH₂(cyclohexyl) | —CF₃ | —Cl | 431 |
| 54 | —Me | —CF₂CF₂CF₃ | —Cl | 449 |
| 55 | —CH₂CHF₂ | —CF₃ | —Cl | 399 |
| 56 | —CH₂CF₂ | —CF₃ | —CF₃ | 433 |
| 57 | —i-Bu | —CF₃ | —Cl | 391 |
| 58 | —i-Bu | —CF₃ | —CF₃ | 425 |
| 59 | —c-Pr | —CF₃ | —Cl | 375 |
| 60 | —CH₂CF₂ | —CF₃ | —CF₃ | 434 |
| 61 | —t-Bu | —CF₃ | —CF₃ | 439 |
| 62 | —t-Bu | —CF₃ | —Cl | 405 |
| 63 | —CH(Me)CF₃ | —CF₃ | —Cl | 431 |
| 64 | —CH(Me)CF₃ | —CF₃ | —CF₃ | 465 |
| 65 | —CH₂(t-Bu) | —CF₃ | —CF₃ | 439 |
| 66 | —CH₂(t-Bu) | —CF₃ | —Cl | 405 |
| 67 | —CH₂C(O)Me | —CF₃ | —Cl | 390 |
| 68 | —CH₂C(O)Me | —CF₃ | —CF₃ | 424 |
| 69 | —CH(Me)CH₂OMe | —CF₃ | —Cl | 407 |
| 70 | —CH(Me)CH₂OMe | —CF₃ | —CF₃ | 441 |
| 71 | —Me | —CF₂CF₂CF₃ | —F | 433 |
| 72 | —Me | —CF₂CF₂CF₃ | —Br | 495 |
| 130 | —CH₂CH₂SMe | —CF₃ | —Cl | 409 |
| 131 | —C(Me)₂CH₂C(Me)₃ | —CF₃ | —Cl | 447 |
| 132 | CH(Me)CH(Me)₂ | —CF₃ | —CF₃ | 439 |
| 133 | —C(Me)₂CF₃ | —CF₃ | —Br | 489 |
| 134 | —C(Me)₂CH2OMe | —CF₃ | —CF₃ | 455 |
| 135 | —C(Me)₂CF₃ | —CF₃ | —Me | 425 |
| 136 | —CH₂C(Me)₃ | —CF₃ | —Cl | 405 |
| 137 | —C(Me)₂CH₂OMe | —CF₃ | —Br | 465 |
| 138 | 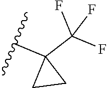 [SMILES: *C1(C(F)(F)F)CC1] | —CF₃ | —CF₃ | 475 |
| 139 | 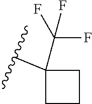 [SMILES: *C1(C(F)(F)F)CCC1] | —CF₃ | —CF₃ | 491 |
| 140 | —CH₂CH₂S(O)₂Me | —CF₃ | —CF₃ | 475 |
| 141 | —C(Me)₂CH₂C(Me)₃ | —CF₃ | —CF₃ | 481 |
| 142 | 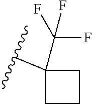 [SMILES: *C1(C(F)(F)F)CCC1] | —CF₃ | —Br | 502 |
| 143 | 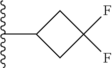 [SMILES: *C1CC(F)(F)C1] | —CF₃ | —Br | 470 |
| 144 | 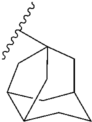 [SMILES: *C12CC3CCC(C1)C(C3)C2] | —CF₃ | —Br | 515 |
| 145 | 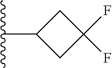 [SMILES: *C1CC(F)(F)C1] | —CF₃ | —CF₃ | 459 |
| 146 | —CH₂CH₂SMe | —CF₃ | —CF₃ | 443 |
| 147 | —C(Me)₂Ph | —CF₃ | —Cl | 453 |
| 148 | 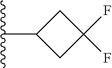 [SMILES: *C1CC(F)(F)C1] | —CF₃ | —Cl | 425 |
| 149 | 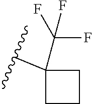 [SMILES: *C1(C(F)(F)F)CCC1] | —CF₃ | —Cl | 457 |
| 150 | —CH(Me)C(Me)₃ | —CF₃ | —CF₃ | 453 |
| 151 | —CH₂C(Me)₃ | —CF₃ | —CF₃ | 438 |
| 152 | 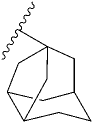 [SMILES: *C12CC3CCC(C1)C(C3)C2] | —CF₃ | —CF₃ | 503 |
| 153 | -cyclohexyl | —CF₃ | —Cl | 417 |
| 154 | —CH(Me)CH(Me)₂ | —CF₃ | —Cl | 405 |
| 155 | 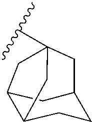 [SMILES: *C12CC3CCC(C1)C(C3)C2] | —CF₃ | —Cl | 469 |
| 156 | —CH(Me)C(Me)₃ | —CF₃ | —Cl | 419 |
| 157 | —C(Me)₂Ph | —CF₃ | —CF₃ | 487 |
| 158 | -cyclohexyl | —CF₃ | —CF₃ | 451 |
| 159 | —CH(Me)C(Me)₃ | —CF₃ | —Br | 464 |
| 179 | —C(Me)₂CF₃ | —CF₃ | —CF₃ | 479 |

| INDEX TABLE B | | | | |
|---|---|---|---|---|
| 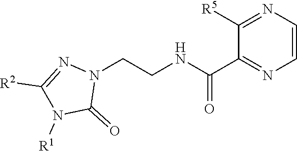 | | | | |
| Cmpd. No. | R¹ | R² | R⁵ | MS (M + 1) |
| 73 | —Me | —CF₃ | —Cl | 351 |

INDEX TABLE C

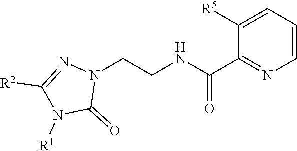

| Cmpd. | | | | |
|---|---|---|---|---|
| No. | R¹ | R² | R⁵ | MS (M + 1) |
| 74 | —Me | —CF₃ | —Cl | 350 |
| 75 | —Me | —CF₂CF₂CF₃ | —CF₃ | 483 |
| 76 | —CH₂CF₃ | —CF₃ | —CF₃ | 452 |
| 77 | —t-Bu | —CF₃ | —CF₃ | 426 |
| 78 | —CH₂c-Pr | —CF₃ | —CF₃ | 424 |
| 79 | —i-Pr | —CF₃ | —CF₃ | 412 |
| 80 | —CH₂(c-Bu) | —CF₃ | —Cl | 404 |
| 81 | —CH₂(c-Bu) | —CF₃ | —CF₃ | 438 |
| 82 | —-Pr(Et) | —CF₃ | —Cl | 406 |
| 83 | —-Pr(Et) | —CF₃ | —CF₃ | 440 |
| 84 | —CH(Me)c-Pr | —CF₃ | —CF₃ | 438 |
| 85 | —i-Bu | —CF₃ | —CF₃ | 426 |
| 86 | —c-Pr | —CF₃ | —CF₃ | 410 |
| 87 | —Me | —CF₂CF₃ | —Cl | 400 |
| 88 | —Me | —CF₂CF₂CF₃ | —Cl | 451 |
| 89 | —Me | —CF₂CF₃ | —CF₃ | 434 |
| 90 | —Me | —CF₃ | —CF₃ | 384 |
| 160 | —C(Me)₂CF₃ | —CF₃ | —CF₃ | 480 |
| 161 | —CH(Me)CH(Me)₂ | —CF₃ | —CF₃ | 440 |
| 162 | —CH(Me)C(Me)₃ | —CF₃ | —CF₃ | 454 |
| 163 | 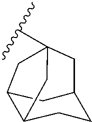 [SMILES: *C12CC3CCC(C1)C(C3)C2] | —CF₃ | —CF₃ | 504 |
| 164 | —CH(Me)CF₃ | —CF₃ | —CF₃ | 466 |

INDEX TABLE D

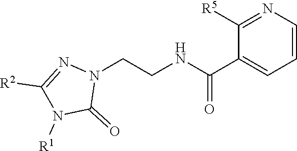

| Cmpd. | | | | |
|---|---|---|---|---|
| No. | R¹ | R² | R⁵ | MS (M + 1) |
| 91 | —CH₂(i-Bu) | —CF₃ | —CF₃ | 440 |
| 92 | —t-Bu | —CF₃ | —Cl | 392 |
| 93 | —t-Bu | —CF₃ | —CF₃ | 426 |
| 94 | —Me | —CF₂CF₃ | —CF₃ | 434 |
| 95 | —c-Pr | —CF₃ | —CF₃ | 410 |
| 96 | —c-Pr | —CF₃ | —Cl | 376 |
| 97 | 1-methylcyclopropyl | —CF₃ | —CF₃ | 424 |
| 98 | —CH₂(c-Pr) | —CF₃ | —CF₃ | 424 |
| 99 | —CH₂CF₃ | —CF₃ | —CF₃ | 451 |
| 100 | —i-Pr | —CF₃ | —CF₃ | 412 |
| 101 | —CH₂(c-Bu) | —CF₃ | —Cl | 404 |
| 102 | —CH(Me)c-Pr | —CF₃ | —CF₃ | 438 |
| 103 | —CH₂(i-Bu) | —CF₃ | —Cl | 406 |
| 104 | —Me | —CF₃ | —CF₃ | 384 |
| 116 | —-Pr(Et) | —CF₃ | —CF₃ | 440 |
| 120 | —CH₂C(O)Me | —CF₃ | —CF₃ | 424 |
| 121 | —CH(Me)CH₂OMe | —CF₃ | —CF₃ | 442 |
| 122 | —CH₂CH₂(c-Pr) | —CF₃ | —Cl | 404 |
| 126 | —CH₂CHF₂ | —CF₃ | —CF₃ | 434 |
| 165 | —C(Me)₂CF₃ | —CF₃ | —CF₃ | 480 |
| 166 | —CH(Me)CF₃ | —CF₃ | —Cl | 432 |
| 167 | —CH(Me)CH(Me)₂ | —CF₃ | —CF₃ | 440 |
| 168 | —C(Me)₂CH₂OMe | —CF₃ | —CF₃ | 456 |
| 169 | 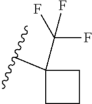 [SMILES: *C1(C(F)(F)F)CCC1] | —CF₃ | —CF₃ | 492 |
| 170 | 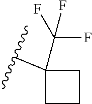 [SMILES: *C1(C(F)(F)F)CCC1] | —CF₃ | —Cl | 458 |
| 171 | —C(Me)₂CF₃ | —CF₃ | —Cl | 446 |
| 172 | —CH(Me)C(Me)₃ | —CF₃ | —CF₃ | 454 |
| 173 | 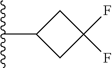 [SMILES: *C1CC(F)(F)C1] | —CF₃ | —CF₃ | 460 |
| 174 | —CH(Me)C(Me)₃ | —CF₃ | —Cl | 420 |
| 175 | —CH(Me)CF₃ | —CF₃ | —CF₃ | 466 |

| INDEX TABLE E | | | | | |
|---|---|---|---|---|---|
| 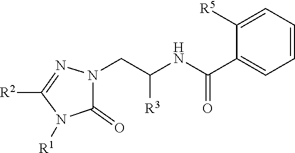 | | | | | |
| Cmpd. No. | R¹ | R² | R³ | R⁵ | MS (M + 1) |
| 105 | —Me | —CF₃ | —Me | —F | 347 |
| 106 | —Me | —CF₃ | —Me | —Br | 409 |
| 129 | —Me | —CF₃ | —Me | —Cl | 362 |

| INDEX TABLE F | | | | | |
|---|---|---|---|---|---|
| 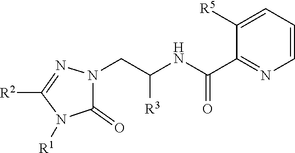 | | | | | |
| Cmpd. No. | R¹ | R² | R³ | R⁵ | MS (M + 1) |
| 107 | —Me | —CF₃ | —Me | —CF₃ | 398 |
| 108 | —Me | —CF₃ | —Me | —Cl | 364 |
| 126 | —t-Bu | —CF₃ | —Me | —CF₃ | 439 |

[symbol]

[symbol]

indicates data missing or illegible when filed

INDEX TABLE G

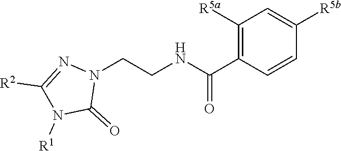

| Cmpd. No. | R¹ | R² | R^{5a} | R^{5b} | MS (M + 1) |
|---|---|---|---|---|---|
| 109 | —t-Bu | —CF₃ | —CF₃ | —Cl | 459 |
| 110 | —t-Bu | —CF₃ | —CF₃ | —CF₃ | 493 |
| 111 | —t-Bu | —CF₃ | —CF₃ | —Me | 439 |
| 112 | —CH₂C(O)Me | —CF₃ | —CF₃ | —F | 441 |
| 113 | —CH₂(Me)CH₂OMe | —CF₃ | —CF₃ | —F | 459 |
| 114 | —t-Bu | —CF₃ | —CF₃ | —Br | 504 |
| 115 | —t-Bu | —CF₃ | —CF₃ | —F | 443 |

INDEX TABLE H

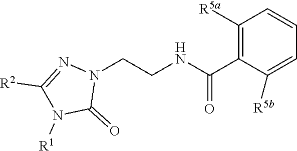

| Cmpd. No. | R¹ | R² | R^{5a} | R^{5b} | MS (M + 1) |
|---|---|---|---|---|---|
| 117 | —CH(Me)CH₂OMe | —CF₃ | —CF₃ | —F | 459 |
| 118 | —t-Bu | —CF₃ | —CF₃ | —Cl | 459 |
| 119 | —CH₂C(O)Me | —CF₃ | —CF₃ | —F | 441 |
| 128 | —t-Bu | —CF₃ | —CF₃ | —F | 443 |

INDEX TABLE I

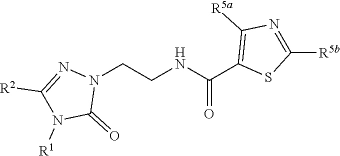

| Cmpd. No. | R¹ | R² | R^{5a} | R^{5b} | MS (M + 1) |
|---|---|---|---|---|---|
| 123 | —CH₂(cyclohexyl) | —CF₃ | —CF₃ | —Me | 486 |
| 176 | —C(Me)₂CF₃ | —CF₃ | —CF₃ | —Me | 500 |

INDEX TABLE J

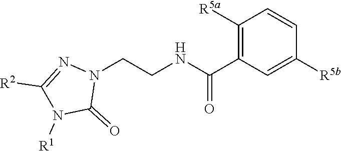

| Cmpd. No. | R¹ | R² | R^{5a} | R^{5b} | MS (M + 1) |
|---|---|---|---|---|---|
| 124 | —t-Bu | —CF₃ | —CF₃ | —Cl | 459 |

| INDEX TABLE K | | | | | |
|---|---|---|---|---|---|
| 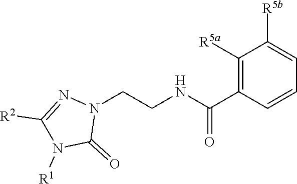 | | | | | |
| Cmpd. No. | R¹ | R² | R^{5a} | R^{5b} | MS (M + 1) |
| 125 | —t-Bu | —CF₃ | —CF₃ | —F | 443 |

| INDEX TABLE L | | | | | |
|---|---|---|---|---|---|
| 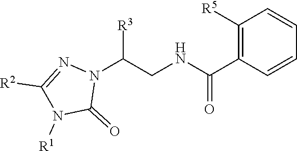 | | | | | |
| Cmpd. No. | R¹ | R² | R³ | R⁵ | MS (M + 1) |
| 127 | —t-Bu | —CF₃ | —Me | —CF₃ | 439 |

INDEX TABLE M

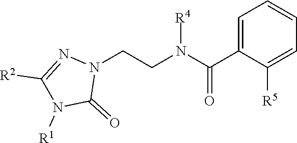

| Cmpd. No. | R¹ | R² | R⁴ | R⁵ | MS (M + 1) |
|---|---|---|---|---|---|
| 177 | —C(Me)₃ | —CF₃ | —C(O)OMe | —CF₃ | 483 |
| 178 | —C(Me)₃ | —CF₃ | —Me | —CF₃ | 439 |

The following Tests demonstrate the control efficacy of compounds of this disclosure on specific pests. “Control efficacy” represents inhibition of invertebrate pest development (including mortality) that causes significantly reduced feeding. The pest control protection afforded by the compounds is not limited, however, to these species. See Index Tables A-M for compound descriptions.

### Biological Examples

### Nematicide Assay to Evaluate Compounds Potency

The following Test demonstrates the potency of compounds of this disclosure on controlling specific invertebrates. “Potency” represents inhibition of invertebrate development that causes significantly reduced feeding on the plant roots and reduction of galling (development structures of the invertebrate which represent the invertebrate's survival and establishment on the root). The invertebrate control protection afforded by the compounds is not limited, however, to the Root Knot Nematode species, only.

### Formulation and Application Methodology

Test compounds were formulated using a solution containing 25% acetone and 75% water. The formulated compounds were applied in 330 μl of liquid through a drip applicator that was positioned above the top of each test unit. Test compounds were applied at 500 ppm and 100 ppm and each test was replicated three times.

For evaluating control of Root Knot Nematodes the test unit consisted of a small open container with a 4-day-old host plant inside. The formulated compounds were dripped into the container and the nematodes were applied following the compound's application. The test units were held for 8 days in a growth chamber at 29.4° C. or 85 F. Root invasion by the nematodes was then visually assessed based on presence or absence of galls on the roots. Compounds of Formula 1 as disclosed herein have eliminated the presence of Root Knot Nematode galls from the roots at 500 ppm and 100 ppm.

Of the compounds tested at 500 ppm, the following provided excellent control efficacy (100% efficacy, i.e. the roots have no nematode galls compared to the inoculated control): Compound 2, Compound 1, Compound 14, Compound 12, Compound 11, Compound 9, Compound 10, Compound 8, Compound 24, Compound 35, Compound 33, Compound 32, Compound 31, Compound 30, Compound 29, Compound 28, Compound 27, Compound 41, Compound 38, Compound 128, Compound 45, Compound 93, Compound 50, Compound 92, Compound 64, Compound 63, Compound 59, Compound 56, and Compound 55, Compound 165, Compound 160, Compound 166, Compound 133, Compound 135, Compound 138, Compound 139, Compound 142, Compound 169, Compound 143, Compound 145, Compound 148, Compound 170, Compound 149, Compound 171, Compound 173, Compound 175, and Compound 179.

Of the compounds tested at 500 ppm, the following provided excellent control efficacy (50%-<100% efficacy, i.e. the roots have 50%-<100% decreased nematode gall formation compared to the inoculated control): Compound 90, Compound 104, Compound 7, Compound 6, Compound 129, Compound 88, Compound 4, Compound 3, Compound 106, Compound 15, Compound 5, Compound 34, Compound 102, Compound 115, Compound 100, Compound 99, Compound 97, Compound 44, Compound 96, Compound 95, Compound 125, Compound 94, Compound 77, Compound 51, Compound 49, Compound 75, Compound 46, Compound 61, Compound 54, Compound 72, and Compound 122, Compound 134, Compound 168, Compound 152, Compound 176, Compound 155, and Compound 164.

Of the compounds tested at 100 ppm, the following provided excellent control efficacy (100% efficacy, i.e. that roots have not nematode galls compared to the inoculated control): Compound 24, Compound 92, and Compound 64, Compound 165, Compound 133, Compound 138, Compound 145, Compound 148, Compound 171, and Compound 179.

Of the compounds tested at 100 ppm, the following provided excellent control efficacy (50%-<100% efficacy, i.e. that roots have 50%-<100% decreased nematode gall formation compared to the inoculated control): Compound 2, Compound 1, Compound 14, Compound 12, Compound 11, Compound 9, Compound 10, Compound 8, Compound 6, Compound 3, Compound 33, Compound 32, Compound 31, Compound 30, Compound 27, Compound 41, Compound 38, Compound 100, Compound 44, Compound 50, Compound 63, Compound 59, and Compound 56, Compound 160, Compound 166, Compound 135, Compound 139, Compound 168, Compound 142, Compound 169, Compound 143, Compound 170, Compound 149, Compound 173, and Compound 152.

What is claimed is:

1. A compound selected from Formula 1, N-oxides and salts thereof, 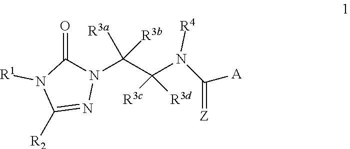
wherein
A is phenyl, pyridyl or pyrazinyl, each substituted with 1-3 R⁵, provided that at least one R⁵ is in the ortho position;
R¹ is C₁-C₆ alkyl, C₃-C₆ alkenyl, C₃-C₆ alkynyl, C₃-C₈ cycloalkyl or C₁-C₆ alkoxy, each R¹ optionally substituted with halogen, C₁-C₄ alkyl, C₁-C₄ haloalkyl, C₃-C₆ cycloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;
R₂ is C₁-C₃ haloalkyl or halogen;
Z is O or S
R^{3a} and R^{3b} are each independently H, C₁-C₂ alkyl, C₁-C₂ haloalkyl, or R^{3a} and R^{3b} are taken together to form a 3- to 5-membered carbocyclic ring;
R^{3c} and R^{3d} are each independently H, C₁-C₂ alkyl, C₁-C₂ haloalkyl, or R^{3c} and R^{3d} are taken together to form a 3- to 5-membered carbocyclic ring;
R⁴ is H, C₁-C₄ alkyl, C₂-C₄ alkylcarbonyl or C₁-C₄ alkoxycarbonyl;
R⁵ is halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄haloalkylsulfonyl;
with the proviso that when R¹ is Me, R² is not CF3.

2. A composition comprising a compound of Formula 1′, N-oxides, and salts thereof, the compound of Formula 1′ having the structure: 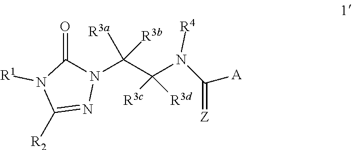
wherein
A is phenyl, pyridyl or pyrazinyl, each substituted with 1-3 R⁵, provided that at least one R⁵ is in the ortho position;
R¹ is C₁-C₆ alkyl, C₃-C₆ alkenyl, C₃-C₆ alkynyl, C₃-C₈ cycloalkyl or C₁-C₆ alkoxy, each R¹ optionally substituted with halogen, C₁-C₄ alkyl, C₁-C₄ haloalkyl, C₃-C₆ cycloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl;
R₂ is C₁-C₃ haloalkyl or halogen;
Z is O or S
R^{3a} and R^{3b} are each independently H, C₁-C₂ alkyl, C₁-C₂ haloalkyl, or R^{3a} and R^{3b} are taken together to form a 3- to 5-membered carbocyclic ring;
R^{3c} and R^{3d} are each independently H, C₁-C₂ alkyl, C₁-C₂ haloalkyl, or R^{3c} and R^{3d} are taken together to form a 3- to 5-membered carbocyclic ring;
R⁴ is H, C₁-C₄ alkyl, C₂-C₄ alkylcarbonyl or C₁-C₄ alkoxycarbonyl;
each R⁵ is independently halogen, C₁-C₄ alkyl, C₁-C₄ haloalkyl, cyano, nitro, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalksulfinyl, C₁-C₄ alkylsulfonyl or C₁-C₄ haloalkylsulfonyl.

3. The compound of claim 1 wherein:
A is

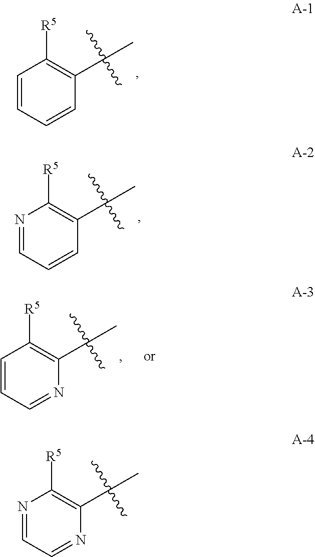

4. The compound of claim 1 or claim 3 wherein A is A-1 or A-2.

5. The compound of claim 1, or any one of claim 3-4 wherein A is A-1.

6. The compound of any one of claim 1, or any one of claims 3 to 5 wherein R¹ is C₁-C₄ alkyl, C₂-C₄ haloalkyl, C₃-C₄ cycloalkyl, or CH₂(C₃-C₄ cycloalkyl).

7. The compound of any one of claim 1, or 3 to 6 wherein R¹ is Me, C(Me)₂CF₃, or t-Bu.

8. The compound of any one of claim 1, or 3 to 7 wherein R1 is t-Bu or Me.

9. The compound of any one of claim 1, or 3 to 8 wherein R² is C(Cl)F₂, CF₃, CF₂CF₃, CHF₂, or CF₂CF₂CF₃.

10. The composition of claim 2, the composition comprising the compound of Formula 1′
wherein A is

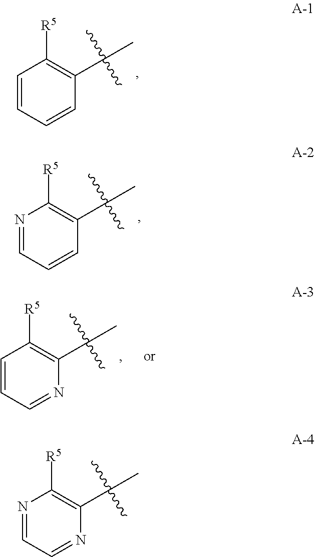

11. The composition of claim 2 wherein A is A-1.

12. The composition of claim 2 or any one of claims 13 to 14 wherein R¹ is C₁-C₄ alkyl, C₂-C₄ haloalkyl, C₃-C₄ cycloalkyl, or CH₂(C₃-C₄ cycloalkyl).

13. The composition of claim 2 or any one of claims 13 to 15 wherein R¹ is Me.

14. The compound of claim 1 wherein the compound is selected from:
N-[2-[4-(1,1-Dimethylethyl)-4,5-dihydro-5-oxo-3-(trifluoromethyl)-1H-1,2,4-triazol-1-yl]ethyl]-2-(trifluoromethyl)benzamide,
N-[2-[4-(Cyclopropylmethyl)-4,5-dihydro-5-oxo-3-(trifluoromethyl)-1H-1,2,4-triazol-1-yl]ethyl]-2-(trifluoromethyl)benzamide,
N-[2-[4-(Cyclopropyl-4,5-dihydro-5-oxo-3-(trifluoromethyl)-1H-1,2,4-triazol-1-yl]ethyl]-2-(trifluoromethyl)benzamide,
2-Chloro-N-[2-[4-(1,1-dimethylethyl)-4,5-dihydro-5-oxo-3-(trifluoromethyl)-1H-1,2,4-triazol-1-yl]ethyl]benzamide,
2-Chloro-N-[2-[4-(1,1-dimethylethyl)-4,5-dihydro-5-oxo-3-(trifluoromethyl)-1H-1,2,4-triazol-1-yl]ethyl]-3-pyridinecarboxamide,
2-Bromo-N-[2-[4-(1,1-dimethylethyl)-4,5-dihydro-5-oxo-3-(trifluoromethyl)-1H-1,2,4-triazol-1-yl]ethyl]benzamide, and N-[2-[4,5-Dihydro-5-oxo-4-(2,2,2-trifluoro-1,1-dimethylethyl)-3-(trifluoromethyl)-1H-1,2,4-triazol-1-yl]ethyl]-2-(trifluoromethyl)benzamide.

15. The composition of claim 2 wherein the compound of Formula 1′ is selected from:
N-[2-[4-(1,1-Dimethylethyl)-4,5-dihydro-5-oxo-3-(trifluoromethyl)-1H-1,2,4-triazol-1-yl]ethyl]-2-(trifluoromethyl)benzamide
N-[2-[4,5-Dihydro-4-methyl-5-oxo-3-(trifluoromethyl)-1H-1,2,4-triazol-1-yl]ethyl]-2-(trifluoromethyl)benzamide
N-[2-[4-(Cyclopropylmethyl)-4,5-dihydro-5-oxo-3-(trifluoromethyl)-1H-1,2,4-triazol-1-yl]ethyl]-2-(trifluoromethyl)benzamide
N-[2-[4-(Cyclopropyl-4,5-dihydro-5-oxo-3-(trifluoromethyl)-1H-1,2,4-triazol-1-yl]ethyl]-2-(trifluoromethyl)benzamide
2-Chloro-N-[2-[4-(1,1-dimethylethyl)-4,5-dihydro-5-oxo-3-(trifluoromethyl)-1H-1,2,4-triazol-1-yl]ethyl]benzamide
2-Chloro-N-[2-[4-(1,1-dimethylethyl)-4,5-dihydro-5-oxo-3-(trifluoromethyl)-1H-1,2,4-triazol-1-yl]ethyl]-3-pyridinecarboxamide,
2-Bromo-N-[2-[4-(1,1-dimethylethyl)-4,5-dihydro-5-oxo-3-(trifluoromethyl)-1H-1,2,4-triazol-1-yl]ethyl]benzamide, and
N-[2-[4,5-Dihydro-5-oxo-4-(2,2,2-trifluoro-1,1-dimethylethyl)-3-(trifluoromethyl)-1H-1,2,4-triazol-1-yl]ethyl]-2-(trifluoromethyl)benzamide.

16. A composition comprising a compound of any one of claim 1 or 2 and at least one additional component selected from surfactants, solid diluents and liquid diluents, said composition optionally further comprising at least one additional biologically active compound or agent.

17. The composition of claim 16 wherein the at least one additional biologically active compound or agent is selected from abamectin, acephate, acequinocyl, acetamiprid, acrinathrin, afidopyropen, amidoflumet, amitraz, avermectin, azadirachtin, azinphos-methyl, benfuracarb, bensultap, bifenthrin, bifenazate, bistrifluron, borate, buprofezin, carbaryl, carbofuran, cartap, carzol, chlorantraniliprole, chlorfenapyr, chlorfluazuron, chlorpyrifos, chlorpyrifos-methyl, chromafenozide, clofentezin, clothianidin, cyantraniliprole, cyclaniliprole, cyclobutrifluram, cycloprothrin, cycloxaprid, cyetpyrafen, cyflumetofen, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, diafenthiuron, diazinon, dieldrin, diflubenzuron, dimefluthrin, dimehypo, dimethoate, dinotefuran, diofenolan, emamectin, endosulfan, esfenvalerate, ethiprole, etofenprox, etoxazole, fenbutatin oxide, fenitrothion, fenothiocarb, fenoxycarb, fenpropathrin, fenvalerate, fipronil, flometoquin, flonicamid, flubendiamide, flucythrinate, flufenerim, flufenoxuron, flufenoxystrobin, fluensulfone, fluopyram, flupentiofenox, flupyradifurone, fluvalinate, tau-fluvalinate, fonophos, formetanate, fosthiazate, halofenozide, heptafluthrin, hexaflumuron, hexythiazox, hydramethylnon, imidacloprid, indoxacarb, insecticidal soaps, isofenphos, lufenuron, malathion, meperfluthrin, metaflumizone, metaldehyde, methamidophos, methidathion, methiocarb, methomyl, methoprene, methoxychlor, methoxyfenozide, metofluthrin, monocrotophos, monofluorothrin, nicofluprole, nicotine, nitenpyram, nithiazine, novaluron, noviflumuron, N-[1,1-dimethyl-2-(methylthio)ethyl]-7-fluoro-2-(3-pyridinyl)-2H-indazole-4-carboxamide, N-[1,1-dimethyl-2-(methylsulfinyl)ethyl]-7-fluoro-2-(3-pyridinyl)-2H-indazole-4-carboxamide, N-[1,1-dimethyl-2-(methylsulfonyl)ethyl]-7-fluoro-2-(3-pyridinyl)-2H-indazole-4-carboxamide, N-(1-methylcyclopropyl)-2-(3-pyridinyl)-2H-indazole-4-carboxamide and N-[1-(difluoromethyl)cyclopropyl]-2-(3-pyridinyl)-2H-indazole-4-carboxamide, oxamyl, parathion, parathion-methyl, permethrin, phorate, phosalone, phosmet, phosphamidon, pirimicarb, profenofos, profluthrin, propargite, protrifenbute, pyflubumide, pymetrozine, pyrafluprole, pyrethrin, pyridaben, pyridalyl, pyrifluquinazon, pyriminostrobin, pyriprole, pyriproxyfen, rotenone, ryanodine, silafluofen, spinetoram, spinosad, spirodiclofen, spiromesifen, spirotetramat, sulprofos, sulfoxaflor, tebufenozide, tebufenpyrad, teflubenzuron, tefluthrin, tetrachlorvinphos, tetramethrin, tetramethylfluthrin, thiacloprid, thiamethoxam, thiodicarb, thiosultap-sodium, tioxazafen, tolfenpyrad, tralomethrin, triazamate, trichlorfon, triflumezopyrim, triflumuron, Bacillus thuringiensis delta-endotoxins, entomopathogenic bacteria, entomopathogenic viruses and entomopathogenic fungi.

18. A composition for protecting an organism from an invertebrate parasitic pest comprising a parasiticidally effective amount of a compound of any one of claim 1 or 2.

19. A method for controlling an invertebrate parasitic pest comprising contacting an organism in need thereof and/or the organism's environment with a biologically effective amount of a compound of any one of claim 1 or 2.

20. A treated seed comprising a compound of any one of claim 1 or 2 amount of from about 0.0001 to 1% by weight of the seed before treatment.
